SEQUENCE LISTING (91 sequences)

SEQ ID NO: 1 - protein, 12 aa, synthetic construct
GRKKRRQRRRPQ

SEQ ID NO: 2 - protein, 18 aa, synthetic construct
KLALKLALKALKAALKLA

SEQ ID NO: 3 - protein, 15 aa, synthetic construct
RQIKWFQNRRMKWKK

SEQ ID NO: 4 - protein, 16 aa, synthetic construct
RQIRIWFQNRRMRWRR

SEQ ID NO: 5 - protein, 17 aa, synthetic construct
CSIPPEVKFNKPFVYLI

SEQ ID NO: 6 - protein, 13 aa, synthetic construct
FVQWFSKFLGRIL

SEQ ID NO: 7 - protein, 7 aa, synthetic construct
LPHLKLC

SEQ ID NO: 8 - protein, 18 aa, synthetic construct
LLIILRRRIRKQAHAHSK

SEQ ID NO: 9 - protein, 24 aa, synthetic construct
LGTYTQDFNKFHTFPQTAIGVGAP

SEQ ID NO: 10 - protein, 30 aa, synthetic construct
GAAEAAARVYDLGLRRLRQRRRLRRERVRA

SEQ ID NO: 11 - protein, 18 aa, synthetic construct
RQIKIWFQNRRMKWKKGG

SEQ ID NO: 12 - protein, 30 aa, synthetic construct
RRRQRRKKRGGDIMGEWGNEIFGAIAGFLG

SEQ ID NO: 13 - protein, 10 aa, synthetic construct
GRKKRRQRRR

SEQ ID NO: 14 - protein, 18 aa, synthetic construct
RGGRLSYSRRRFSTSTGR

SEQ ID NO: 15 - protein, 10 aa, synthetic construct
RRLSYSRRRF

SEQ ID NO: 16 - protein, 11 aa, synthetic construct
PIRRRKKLRRL

SEQ ID NO: 17 - protein, 12 aa, synthetic construct
RRQRRTSKLMKR

SEQ ID NO: 18 - protein, 15 aa, synthetic construct
RRRRNRTRRNRRRVR

SEQ ID NO: 19 - protein, 19 aa, synthetic construct
KMTRAQRRAAARRNRWTAR

SEQ ID NO: 20 - protein, 13 aa, synthetic construct
TRRQRTRRARRNR

SEQ ID NO: 21 - protein, 13 aa, synthetic construct
GRKKRRQRRRPPQ

SEQ ID NO: 22 - protein, 13 aa, synthetic construct
GRRRRRRRRRPPQ

SEQ ID NO: 23 - protein, 27 aa, synthetic construct
GWTLNSAGYLLGKINLKALAALAKKIL

SEQ ID NO: 24 - protein, 27 aa, synthetic construct
MGLGLHLLVLAAALQGAWSQPKKKRKV

SEQ ID NO: 25 - protein, 27 aa, synthetic construct
GALFLGWLGAAGSTMGAWSQPKKKRKV

SEQ ID NO: 26 - protein, 27 aa, synthetic construct
GALFLGFLGAAGSTMGAWSQPKKKRKV

SEQ ID NO: 27 - protein, 27 aa, synthetic construct
GALFLGFLGAAGSTMGAWSQPKSKRKV

SEQ ID NO: 28 - protein, 21 aa, synthetic construct
KETWWETWWTEWSQPKKKRKV

SEQ ID NO: 29 - protein, 21 aa, synthetic construct
KETWFETWFTEWSQPKKKRKV

SEQ ID NO: 30 - protein, 29 aa, synthetic construct
YTIWMPENPRPGTPCDIFTNSRGKRASNG

SEQ ID NO: 31 - RNA, 100 nt, synthetic construct
  source 1..100: mol_type = other RNA
nnnnnnnnnnnnnnnnnnnngttttagagctagaaatagcaagttaaaataaggctagtccgttatcaacttgaaaaagtggcaccgagtcggtgctttt

SEQ ID NO: 32 - RNA, 180 nt, synthetic construct
  source 1..180: mol_type = other RNA
nnnnnnnnnnnnnnnnnnnngtttaagagctatgctgcgaatacgagatgcggccgccgaccagaatcatgcaagtgcgtaagatagtcgcgggtcggcggctcgtattcgcagcatagcaagtttaaataaggctagtccgttatcaacttgaaaaagtggcaccgagtcggtgctttt

SEQ ID NO: 33 - RNA, 183 nt, synthetic construct
  source 1..183: mol_type = other RNA
nnnnnnnnnnnnnnnnnnnngtttaagagctatgctggaaacagcatagcaagtttaaaaaggctagtccgttatcaacttcgaatacgagatgcggccgccgaccagaatcatgcaagtgcgtaagatagtcgcgggtcggcggctcgtattcggaaaaagtggcaccgagtcggtgctttt

SEQ ID NO: 34 - RNA, 184 nt, synthetic construct
  source 1..184: mol_type = other RNA
nnnnnnnnnnnnnnnnnnnngtttaagagctatgctggaaacagcatagcaagtttaaataaggctagtccgttatcaacttgaaaaagtggcaccgagtcggtgccgaatacgagatgcggccgccgaccagaatcatgcaagtgcgtaagatagtcgcgggtcggcggctcgtattcgtttt

SEQ ID NO: 35 - RNA, 42 nt, synthetic construct
  source 1..42: mol_type = other RNA
nnnnnnnnnnnnnnnnnnnngttttagagctatgctgttttg

SEQ ID NO: 36 - RNA, 64 nt, synthetic construct
  source 1..64: mol_type = other RNA
tagcaagttaaaataaggctagtccgttatcaacttgaaaaagtggcaccgagtcggtgctttt

SEQ ID NO: 37 - RNA, 82 nt, synthetic construct
  source 1..82: mol_type = other RNA
ggaaccattcaaaacagcatagcaagttaaaataaggctagtccgttatcaacttgaaaaagtgcaccgagtcggtgctttt

SEQ ID NO: 38 - RNA, 41 nt, synthetic construct
  source 1..41: mol_type = other RNA
taatttctactcttgtagatnnnnnnnnnnnnnnnnnnnnn

SEQ ID NO: 39 - RNA, 46 nt, synthetic construct
  source 1..46: mol_type = other RNA
ggtaatttctactaagtgtagatnnnnnnnnnnnnnnnnnnnnnnn

SEQ ID NO: 40 - DNA, 56 bp, synthetic construct
  source 1..56: mol_type = other DNA
gtcggatcactgagcgagcgatctgagaagtggcacnnnnnnnnnnnnnnnnnnnn

SEQ ID NO: 41 - DNA, 96 bp, synthetic construct
  source 1..96: mol_type = other DNA
gtctaaaggacagaattttcaacgggtgtgccaatggccactttccaggtggcaaagcccgttgaacttctcaaaaagaacgctcagtgttctgac

SEQ ID NO: 42 - RNA, 64 nt, synthetic construct
  source 1..64: mol_type = other RNA
gatttagactaccccaaaaacgaaggggactaaaacnnnnnnnnnnnnnnnnnnnnnnnnnnnn

SEQ ID NO: 43 - RNA, 66 nt, synthetic construct
  source 1..66: mol_type = other RNA
nnnnnnnnnnnnnnnnnnnnnnnnnnnnnngttgtggaaggtccagttttgggggctattacaaca

SEQ ID NO: 44 - RNA, 53 nt, synthetic construct
  source 1..53: mol_type = other RNA
aacccctaccaactggtcggggtttgaaacnnnnnnnnnnnnnnnnnnnnnnn

SEQ ID NO: 45 - protein, 12 aa, synthetic construct
CYGRKKRRQRRR

SEQ ID NO: 46 - DNA, 19 bp, synthetic construct
  source 1..19: mol_type = other DNA
ccaccaactgggaggaaag

SEQ ID NO: 47 - DNA, 23 bp, synthetic construct
  source 1..23: mol_type = other DNA
ccacagtcttctgggtggcagtg

SEQ ID NO: 48 - DNA, 22 bp, synthetic construct
  source 1..22: mol_type = other DNA
ggaatcctgcacccacttcttc

SEQ ID NO: 49 - DNA, 22 bp, synthetic construct
  source 1..22: mol_type = other DNA
tttccgcttcctgaggctggat

SEQ ID NO: 50 - DNA, 22 bp, synthetic construct
  source 1..22: mol_type = other DNA
gacgtttcagaggttctcagag

SEQ ID NO: 51 - DNA, 22 bp, synthetic construct
  source 1..22: mol_type = other DNA
gttcatctcggagcctgtagtg

SEQ ID NO: 52 - DNA, 23 bp, synthetic construct
  source 1..23: mol_type = other DNA
agtggtatagacaggtctgttgg

SEQ ID NO: 53 - DNA, 23 bp, synthetic construct
  source 1..23: mol_type = other DNA
ccgatggaaaacaggcaagagtc

SEQ ID NO: 54 - DNA, 23 bp, synthetic construct
  source 1..23: mol_type = other DNA
cggttagcagtatgttgtccagc

SEQ ID NO: 55 - DNA, 23 bp, synthetic construct
  source 1..23: mol_type = other DNA
cctcatagatgctaccaaggcac

SEQ ID NO: 56 - DNA, 23 bp, synthetic construct
  source 1..23: mol_type = other DNA
ccacctgtgagttcttcaaaggc

SEQ ID NO: 57 - DNA, 22 bp, synthetic construct
  source 1..22: mol_type = other DNA
tgggtcctgtagatggcattgc

SEQ ID NO: 58 - DNA, 21 bp, synthetic construct
  source 1..21: mol_type = other DNA
ttcatgtggtggtccagcttt

SEQ ID NO: 59 - DNA, 22 bp, synthetic construct
  source 1..22: mol_type = other DNA
aaccaagggagcttcagggtca

SEQ ID NO: 60 - DNA, 22 bp, synthetic construct
  source 1..22: mol_type = other DNA
gtctggacccattccttcttgg

SEQ ID NO: 61 - DNA, 22 bp, synthetic construct
  source 1..22: mol_type = other DNA
atgacacctggctgggagcaaa

SEQ ID NO: 62 - DNA, 22 bp, synthetic construct
  source 1..22: mol_type = other DNA
ctgtctgcctcttttggtcagg

SEQ ID NO: 63 - DNA, 22 bp, synthetic construct
  source 1..22: mol_type = other DNA
acacacttggcggttccttcga

SEQ ID NO: 64 - DNA, 23 bp, synthetic construct
  source 1..23: mol_type = other DNA
gccatagaactgatgagagggag

SEQ ID NO: 65 - DNA, 21 bp, synthetic construct
  source 1..21: mol_type = other DNA
tccaggtcatagagaggctca

SEQ ID NO: 66 - DNA, 25 bp, synthetic construct
  source 1..25: mol_type = other DNA
gcgtgttagtgtaaatgaacttcta

SEQ ID NO: 67 - DNA, 23 bp, synthetic construct
  source 1..23: mol_type = other DNA
tgaggccggtgctgagtatgtcg

SEQ ID NO: 68 - DNA, 22 bp, synthetic construct
  source 1..22: mol_type = other DNA
gctacaggagaatcaccagtgg

SEQ ID NO: 69 - DNA, 23 bp, synthetic construct
  source 1..23: mol_type = other DNA
cagcaacagcaaggcgaaaaagg

SEQ ID NO: 70 - DNA, 22 bp, synthetic construct
  source 1..22: mol_type = other DNA
cgaagactacagttctgccatt

SEQ ID NO: 71 - DNA, 22 bp, synthetic construct
  source 1..22: mol_type = other DNA
tggaccttccaggatgaggaca

SEQ ID NO: 72 - DNA, 21 bp, synthetic construct
  source 1..21: mol_type = other DNA
ctgcaagagacttccatccag

SEQ ID NO: 73 - DNA, 22 bp, synthetic construct
  source 1..22: mol_type = other DNA
tccaccgtcaaaatgcagctgc

SEQ ID NO: 74 - DNA, 22 bp, synthetic construct
  source 1..22: mol_type = other DNA
cgggaagacaataactgcaccc

SEQ ID NO: 75 - DNA, 22 bp, synthetic construct
  source 1..22: mol_type = other DNA
acgagagttgcctggctactag

SEQ ID NO: 76 - DNA, 22 bp, synthetic construct
  source 1..22: mol_type = other DNA
ttgaactggcgttggaagcacg

SEQ ID NO: 77 - DNA, 22 bp, synthetic construct
  source 1..22: mol_type = other DNA
aacggcagcatggtatggagtg

SEQ ID NO: 78 - DNA, 17 bp, synthetic construct
  source 1..17: mol_type = other DNA
gaagctcagtgccgcca

SEQ ID NO: 79 - DNA, 22 bp, synthetic construct
  source 1..22: mol_type = other DNA
tccagagcttgaaggtgttgcc

SEQ ID NO: 80 - DNA, 22 bp, synthetic construct
  source 1..22: mol_type = other DNA
gctacaagaggatcaccagcag

SEQ ID NO: 81 - DNA, 22 bp, synthetic construct
  source 1..22: mol_type = other DNA
actgcctgctgcttctcctaca

SEQ ID NO: 82 - DNA, 22 bp, synthetic construct
  source 1..22: mol_type = other DNA
accctcccacttcctgctgttt

SEQ ID NO: 83 - DNA, 22 bp, synthetic construct
  source 1..22: mol_type = other DNA
cctgctgctttgcctacctctc

SEQ ID NO: 84 - DNA, 22 bp, synthetic construct
  source 1..22: mol_type = other DNA
ggtgcctatgtctcagcctctt

SEQ ID NO: 85 - DNA, 20 bp, synthetic construct
  source 1..20: mol_type = other DNA
ctggcagctcttctcaaagc

SEQ ID NO: 86 - DNA, 18 bp, synthetic construct
  source 1..18: mol_type = other DNA
gcacgggcagcttgccgg

SEQ ID NO: 87 - DNA, 20 bp, synthetic construct
  source 1..20: mol_type = other DNA
gctgaagcactgcacgccat

SEQ ID NO: 88 - DNA, 20 bp, synthetic construct
  source 1..20: mol_type = other DNA
aagtaaaacctctacaaatg

SEQ ID NO: 89 - DNA, 20 bp, synthetic construct
  source 1..20: mol_type = other DNA
tctttgaaagagcaataaaa

SEQ ID NO: 90 - DNA, 97 bp, synthetic construct
  source 1..97: mol_type = other DNA
aagttcatctgcaccaccggcaagctgccggtgccctggcccaccctcgtgaccaccctgacgtacggcgtgcagtgcttcagccgctaccccgacc

SEQ ID NO: 91 - DNA, 180 bp, synthetic construct
  source 1..180: mol_type = other DNA
tgatatgaatgaaactcatcaaatatgcgtgttagtgtaaatgaacttctatttaattttgaggctctgcaaagttctttaaaggagcagcagaatggcttcaactatctgagtgacactgtgaaggagatggccaagaaagcaccttcagaaatatgccagaaatatctgtcagaattt

(19) United States
(12) Patent Application Publication
(10) Pub. No.: US 2025/0127921 A1
(43) Pub. Date: Apr. 24, 2025

(54) PH-RESPONSIVE NANO PARTICLE FOR DELIVERY OF RIBONUCLEOPROTEINS

(71) Applicant: Wisconsin Alumni Research Foundation, Madison, WI (US)

(72) Inventors: Shaoqin Gong, Middleton, WI (US); Ruosen Xie, Madison, WI (US); Xiuxiu Wang, Madison, WI (US)

(73) Assignee: Wisconsin Alumni Research Foundation, Madison, WI (US)

(21) Appl. No.: 18/682,418

(22) PCT Filed: Aug. 9, 2022
(86) PCT No.: PCT/US2022/039879

§ 371 Date: Feb. 8, 2024

Related U.S. Application Data
(60) Provisional application No. 63/231,437, filed on Aug. 10, 2021.

(51) Int. Cl. A61K 47/69; A61K 9/51; A61K 31/7088; A61K 38/46; A61K 47/54; C12N 9/22; C12N 15/11; C12N 15/88

(52) U.S. Cl. CPC A61K 47/6935; A61K 9/5146; A61K 31/7088; A61K 38/465; A61K 47/545; C12N 9/22; C12N 15/11; C12N 15/88; C12N 2310/20; C12N 2800/80

(57) ABSTRACT

Provided herein are self-assembled nanoparticles (NPs), pharmaceutical compositions containing such NPs and methods of using such NPs. The NPs comprise an amphiphilic copolymer and a ribonucleoprotein (RNP), and optionally ssODN, wherein: the amphiphilic copolymer is a water-soluble block copolymer comprising a poly(C₂₋₃ alkylene glycol) block and an acrylic block comprising a poly(acrylate), poly(methacrylate) or poly(acrylate/methacrylate) block; the acrylic block comprise ester side chains bearing substituted or unsubstituted alkylamine groups; and the RNP, ssODN, and the acrylic block of the amphiphilic copolymer form a core of the self-assembled nanoparticle, and the poly(ethylene glycol) block of the amphiphilic copolymer forms the exterior of the self-assembled nanoparticle.

### CROSS REFERENCE TO RELATED APPLICATION

This application claims the benefit of and priority to U.S. Provisional Patent Application No. 63/231,437, filed on Aug. 10, 2021, the contents of which are incorporated herein by reference in its entirety.

### STATEMENT OF GOVERNMENT SUPPORT

This invention was made with government support under 1UG3NS111688-01 awarded by the National Institutes of Health. The government has certain rights in the invention.

### FIELD

The present technology relates generally to the field of nanoplatform delivery systems. The delivery systems include nanoparticles formed by self-assembly of ribonucleoproteins and amphiphilic copolymers. The copolymers include a polyethylene glycol block and a pH-responsive segment with ionizable amine groups which can deliver ribonucleoproteins (e.g., CRISPR ribonucleoproteins with or without a donor DNA template) to cells.

### BACKGROUND

Genome editing has demonstrated tremendous therapeutic potential to prevent and treat a wide range of pathological conditions over the last two decades. However, clinical translation has been limited due to various technical barriers, particularly, the lack of safe and efficient genome editing systems.

The clustered regularly interspaced short palindromic repeats (CRISPR)-Cas systems are powerful tools for genome editing. For example, the Cas9/sgRNA ribonucleoprotein (RNP) and the Cas12/crRNA RNP can knock out a target gene with high efficiency and specificity by inducing a double-strand DNA break at the target locus in the genome. Cells can repair such breaks by various means, including nonhomologous end joining (NHEJ), which makes small insertions or deletions to permanently silence the targeted gene. Moreover, the combination of RNP and single-stranded donor oligonucleotide (ssODN) used as the repair template can achieve precise genome editing to incorporate sequences from the ssODN (known as homology-directed repair or HDR). However, safe and efficient delivery of RNP and ssODN remains as a significant challenge for their potential application owing to their relatively large and complex structures. Unpackaged RNP and ssODN are also susceptible to chemical and/or enzymatic degradation. In comparison to DNA and mRNA delivery, the delivery of RNP and ssODN is even more challenging due to the mixed charges (e.g., positively charged Cas proteins and negatively charged sgRNA and ssODN) and more sophisticated structures.

### SUMMARY

The present technology provides self-assembled nanoparticles based on a pH-responsive amphiphilic polymer that can accommodate the heterogeneity (e.g., charge and hydrophobicity) of RNP, and optionally ssODN, via both electrostatic and hydrophobic interactions. The present nanoparticles (NPs) offer high loading efficiencies and small uniform sizes. The present technology may be used to prepare NHEJ-NP and HDR-NP that offer a safe and efficient approach for both in vitro and in vivo (via both local or systemic administration) genome editing and possess good biocompatibility and low immunogenicity.

Thus, in one aspect, the present technology provides self-assembled nanoparticles comprising an amphiphilic copolymer, a ribonucleoprotein (RNP), and optionally ssODN, wherein the amphiphilic copolymer is a water-soluble block copolymer comprising a poly(C₂₋₃ alkylene glycol) block and an acrylic block comprising a poly(acrylate), poly(methacrylate) or poly(acrylate/methacrylate) block; the acrylic block comprise ester side chains bearing substituted or unsubstituted alkylamine groups; and the RNP, ssODN, and the acrylic block of the amphiphilic copolymer form a core of the self-assembled nanoparticle, and the poly(ethylene glycol) block of the amphiphilic copolymer forms the exterior of the self-assembled nanoparticle.

In another aspect, the present technology provides pharmaceutical compositions comprising a self-assembled nanoparticle as described herein in a pharmaceutically acceptable carrier, e.g., a pharmaceutically acceptable aqueous carrier at a pH of 5-9.

In yet another aspect, the present technology provides methods of editing a targeted gene in a subject, the method comprising administering an effective amount of a self-assembled nanoparticle as described herein or a pharmaceutical composition thereof to the subject, whereby the RNP is targeted to the gene to be edited and edits the targeted gene.

The foregoing summary is illustrative only and is not intended to be in any way limiting. In addition to the illustrative aspects, embodiments and features described above, further aspects, embodiments and features will become apparent by reference to the following drawings and the detailed description.

### BRIEF DESCRIPTION OF THE DRAWINGS

FIGS. 1A-1I show schematic representations of illustrative embodiments of the present technology, and in particular, the design and characterization of an NHEJ-NP and an HDR-NP. 1A, The pH-sensitive mPEG-PC7A polymer forms nanoparticle (NP), through a self-assembly process, with either Cas9 RNP alone to enable genome editing via NHEJ (termed NHEJ-NP), or with both Cas9 RNP and ssODN (a donor DNA) to enable genome editing via HDR (termed HDR-NP). 1B, NPs are taken up by cells typically via an endocytosis process. The PC7A polymer segments become protonated in the acidic endosomal compartments, thereby triggering the disassembly of the NPs, and enabling the release and endosomal escape of the payload, and ultimately its delivery to the cytoplasm. Thereafter, the Cas9 RNP or Cas9 RNP with ssODN enters the nucleus, facilitated by NLS presented on Cas9, for genome editing. 1C, The hydrodynamic diameters of Cas9 RNP, ssODN, empty-NP, NHEJ-NP, and HDR-NP were measured by DLS. 1D, A representative TEM image of NHEJ-NP. Scale bar: 50 nm. 1E, An electrophoresis assay in 2% agarose gel for sgRNA, ssODN, Cas9 RNP, Cas9 RNP with ssODN, NHEJ-NP, and HDR-NP indicates efficient complexation between the payloads (i.e., Cas9 RNP for Cas9 RNP with ssODN) and mPEG-PC7A. 1F, Zeta-potentials of the Cas9 RNP, ssODN, empty-NP, NHEJ-NP, and HDR-NP indicate that the anionic charges of Cas9 RNP and ssODN were neutralized after being encapsulated inside the NPs. 1G, The sizes of NHEJ-NP dispersed in PBS, serum-containing cell culture medium, and serum (40 mg/mL bovine serum albumin in PBS) and stored at 4° C. and 37° C. were measured via DLS after 24 h. 1H, The size of NHEJ-NP varied in the PBS solution containing either Tween 20 or NaCl, but remained stable in the PBS solution or the PBS solution containing urea at 37° C. This study demonstrates that NHEJ-NP was formed by both hydrophobic and electrostatic interactions between the polymer and the Cas9 RNP. 1I, The hydrodynamic diameters of NHEJ-NP at different pH conditions were measured by DLS. Data are presented as mean±s.d. Statistical significance with the size of fresh NHEJ-NP was calculated via one-way ANOVA with Tukey's post hoc test. *P<0.05, **P<0.01, ****P<0.0001. ns, not significant. NLS, nuclear localization signal.

FIGS. 2A-2D show synthesis and ¹H-NMR characterization of the monomer and polymers. 2A, 2-(azepan-1-yl)ethyl methacrylate (C7A-MA); 2B, methoxy- or maleimide-poly(ethylene glycol)-bromide (mPEG-Br or Mal-PEG-Br); 2C, methoxy- or maleimide-poly(ethylene glycol-b-2-(azepan-1-yl)ethyl methacrylate) (mPEG-PC7A or Mal-PEG-PC7A); 2D, cell-penetrating peptide (CPP)-conjugated polymer (CPP-PEG-PC7A). Imp., impurities.

FIGS. 3A-3F show in vitro studies for NHEJ-NPs and HDR-NPs. 3A, Optimization of the pH value for preparation of NHEJ-NP for gene editing in GFP-expressing HEK293 cells. Four days after the treatment, the loss of GFP fluorescence was measured by flow cytometry to assay the editing efficiency via NHEJ. Data are presented as mean±s.d. (n=3). 3B, Optimization of the pH value for preparation of the HDR-NP for gene editing in BFP-expressing HEK293 cells. Gene correction via HDR (correcting CAT to TAC) leads to GFP expression, and gene disruption via NHEJ leads to silence of BFP expression. Four days after the treatment, the gain of GFP and loss of BFP fluorescence were measured by flow cytometry to assay the editing efficiency via HDR and NHEJ, respectively. The HDR efficiency and the HDR/NHEJ ratio are shown. Data are presented as mean±s.d. (n=3). 3C, HDR efficiency of cell-penetrating peptide-conjugated HDR-NPs (i.e., CPP-HDR-NP) in BFP-expressing H9 hESCs. Four days after the treatment, the gain of GFP fluorescence was measured by flow cytometry to assay the editing efficiency via HDR. Data are presented as mean±s.d. (n=3). 3D, Cell viability study of NHEJ-NP and HDR-NP in HEK293 cells via CCK-8 assay. Data are presented as mean±s.d. (n=3). 3E, Study of the endocytosis of NHEJ-NP and CPP-NHEJ-NP. HEK293 cells were first treated with various endocytosis inhibitors at 37° C. or were incubated at 4° C. Cas9 RNPs were labeled with Atto 550-gRNAs and then encapsulated in NHEJ-NP or CPP-NHEJ-NP to treat HEK293 cells. Four hours after treatments, the cellular uptake of Cas9 RNP was quantified by flow cytometry measuring Atto 550⁺ cells. Data are presented as mean±s.d. (n=4). 3F, Intracellular distribution of NHEJ-NP in HEK293 cells was observed by confocal laser scanning microscopy at different time points after treatments. The Cas9 RNP was labeled with Atto 550-gRNA (red). Cells were stained with LysoTracker Green DND-26 (green) and Hoechst 33342 (blue) for endosomes/lysosomes and nuclei, respectively. Experiments were repeated three times and representative images are shown. Scale bar: 50 μm. Data were analyzed by ImageJ. Statistical significance was calculated via one-way ANOVA with Tukey's post hoc test. *P<0.05, **P<0.01, ***P<0.001, ****P<0.0001. ns, not significant. n.d., not detected. “Lipo2000” represents Lipofectamine 2000. “CRISPRMAX” represents Lipofectamine CRISPRMAX. CPZ, chlorpromazine. MBCD, methyl-β-cyclodextrin.

FIGS. 4A-4D show optimization of the loading content for NHEJ-NP. 4A, Gene editing efficiency of NHEJ-NPs with different loading content of the Cas9 RNP in GFP-expressing HEK293 cells. Generally, formulations with a lower loading content (more polymers) led to higher editing efficiency. The loading contents for both NHEJ-NP and HDR-NP were set at 17% for in vitro and in vivo studies. 4B, Electrophoresis assay of NHEJ-NPs with different loading contents of Cas9 RNPs in 2% agarose gel. The result suggests that more polymers can lead to better encapsulation of the Cas9 RNP in the NPs, and thus lead to better editing efficiency. (4C) Hydrodynamic diameters and (4D) zeta-potentials of NHEJ-NPs with different loading contents of Cas9 RNPs indicate that nanoparticles were formed even with LC=50%. Therefore, the ineffectiveness of the formulations with high loading contents may also be attributed to their instability in the serum-containing cell culture media. Statistical significance was calculated via one-way ANOVA with Tukey's post hoc test. **P<0.01, ****P<0.0001. ns, not significant. Loading content (LC) is defined as:

$$LC = \frac{mass ( Cas 9 RNP )}{mass ( Cas 9 RNP ) + mass ( Polymer )} \times 100 \%$$

For example, “LC=17%” is equivalent to “Cas9 RNP/Polymer=⅕ (weight/weight)”.

FIGS. 5A-5F show in vivo gene editing with NHEJ-NP in Ai14 reporter mice via intravenous (i.v.) injections. 5A, Ai14 mice employ a STOP cassette that consists of three SV40 polyA sequences to prevent transcription of the downstream tdTomato, a red fluorescent protein. Successful gene editing with the Cas9 RNP that targets SV40 polyA sequences leads to excision of the STOP cassette and then the expression of tdTomato, although tdTomato activation requires removal of at least two of the three SV40 polyA repeat sequences and thus underreports the gene editing efficiency. 5B, Ai14 mice were i.v. injected with PBS (n=3) or NHEJ-NP (n=3) on Day 0, and the organs and tissues were collected on Day 7 for analysis. 5C, NHEJ-NP selectively edited and induced tdTomato expression in livers after intravenous injection, observed by IVIS. Images acquired from one PBS-injected and three NHEJ-NP-injected mice are presented. 5D, The percentages of tdTomato⁺ cells within defined cell type populations in liver were quantified by flow cytometry, where tdTomato expression was found in all major cell types in liver. Data are presented as mean±s.d. (n=3). 5E, 5F, Immunofluorescence staining of the liver sections from the PBS-injected and NHEJ-NP-injected mice suggests considerable gene editing in liver. Liver sections were stained with anti-RFP antibody for tdTomato (red), anti-hepatocyte specific antigen (green), anti-CD31 antibody for endothelial cells (magenta, pseudo-color in (5E)), anti-F4/80 antibody for Kupffer cells (magenta, pseudo-color in (5F)), and DAPI for nuclei (blue), respectively. Representative images are shown. Scale bar: 100 μm. ****P<0.0001. IVIS, in vivo imaging systems.

FIGS. 6A-6D show The NHEJ efficiency and HDR efficiency induced by HDR-NPs in BFP-expressing HEK293 cells. 6A, Percentages of cells edited via HDR, NHEJ, or unedited cells, respectively, after 96 h incubation with HDR-NPs formed at different pH values. 6B-6C, The respective (6B) NHEJ efficiency and (6C) percentage of unedited cells after treatments. Data were collected and analyzed by flow cytometry. Data are presented as mean±s.d. (n=3). 6D, Normalized HDR efficiency by HDR-NPs formed by different Cas9 RNP/ssODN molar ratios in BFP-expressing HEK293 cells. Statistical significance was calculated via one-way ANOVA with Tukey's post hoc test. *P<0.05, ****P<0.0001. ns, not significant.

FIGS. 7A-7C show the Pearson correlation coefficient and Mander's colocalization coefficient used to analyze the colocalization of the intracellular fluorescence signals of RNP and those of endosomes or nuclei in HEK293 cells. (7A) The Pearson correlation coefficient ranges from −1 to +1, with −1 for anti-correlation, 0 for no correlation, and +1 for perfect correlation. The data suggests the correlations of signals between RNPs and endosomes and those between RNPs and nuclei increased with longer incubation time, indicating increasing cellular uptake and nuclear transport of RNPs. (7B, 7C) The Mander's colocalization coefficient (MCC) ranges from 0 to 1, quantifying the fraction of intensity of one color that is co-localized with the other color in the same pixel of the image. (7B) The MCC suggests more RNPs were distributed in endosomes than those in nuclei at 0.5 and 2 h post-treatment, but more RNPs were colocalized with nuclei at 4 h post-treatment, indicating effective endosomal escape and nuclear transport of RNPs. (7C) The MCC suggests that the overlap between signals from endosomes or nuclei and signals from RNP increased with incubation time, as the result of continuous cellular uptake and nuclear transport of RNPs. The MCCs of (7B) and (7C) are defined as:

$$for ( b ) , MCC = \frac{\sum RNP ( signals colocalized with endosomes or nuclef )}{\sum RNP ( all signals )}$$

$$for ( c ) , MCC = \frac{\sum endosomes or nuclef ( signals colocalized with RNP )}{\sum endosomes or nuclef ( all signals )}$$

FIGS. 8A-8B show additional liver data from Ai14 mice i.v. injected with NHEJ-NP. 8A, IVIS images acquired from the other two PBS-injected mice are presented, supplementary to FIG. 3C. 8B, The fluorescence intensities of the homogenized suspension from livers of the PBS-injected and NHEJ-NP-injected mice. The tdTomato fluorescence intensity was 2-fold higher than the PBS-injected group. Data are presented as mean±s.d. (n=3). Statistical significance was calculated via a t-test. ****P<0.0001. IVIS, in vivo imaging systems.

FIGS. 9A-9D show in vivo gene editing with NHEJ-NP in Ai14 reporter mice via intramuscular (i.m.) injections. 9A, The Ai14 mouse was i.m. injected with NHEJ-NP in the right side of tibialis anterior (TA) muscle on Day 0. The left side of TA muscle was injected with PBS as control. The muscles were collected on Day 7 for analysis. 9B, NHEJ-NP efficiently edited and induced tdTomato expression in TA muscles after injections. tdTomato fluorescence in TA muscles was measured by IVIS. Images acquired with two magnifications from both the PBS-injected and NHEJ-NP-injected mice are presented (n=3). Scale bar: 500 μm for 4× images and 100 μm for 20× images. 9c, Immunofluorescence staining of the muscle sections from the PBS-injected and NHEJ-NP-injected mice. The muscle sections were stained with anti-RFP antibodies for tdTomato (red) and DAPI for nuclei (blue), respectively. Experiments were repeated three times, and representative images with two magnifications are shown. 9d, Gene editing efficiency was quantified by the percentage area of the whole muscle section with a genome editing reporter (tdTomato+) or nuclei (control), analyzed by ImageJ. **P<0.01. ns, not significant. IVIS, in vivo imaging systems.

FIGS. 10A-10I show in vivo gene editing with HDR-NP via intramuscular (i.m.) injections for Duchenne muscular dystrophy treatment. 10A, The mdx mouse was i.m. injected with HDR-NP in triceps brachii, gastrocnemius, and tibialis anterior muscles on Day 0, respectively. Muscles were harvested on Day 28 for analysis. 10B, The four-limb hanging time assay of the PBS-injected mdx mice (n=7, negative control), HDR-NP-injected mdx mice (n=11), and untreated wild-type mice (n=6, positive control) demonstrates the restoration of muscle strength of mdx mice after HDR-NP-mediated gene editing. Data were acquired 28 days after treatments. 10C and 10D, The gene editing efficiency through (c) NHEJ and (d) HDR induced by HDR-NP in the tibialis anterior muscle assayed by Sanger sequencing. Data were acquired 7 days after treatments and presented as mean±s.d. (n=3). 10E, Immunogenicity of PBS (as reference) or HDR-NP injection in tibialis anterior muscles of mdx mice. Expression levels of cytokines were quantified by RT-PCR. 10F-H, Immunofluorescence staining of (f) triceps brachii, (g) gastrocnemius, and (h) tibialis anterior muscle sections from the PBS-injected and HDR-NP-injected mdx mice and untreated wild-type mice. Muscle sections were stained with anti-dystrophin antibodies for dystrophin (red) and DAPI for nuclei (blue), respectively. Results indicate robust restoration of dystrophin expression after HDR-NP treatments. Representative images are presented. Scale bar: 100 μm. 10G, The histology of muscle sections was investigated by Masson's trichrome staining. Reduced muscular dystrophy was observed in HDR-NP-treated mice. Scale bar: 100 μm. Statistical significance was calculated via (b-d) one-way ANOVA or (e) two-way ANOVA with Tukey's post hoc test. *P<0.05, ***P<0.001, ****P<0.0001. ns, not significant. NC RNP, Cas9 RNP with negative control sgRNA. Dmd RNP, Cas9 RNP with sgRNA that targets Dmd exon 23.

FIGS. 11A-11D show change of hanging times and body weights for mdx mice with or without HDR-NP treatment. 11A-11C, The four-limb hanging time assay of the PBS-injected mdx mice (n=7, negative control), HDR-NP-injected mdx mice (n=11), and untreated wild-type mice (n=6, positive control) demonstrates the restoration of muscle strength of mdx mice (11A) 2 weeks and (11B) 3 weeks after injections with HDR-NPs. (c) The hanging time changes for all tested mice are summarized. 11D, The body weights of PBS-injected and HDR-NP-injected mdx mice. Data are presented as mean±s.d. Statistical significance was calculated via (11A, 11B) one-way or (11C, 11D) two-way ANOVA with Tukey's post hoc test. *P<0.05, **P<0.01, ***P<0.001, ****P<0.001. ns, not significant.

FIGS. 12A-12B show the blood biochemical parameter panel for NHEJ-NP injected Ai14 mice. Fresh whole blood was collected from Ai14 mice on Day 7. No significant variations in these biochemical parameters were found in animals treated with NHEJ-NPs via both administration routes. ALT, alanine aminotransferase. AST, aspartate aminotransferase. AST/ALT, the ratio of AST/ALT. ALP, alkaline phosphatase. BUN, blood urea nitrogen. CRE, creatinine. TBIL, total bilirubin. GLU, glucose. Ca²⁺, total calcium. TP, total protein. ALB, albumin. GLOB, globulin. Na⁺, sodium. K⁺, potassium. Cl⁻, chloride. tCO₂, total carbon dioxide. Statistical significance was calculated with PBS-injected as the control via one-way ANOVA with Tukey's post hoc test. *P<0.05. ns, not significant.

FIG. 13 shows H&E staining for major organs and TA muscles from PBS-injected and NHEJ-NP injected Ai14 mice. No pathological changes were found in the mouse major organs and muscles treated with NHEJ-NPs through both i.v. and i.m. injections. TA, tibialis anterior. i.v., intravenous. i.m., intramuscular. Scale bar: 100 μm.

FIG. 14 shows the stability and storability of NHEJ-NP. NHEJ-NP was stored in Tris-EDTA buffer (pH 7.5) with 8% (v/v) glycerol at different temperatures for certain durations and then was used to treat BFP-HEK 293 cells. The editing efficiency was assayed by flow cytometry to quantify BFP-negative cells. Data are presented as mean±s.d. (n=3). Data are presented as mean±s.d. Statistical significance was calculated using “Fresh” or “Day 0” as the control via one-way ANOVA with Tukey's post hoc test. ns, not significant.

FIGS. 15A-15D show in vivo gene editing in Ai14 mice by intranasally (i.n.) injection of NHEJ-NP encapsulating Cas9 RNP. 15A, Ai14 mice were i.n. injected with PBS (n=3) or NHEJ-NP encapsulating Cas9 RNP (n=3) on Day 0, respectively. The lungs were collected on Day 14 for analysis. 15B, NHEJ-NP effectively edited the lung and induced tdTomato expression after i.n. injection according to the IVIS analysis. Images acquired from three PBS-injected and three NHEJ-NP (Cas9 RNP)-injected mice are presented. 15C, The tdTomato fluorescence intensity of NHEJ-NP (Cas9 RNP)-injected lungs was significantly higher than the PBS-injected ones, as determined by IVIS. Data are presented as mean±s.d. (n=3). Statistical significance was calculated via Student's t-test. *P<0.05. 15D, Immunofluorescence staining of the lung sections from the PBS-treated and NHEJ-NP-treated mice suggests effective gene editing in lung. Lung sections were stained with anti-RFP antibody for tdTomato (red, 2^{nd} col.), anti-Ep-CAM antibody for epithelia cells (green, 3^{rd} col.), and DAPI for nuclei (blue, 4^{th} col.), respectively. Representative images are shown. Scale bar: 200 μm.

FIGS. 16A-16D show in vivo gene editing in Ai14 mice by intratracheal (i.t.) injections of NHEJ-NP. 16A, Ai14 mice were i.t. injected with PBS (n=3), isotonic NHEJ-NP (n=3), and hypotonic NHEJ-NP (n=3) on Day 0, and the organs and tissues were collected on Day 14 for analysis. 16B, NHEJ-NP induced tdTomato expression in lung after intratracheal injections, observed by IVIS. Images acquired from all mice are presented. 16C, The total flux of tdTomato fluorescence in lungs were measured. Significant tdTomato fluorescence enhancement was achieved after gene editing. 16D, Immunofluorescence staining of the lung sections from the PBS-injected and NHEJ-NP-injected mice suggests efficacious gene editing in lungs. Sections were stained with anti-RFP antibody for tdTomato (red), anti-EpCAM antibody for airway epithelial cells (green), and DAPI for nuclei (blue), respectively. Representative images are shown. Scale bars: 200 μm (10×) and 50 μm (40×). Statistical significance was calculated via two-way ANOVA with Tukey's post hoc test. **P<0.01, ****P<0.0001

### DETAILED DESCRIPTION

The following terms are used throughout as defined below. All other terms and phrases used herein have their ordinary meanings as one of skill in the art would understand.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In any embodiments, a substituted group is substituted with 1, 2, 3, 4, 5, or 6 substituents. Examples of substituent groups include: halogens (i.e., F, Cl, Br, and I); hydroxyls; alkoxy, alkenoxy, aryloxy, aralkyloxy, heterocyclyl, heterocyclylalkyl, heterocyclyloxy, and heterocyclylalkoxy groups; carbonyls (oxo); carboxylates; esters; urethanes; oximes; hydroxylamines; alkoxyamines; aralkoxyamines; thiols; sulfides; sulfoxides; sulfones; sulfonyls; sulfonamides; sulfates; phosphates; amines; N-oxides; hydrazines; hydrazides; hydrazones; azides (—N₃); amides; ureas; amidines; guanidines; enamines; imides; imines; nitro groups (—NO₂); nitriles (—CN); and the like.

Substituted ring groups such as substituted cycloalkyl, aryl, heterocyclyl and heteroaryl groups also include rings and ring systems in which a bond to a hydrogen atom is replaced with a bond to a carbon atom. Therefore, substituted cycloalkyl, aryl, heterocyclyl and heteroaryl groups may also be substituted with substituted or unsubstituted alkyl, alkenyl, and alkynyl groups as defined below.

Alkyl groups include straight chain and branched chain alkyl groups having (unless indicated otherwise) from 1 to 12 carbon atoms, and typically from 1 to 10 carbons or, in any embodiments, from 1 to 8, 1 to 6, or 1 to 4 carbon atoms. Alkyl groups may be substituted or unsubstituted. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above, and include without limitation haloalkyl (e.g., trifluoromethyl), hydroxyalkyl, thioalkyl, aminoalkyl, alkylaminoalkyl, dialkylaminoalkyl, amidinealkyl, guanidinealkyl, alkoxyalkyl, carboxyalkyl, and the like.

Alkenyl groups include straight and branched chain alkyl groups as defined above, except that at least one double bond exists between two carbon atoms. Alkenyl groups may be substituted or unsubstituted. Alkenyl groups have from 2 to 12 carbon atoms, and typically from 2 to 10 carbons or, in any embodiments, from 2 to 8, 2 to 6, or 2 to 4 carbon atoms. In any embodiments, the alkenyl group has one, two, or three carbon-carbon double bonds. Examples include, but are not limited to vinyl, allyl, —CH═CH(CH₃), —CH═C(CH₃)₂, —C(CH₃)═CH₂, —C(CH₃)═CH(CH₃), —C(CH₂CH₃)═CH₂, among others. Representative substituted alkenyl groups may be mono-substituted or substituted more than once, such as, but not limited to, mono-, di- or tri-substituted with substituents such as those listed above for alkyl.

Aryl groups are cyclic aromatic hydrocarbons that do not contain heteroatoms. Aryl groups herein include monocyclic, bicyclic and tricyclic ring systems. Aryl groups may be substituted or unsubstituted. Thus, aryl groups include, but are not limited to, phenyl, azulenyl, heptalenyl, biphenyl, fluorenyl, phenanthrenyl, anthracenyl, indenyl, indanyl, pentalenyl, and naphthyl groups. In any embodiments, aryl groups contain 6-14 carbons, and in others from 6 to 12 or even 6-10 carbon atoms in the ring portions of the groups. In any embodiments, the aryl groups are phenyl or naphthyl. The phrase “aryl groups” includes groups containing fused rings, such as fused aromatic-aliphatic ring systems (e.g., indanyl, tetrahydronaphthyl, and the like). Representative substituted aryl groups may be mono-substituted (e.g., tolyl) or substituted more than once. For example, monosubstituted aryl groups include, but are not limited to, 2-, 3-, 4-, 5-, or 6-substituted phenyl or naphthyl groups, which may be substituted with substituents such as those listed above.

Aralkyl groups are alkyl groups as defined above in which a hydrogen or carbon bond of an alkyl group is replaced with a bond to an aryl group as defined above. Aralkyl groups may be substituted or unsubstituted. In any embodiments, aralkyl groups contain 7 to 16 carbon atoms, 7 to 14 carbon atoms, or 7 to 10 carbon atoms. Substituted aralkyl groups may be substituted at the alkyl, the aryl or both the alkyl and aryl portions of the group. Representative aralkyl groups include but are not limited to benzyl and phenethyl groups and fused (cycloalkylaryl)alkyl groups such as 4-indanylethyl. Representative substituted aralkyl groups may be substituted one or more times with substituents such as those listed above.

Heteroalkyl groups include alkyl groups as defined above in which one to five of the carbon atoms are replaced by a heteroatom selected from N, O, and S, provided that no more than two heteroatoms are adjacent to each other. In any embodiments, the heteroalkyl group may have 1, 2, 3, 4 or 5 heteroatoms or a range between and including any two of the foregoing values such as from 1-5, 1-4, 1-3, 1-2, 2-5, 2-4, 2-3, 3-5, or 3-4. For example, in any embodiments the heteroalkyl group includes 1-5 O atoms. In any embodiments the heteroalkyl group includes only O atoms. In any embodiments the heteroalkyl group includes only N atoms or N and O atoms. When the heteroalkyl includes N, the N may be a primary N, secondary N, or a tertiary N. For example, the N atom may be a terminal N (e.g., NH₂), an N atom within an alkyl chain (e.g., NH), or be bound to 3 alkyl groups (e.g., —CH₂CH₂—N(CH₃)(CH₂CH₂CH₃)). In any embodiments, the heteroalkyl group includes only S atoms, e.g., 1-2 S atoms.

Heterocyclyl groups include aromatic (also referred to as heteroaryl) and non-aromatic carbon-containing ring compounds containing 3 or more ring members, of which one or more is a heteroatom such as, but not limited to, N, O, and S. In any embodiments, the heterocyclyl group contains 1, 2, 3 or 4 heteroatoms. In any embodiments, heterocyclyl groups include mono-, bi- and tricyclic rings having 3 to 16 ring members, whereas other such groups have 3 to 6, 3 to 10, 3 to 12, or 3 to 14 ring members. Heterocyclyl groups encompass aromatic, partially unsaturated and saturated ring systems, such as, for example, imidazolyl, imidazolinyl and imidazolidinyl groups. The phrase “heterocyclyl group” includes fused ring species including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. However, the phrase does not include heterocyclyl groups that have other groups, such as alkyl, oxo or halo groups, bonded to one of the ring members. Rather, these are referred to as “substituted heterocyclyl groups”. Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, oxadiazolonyl (including 1,2,4-oxazol-5(4H)-one-3-yl), isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed above.

Heteroaryl groups are aromatic carbon-containing ring compounds containing 5 or more ring members, of which, one or more is a heteroatom such as, but not limited to, N, O, and S. Heteroaryl groups include, but are not limited to, groups such as pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl (pyrrolopyridinyl), indazolyl, benzimidazolyl, imidazopyridinyl (azabenzimidazolyl), pyrazolopyridinyl, triazolopyridinyl, benzotriazolyl, benzoxazolyl, benzothiazolyl, benzothiadiazolyl, imidazopyridinyl, isoxazolopyridinyl, thianaphthyl, purinyl, xanthinyl, adeninyl, guaninyl, quinolinyl, isoquinolinyl, tetrahydroquinolinyl, quinoxalinyl, and quinazolinyl groups. Heteroaryl groups include fused ring compounds in which all rings are aromatic such as indolyl groups and include fused ring compounds in which only one of the rings is aromatic, such as 2,3-dihydro indolyl groups. Although the phrase “heteroaryl groups” includes fused ring compounds, the phrase does not include heteroaryl groups that have other groups bonded to one of the ring members, such as alkyl groups. Rather, heteroaryl groups with such substitution are referred to as “substituted heteroaryl groups.” Representative substituted heteroaryl groups may be substituted one or more times with various substituents such as those listed above.

Heterocyclylalkyl groups are alkyl groups as defined above in which a hydrogen or carbon bond of an alkyl group is replaced with a bond to a heterocyclyl group as defined above. Substituted heterocyclylalkyl groups may be substituted at the alkyl, the heterocyclyl or both the alkyl and heterocyclyl portions of the group. Representative heterocyclyl alkyl groups include, but are not limited to, morpholin-4-yl-ethyl, furan-2-yl-methyl, imidazol-4-yl-methyl, pyridin-3-yl-methyl, tetrahydrofuran-2-yl-ethyl, and indol-2-yl-propyl. Representative substituted heterocyclylalkyl groups may be substituted one or more times with substituents such as those listed above.

Heteroaralkyl groups are alkyl groups as defined above in which a hydrogen or carbon bond of an alkyl group is replaced with a bond to a heteroaryl group as defined above. Substituted heteroaralkyl groups may be substituted at the alkyl, the heteroaryl or both the alkyl and heteroaryl portions of the group. Representative substituted heteroaralkyl groups may be substituted one or more times with substituents such as those listed above.

Groups described herein having two or more points of attachment (i.e., divalent, trivalent, or polyvalent) within the compound of the present technology are designated by use of the suffix, “ene.” For example, divalent alkyl groups are alkylene groups, divalent alkenyl groups are alkenylene groups, and so forth. Substituted groups having a single point of attachment to a compound or polymer of the present technology are not referred to using the “ene” designation. Thus, e.g., chloroethyl is not referred to herein as chloroethylene.

Alkoxy groups are hydroxyl groups (—OH) in which the bond to the hydrogen atom is replaced by a bond to a carbon atom of a substituted or unsubstituted alkyl group as defined above. Alkoxy groups may be substituted or unsubstituted. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Examples of cycloalkoxy groups include but are not limited to cyclopropyloxy, cyclobutyloxy, cyclopentyloxy, cyclohexyloxy, and the like. Representative substituted alkoxy groups may be substituted one or more times with substituents such as those listed above.

The term “amide” (or “amido”) includes C- and N-amide groups, i.e., —C(O)NR⁷¹R⁷², and —NR⁷¹C(O)R⁷² groups, respectively. R⁷¹ and R⁷² are independently hydrogen, or a substituted or unsubstituted alkyl, alkenyl, cycloalkyl, aryl, aralkyl, heterocyclylalkyl or heterocyclyl group as defined herein. Amido groups therefore include but are not limited to carbamoyl groups (—C(O)NH₂) (also referred to as “carboxamido groups”) and formamido groups (—NHC(O)H). In any embodiments, the amide is —NR⁷¹C(O)—(C₁₋₅ alkyl) and the group is termed “alkanoylamino.”

The term “amine” (or “amino”) as used herein refers to —NR⁷⁵R⁷⁶ groups, wherein R⁷⁵ and R⁷⁶ are independently hydrogen, or a substituted or unsubstituted alkyl, alkenyl, cycloalkyl, aryl, aralkyl, heterocyclylalkyl or heterocyclyl group as defined herein. In any embodiments, the amine is NH₂, alkylamino, dialkylamino, arylamino, or alkylarylamino. In other embodiments, the amine is NH₂, methylamino, dimethylamino, ethylamino, diethylamino, propylamino, isopropylamino, phenylamino, or benzylamino. It will be understood that amines may exist in protonated forms in certain aqueous solutions or mixtures and are examples of charged functional groups herein.

The term “carboxyl” or “carboxylate” as used herein refers to a —COOH group or its ionized salt form. As such, it will be understood that carboxyl groups are examples of charged functional groups herein.

The term “ester” as used herein refers to —COOR⁷⁰ and —C(O)O-G groups. R⁷⁰ is a substituted or unsubstituted alkyl, cycloalkyl, alkenyl, aryl, aralkyl, heterocyclylalkyl or heterocyclyl group as defined herein. G is a carboxylate protecting group. As used herein, the term “protecting group” refers to a chemical group that exhibits the following characteristics: 1) reacts selectively with the desired functionality in good yield to give a protected substrate that is stable to the projected reactions for which protection is desired; 2) is selectively removable from the protected substrate to yield the desired functionality; and 3) is removable in good yield by reagents compatible with the other functional group(s) present or generated in such projected reactions. Carboxylate protecting groups are well known to one of ordinary skill in the art. An extensive list of protecting groups for the carboxylate group functionality may be found in Protective Groups in Organic Synthesis, Greene, T. W.; Wuts, P. G. M., John Wiley & Sons, New York, NY, (3^{rd} Edition, 1999). Which can be added or removed using the procedures set forth therein and which is hereby incorporated by reference in its entirety and for any and all purposes as if fully set forth herein.

The term “guanidine” refers to —NR⁹⁰C(NR⁹¹)NR⁹²R⁹³, wherein R⁹⁰, R⁹¹, R⁹² and R⁹³ are each independently hydrogen, or a substituted or unsubstituted alkyl, cycloalkyl, alkenyl, aryl aralkyl, heterocyclyl or heterocyclylalkyl group as defined herein. It will be understood that guanidines may exist in protonated forms in certain aqueous solutions or mixtures and are examples of charged functional groups herein.

The term “hydroxyl” as used herein can refer to —OH or its ionized form, —O⁻. A “hydroxyalkyl” group is a hydroxyl-substituted alkyl group, such as HO—CH₂—.

The term “imidazolyl” as used herein refers to an imidazole group or the salt thereof. An imidazolyl may be protonated in certain aqueous solutions or mixtures, and is then termed an “imidazolate.”

The term “phosphate” as used herein refers to —OPO₃H₂ or any of its ionized salt forms, —OPO₃HR⁸⁴ or —OPO₃R⁸⁴R⁸⁵ wherein R⁸⁴ and R⁸⁵ are independently a positive counterion, e.g., Na⁺, K⁺, ammonium, etc. As such, it will be understood that phosphates are examples of charged functional groups herein.

The term “pyridinyl” refers to a pyridine group or a salt thereof. A pyridinyl may be protonated in certain aqueous solutions or mixtures, and is then termed a “pyridinium group”.

The term “sulfate” as used herein refers to —OSO₃H or its ionized salt form, —OSO₃R⁸⁶ wherein R⁸⁶ is a positive counterion, e.g., Na⁺, K⁺, ammonium, etc. As such, it will be understood that sulfates are examples of charged functional groups herein.

The term “thiol” refers to —SH groups, while “sulfides” include —SR⁸⁰ groups, “sulfoxides” include —S(O)R⁸¹ groups, “sulfones” include —SO₂R⁸² groups, and “sulfonyls” include —SO₂OR⁸³. R⁸⁰, R⁸¹, and R⁸² are each independently a substituted or unsubstituted alkyl, cycloalkyl, alkenyl, aryl aralkyl, heterocyclyl or heterocyclylalkyl group as defined herein. In any embodiments the sulfide is an alkylthio group, —S-alkyl. R⁸³ includes H or, when the sulfonyl is ionized (i.e., as a sulfonate), a positive counterion, e.g., Na*, K⁺, ammonium or the like. As such, it will be understood that sulfonyls are examples of charged functional groups herein.

Urethane groups include N- and O-urethane groups, i.e., —NR⁷³C(O)OR⁷⁴ and —OC(O)NR⁷³R⁷⁴ groups, respectively. R⁷³ and R⁷⁴ are independently a substituted or unsubstituted alkyl, alkenyl, alkynyl, cycloalkyl, aryl, aralkyl, heterocyclylalkyl, or heterocyclyl group as defined herein. R⁷³ may also be H.

“Treating” within the context of the instant technology, means alleviation, in whole or in part, of symptoms associated with a disorder or disease, or slowing, inhibition or halting of further progression or worsening of those symptoms, or prevention or prophylaxis of the disease or disorder in a subject at risk for developing the disease or disorder. For example, within the context of treating fungal infections, successful treatment may include reduction or eradication of the pathogenic fungus, from the body; clinical benefit; an alleviation of symptoms, such as a reduction or elimination of rash, itching, chafing, burning, throat thrush, redness, soreness, fever, cough, night sweats, weight loss, wheezing, and shortness of breath.

As used herein, an “effective amount” of a compound of the present technology refers to an amount of the compound that alleviates, in whole or in part, symptoms associated with a disorder or disease, or slows or halts of further progression or worsening of those symptoms, or prevents or provides prophylaxis for the disease or disorder in a subject at risk for developing the disease or disorder. Those skilled in the art are readily able to determine an effective amount. For example, one way of assessing an effective amount for a particular disease state is by simply administering an NP or composition of the present technology to a subject in increasing amounts until progression of the disease state is decreased or stopped or reversed.

Those of skill in the art will appreciate that compounds of the present technology may exhibit the phenomena of tautomerism, conformational isomerism, geometric isomerism and/or stereoisomerism. As the formula drawings within the specification and claims can represent only one of the possible tautomeric, conformational isomeric, stereoisomeric or geometric isomeric forms, it should be understood that the technology encompasses any tautomeric, conformational isomeric, stereoisomeric and/or geometric isomeric forms of the compounds having one or more of the utilities described herein, as well as mixtures of these various different forms.

Stereoisomers of compounds (also known as optical isomers) include all chiral, diastereomeric, and racemic forms of a structure, unless the specific stereochemistry is expressly indicated. Thus, compounds disclosed herein include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these stereoisomers are all within the scope of the present technology.

“Tautomers” refers to isomeric forms of a compound that are in equilibrium with each other. The presence and concentrations of the isomeric forms will depend on the environment the compound is found in and may be different depending upon, for example, whether the compound is a solid or is in an organic or aqueous solution. For example, in aqueous solution, imines may exhibit the following isomeric forms, which are referred to as tautomers of each other:

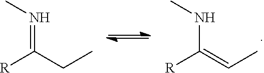

Because of the limits of representing compounds by structural formulas, it is to be understood that all chemical formulas of the compounds described herein represent all tautomeric forms of compounds and are within the scope of the present technology.

As used herein, the term “protecting group” refers to a chemical group that exhibits the following characteristics: 1) reacts selectively with the desired functionality in good yield to give a protected substrate that is stable to the projected reactions for which protection is desired; 2) is selectively removable from the protected substrate to yield the desired functionality; and 3) is removable in good yield by reagents compatible with the other functional group(s) present or generated in such projected reactions. Examples of suitable protecting groups can be found in Greene et al. (1991) Protective Groups in Organic Synthesis, 3rd Ed. (John Wiley & Sons, Inc., New York). Amino protecting groups include, but are not limited to, mesitylenesulfonyl (Mts), benzyloxycarbonyl (Cbz or Z), t-butyloxycarbonyl (Boc), t-butyldimethylsilyl (TBS or TBDMS), 9-fluorenylmethyloxycarbonyl (Fmoc), allyloxycarbonyl (Alloc), tosyl, benzenesulfonyl, 2-pyridyl sulfonyl, or suitable photolabile protecting groups such as 6-nitroveratryloxy carbonyl (Nvoc), nitropiperonyl, pyrenylmethoxycarbonyl, nitrobenzyl, α,α-dimethyldimethoxybenzyloxycarbonyl (DDZ), 5-bromo-7-nitroindolinyl, and the like. Amino protecting groups susceptible to acid-mediated removal include but are not limited to Boc and TBDMS. Amino protecting groups resistant to acid-mediated removal and susceptible to hydrogen-mediated removal include but are not limited to Alloc, Cbz, nitro, and 2-chlorobenzyloxycarbonyl.

As used herein, “ribonucleoprotein” or “RNP” refers to a complex between an RNA-binding protein and RNA in which the RNA binds specifically (as opposed to non-specific binding) to the protein. Examples of ribonucleoproteins include CRISPR-associated proteins, e.g., Cas9, Cas12, Cas 13, Cas14 and CasΦ.

As used herein, “Cas9” refers to the complex of Cas9 proteins and variants thereof having nuclease activity, with RNA (i.e., sgRNA or crRNA and tracrRNA). Likewise, “Cas12” refers to the complex of Cas12 proteins and variants thereof having nuclease activity, with crRNA. “Cas13” refers to the complex of Cas13 proteins and variants thereof having nuclease activity, with RNA (i.e., crRNA). Cas9, Cas12, and Cas13 also include complexes of fusion proteins containing such Cas9, Cas12, and Cas13 proteins and variants thereof. The fused proteins may include those that modify the epigenome or control transcriptional activity. The variants may include deletions or additions, such as, e.g., addition of one, two, or more nuclear localization sequences (such as from SV40 and others known in the art), e.g., 1, 2, 3, 4, 5, 6, 7, 8, 9, 10 such sequences or a range between and including any two of the foregoing values. In any embodiments the Cas9 polypeptide is a Cas9 protein found in a type II CRISPR-associated system. Suitable Cas9 polypeptides that may be used in the present technology include, but are not limited to Cas9 protein from Streptococcus pyogenes (SpCas9), F. novicida (FnCas9), S. aureus (SaCas9), S. thermophiles (St1Cas9), N. meningitidis (NmeCas9), and variants thereof. In any embodiments, the Cas9 polypeptide is a wild-type Cas9, a nickase, or comprises a nuclease inactivated (dCas9) protein. In any embodiments the Cas12 polypeptide is a Cas12 protein found in a type V CRISPR-associated system. Suitable Cas12 polypeptides that may be used in the present technology include, but are not limited to Cas12 protein from Lachnospiraceae bacterium MA2020 (LbCas12a), Acidaminococcus sp. BV3L6 (AsCas12a), Bacillus hisaishi (BhCas12b), and variants thereof. In any embodiments, the Cas12 polypeptide is a wild-type Cas12, a nickase, or comprises a nuclease inactivated (dCas12) protein. In any embodiments the Cas13 polypeptide is a Cas13 protein found in a type VI CRISPR-associated system. Suitable Cas13 polypeptides that may be used in the present technology include, but are not limited to Cas13 protein from Leptotrichia wadei (LwaCas13a), Prevotella sp. P5-125 (PspCas13b), Ruminococcus flavefaciens (RfxCas13d), and variants thereof. In any embodiments, the Cas13 polypeptide is a wild-type Cas13, a nickase, or comprises a nuclease inactivated (dCas13) protein. In any embodiments, the Cas9 polypeptide is a fusion protein comprising dCas9. In any embodiments, the Cas12 polypeptide is a fusion protein comprising dCas12. In any embodiments, the Cas13 polypeptide is a fusion protein comprising dCas13. In any embodiments, the fusion protein comprises a transcriptional activator (e.g., VP64), a transcriptional repressor (e.g., KRAB, SID) a nuclease domain (e.g., FokI), base editor (e.g., adenine base editors, ABE), a recombinase domain (e.g., Hin, Gin, or Tn3), a deaminase (e.g., a cytidine deaminase or an adenosine deaminase) or an epigenetic modifier domain (e.g., TET1, p300). In any embodiments, the Cas9, Cas12, or Cas13 includes variants with at least 85% sequence identity, at least 90% sequence identity, at least 95% sequence identity, or even 96%, 97%, 98%, or 99% sequence identity to the wild type Cas9 or Cas12, respectively. Accordingly, a wide variety of Cas9, Cas12, and Cas13 proteins may be used as formation of the present NPs is not sequence dependent so long as the Cas9 protein or Cas12 protein can complex with nucleic acids and the resulting RNP has sufficient charged residuals to allow complexation with the amphiphilic polymers of the present technology. Other suitable Cas9 proteins may be found in Karvelis, G. et al. “Harnessing the natural diversity and in vitro evolution of Cas9 to expand the genome editing toolbox,” Current Opinion in Microbiology 37: 88-94 (2017); Komor, A. C. et al. “CRISPR-Based Technologies for the Manipulation of Eukaryotic Genomes,” Cell 168:20-36 (2017); and Murovec, J. et al. “New variants of CRISPR RNA-guided genome editing enzymes,” Plant Biotechnol. J. 15:917-26 (2017), each of which is incorporated by reference herein in their entirety. Other suitable Cas12 proteins may be found in Makarova, Kira S., et al. “Evolutionary classification of CRISPR-Cas systems: a burst of class 2 and derived variants.” Nature Reviews Microbiology 18.2 (2020): 67-83; Strecker, Jonathan, et al. “Engineering of CRISPR-Cas12b for human genome editing.” Nature Comm. 10.1 (2019): 1-8; and Yan, Winston X., et al. “Functionally diverse type V CRISPR-Cas systems.” Science 363.6422 (2019): 88-91, each of which is incorporated by reference herein in their entirety. Other suitable Cas13 proteins may be found in O'Connell, Mitchell R. “Molecular mechanisms of RNA targeting by Cas13-containing type VI CRISPR-Cas systems.” J. Mol. Biol. 431.1 (2019): 66-87, each of which is incorporated by reference herein in their entirety.

As described herein, cell-penetrating peptides may be attached directly or indirectly to the terminus of the PEG block of the amphiphilic polymer. A “cell-penetrating peptide” (CPP), also referred to as a “protein transduction domain” (PTD), a “membrane translocating sequence,” and a “Trojan peptide”, refers to a short peptide (e.g., from 3 to about 40 amino acids) that has the ability to translocate across a cellular membrane to gain access to the interior of a cell and to carry into the cells a variety of covalently and noncovalently conjugated cargoes, including proteins, oligonucleotides, and liposomes. They are typically highly cationic and rich in arginine and lysine amino acids. Examples of such peptides include TAT cell-penetrating peptide (GRKKRRQRRRPQ); MAP (KLALKLALKALKAALKLA); Penetratin or Antenapedia PTD (RQIKWFQNRRMKWKK); Penetratin-Arg: (RQIRIWFQNRRMRWRR); antitrypsin (358-374): (CSIPPEVKFNKPFVYLI); Temporin L: (FVQWFSKFLGRIL-NH2); Maurocalcine: GDC(acm) (LPHLKLC); pVEC (Cadherin-5): (LLIILRRRIRKQAHAHSK); Calcitonin: (LGTYTQDFNKFHTFPQTAIGVGAP); Neurturin: (GAAEAAARVYDLGLRRLRQRRRLRR ERVRA); Penetratin: (RQIKIWFQNRRMKWKKGG); TAT-HA2 Fusion Peptide: (RRRQRRK KRGGDIMGEWGNEIFGAIAGFLG); TAT (47-57) Y(GRKKRRQRRR); SynBI (RGGRLSY SRRRFSTSTGR); SynB3 (RRLSYSRRRF); PTD-4 (PIRRRKKLRRL); PTD-5 (RRQRRT SKLMKR); FHV Coat-(35-49) (RRRRNRTRRNRRRVR); BMV Gag-(7-25) (KMTRAQRR AAARRNRWTAR); HTLV-II Rex-(4-16) (TRRQRTRRARRNR); HIV-1 Tat (48-60) or D-Tat (GRKKRRQRRRPPQ); R9-Tat (GRRRRRRRRRPPQ); Transportan (GWTLNSAGYLLGKINL KALAALAKKIL chimera); SBP or Human P1 (MGLGLHLLVLAAALQGAWSQPKKKRKV); FBP (GALFLGWLGAAGSTMGAWSQPKKKRKV); MPG (ac-GALFLGFLGAAGSTMGAW SQPKKKRKV-cya (wherein cya is cysteamine)); MPG(ΔNLS) (ac-GALFLGFLGAAGSTMG AWSQPKSKRKV-cya); Pep-1 or Pep-1-Cysteamine (ac-KETWWETWWTEWSQPKKKRKV-cya); Pep-2 (ac-KETWFETWFTEWSQPKKKRKV-cya); Periodic sequences, Polyarginines (R×N (3<N<17) chimera); Polylysines (K×N (3<N<17) chimera); (RAca)6R; (RAbu)6R; (RG)6R; (RM)6R; (RT)6R; (RS)6R; R10; (RA)6R; and R7.

A “dye” refers to small organic molecules having a molecular weight (actual, not number average) of 2,000 Da or less or a protein which is able to emit light. Non-limiting examples of dyes include fluorophores, chemiluminescent or phosphorescent entities. For example, dyes useful in the present technology include but are not limited to cyanine dyes (e.g., Cy2, Cy3, Cy5, Cy5.5, Cy7, and sulfonated versions thereof), fluorescein isothiocyanate (FITC), ALEXA FLUOR® dyes (e.g., ALEXA FLUOR® 488, 546, or 633), DYLIGHT® dyes (e.g., DYLIGHT® 350, 405, 488, 550, 594, 633, 650, 680, 755, or 800) or fluorescent proteins such as GFP (Green Fluorescent Protein).

Metal chelating molecules refer to molecules that can chelate isotopes for imaging via PET or MRI, including but not limited to triazacyclononane phosphinic acids (TRAP), 1,4,7,10-tetraazacyclododecane-1,4,7,10-tetraacetic acid (DOTA), 1,4,7-triazacyclononane-N,N′,N″-triacetic acid (NOTA), diethylenetriaminepentaacetic acid (DTPA) or chelating peptides.

“Molecular weight” as used herein with respect to polymers refers to number-average molecular weights (Mₙ) and can be determined by techniques well known in the art including gel permeation chromatography (GPC). GPC analysis can be performed, for example, on a D6000M column calibrated with poly(methyl methacrylate) (PMMA) using triple detectors including a refractive index (RI) detector, a viscometer detector, and a light scattering detector, and N,N′-dimethylformamide (DMF) as the eluent. “Molecular weight” in reference to small molecules and not polymers is actual molecular weight, not number-average molecular weight.

The phrase “pharmaceutically acceptable” refers to molecular entities and compositions that are physiologically tolerable and do not typically produce untoward reactions when administered to the intended subject, particularly a human subject. In any embodiments, as used herein, the term “pharmaceutically acceptable” means approved by a regulatory agency of the federal or a state government or listed in the U S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils. Water or aqueous saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18th Edition, or other editions.

As used herein, a “subject” is a mammal, such as a cat, dog, rodent, pig, horse, cow, or primate. In any embodiments, the subject is a human.

The phrase “targeting ligand” refers to a ligand that binds to “a targeted receptor” that distinguishes the cell being targeted from other cells. The ligands may be capable of binding due to expression or preferential expression of a receptor for the ligand, accessible for ligand binding, on the target cells. Examples of such ligands include glucose, amino acids, all-trans retinoic acid, RVG peptide (YTIWMPENPRPGTPCDIFTNSRGKRASNG), N-acetylgalactosamine (GalNAc), mannitol, hyaluronic acid, RNA aptamers, DNA aptamers. Additional examples of such ligands include peptide ligands identified from library screens, monoclonal or polyclonal antibodies, Fab or scFv (i.e., a single chain variable region) fragments of antibodies and other molecules that bind specifically to a receptor preferentially expressed on the surface of targeted cells, or fragments of any of these molecules.

The phrase “a targeted receptor” refers to a receptor expressed by a cell that is capable of binding a cell targeting ligand. The receptor may be expressed on the surface of the cell. The receptor may be a transmembrane receptor. Examples of such targeted receptors include EGFR, αᵥβ₃ integrin, somatostatin receptor, folate receptor, prostate-specific membrane antigen, CD105, mannose receptor, estrogen receptor, GLUT1, LAT1, nicotinic acetylcholine receptors (nAChR), asialoglycoprotein receptor, and GM1 ganglioside.

In one aspect, the present technology provides self-assembled nanoparticles that include an amphiphilic copolymer, an RNP, and optionally ssODN. The amphiphilic copolymer is a water-soluble block copolymer that includes a poly(C₂₋₃ alkylene glycol) block and an acrylic block including a poly(acrylate), poly(methacrylate) and/or poly(acrylate/methacrylate)block. By water soluble, it is meant that the amphiphilic copolymer has a solubility of at least 1 mg/mL in pH 7.0 water at 25° C. The acrylic block comprises ester side chains bearing substituted or unsubstituted alkylamine groups. The RNP, ssODN, and the acrylic block of the amphiphilic copolymer form a core of the self-assembled nanoparticle, and the poly(C₂₋₃alkylene glycol) block of the amphiphilic copolymer forms the exterior of the self-assembled nanoparticle. The RNP is not covalently bound to the amphiphilic polymer, but in aqueous solution self-assembles with the amphiphilic polymer to provide a non-covalent nanoparticle.

The self-assembled NPs of the present technology may be generally spherical and are relatively small. In any embodiments, the self-assembled NPs have an average hydrodynamic diameter of less than 50 nm, less than 40 nm, or even less than 35 nm. Depending on the RNP and ssODN (if present) used, the average hydrodynamic diameter of the present self-assembled NPs may run, e.g., from about 25 nm to less than 35 nm. In any embodiments, the present self-assembled NPs have an average hydrodynamic diameter of about 30 nm, i.e., 30±3 nm.

In any embodiments of the present NPs, the poly(ethylene glycol) block has a number average molecular weight of about 90 Da to about 20 kDa. For example, the poly(ethylene glycol) block may have a number average molecular weight of about 90 Da, about 220 Da, about 500 Da, about 1 kDa, about 2 kDa, about 3 kDa, about 4 kDa, about 5 kDa, about 6 kDa, about 7 kDa, about 8 kDa, about 10 kDa, about 12 kDa, about 15 kDa, about 18 kDa, about 20k Da, or a range between and including any two of the foregoing values, e.g., about 1 kDa to about 15 kDa, or about 3 kDa to about 7 kDa, or about 5 kDa.

The poly(C₂₋₃ alkylene glycol) block of each individual amphiphilic copolymer may terminate with a variety of groups. For example, the poly(C₂₋₃ alkylene glycol) block may terminate with a group selected from hydroxy, an amino acid, a peptide, or substituted or unsubstituted alkyl, substituted or unsubstituted alkenyl, substituted or unsubstituted alkynyl, substituted or unsubstituted cycloalkyl, substituted or unsubstituted aryl, substituted or unsubstituted heteroalkyl, substituted or unsubstituted heteroaryl, or substituted or unsubstituted heterocyclyl. In any embodiments, the poly(C poly(C₂₋₃ alkylene glycol) block may terminate with a moiety selected from hydroxy, unsubstituted alkyl or a peptide.

In any embodiments of the self-assembled nanoparticle, the poly(C₂₋₃ alkylene glycol) block has the following structure of Formula I.

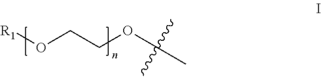

wherein
n is an integer from 2 to 500;
R¹ is H, substituted or unsubstituted alkyl, substituted or unsubstituted cycloalkyl, substituted or unsubstituted aryl, substituted or unsubstituted aralkyl, substituted or unsubstituted heteroalkyl, substituted or unsubstituted heterocyclyl, substituted or unsubstituted heterocyclylalkyl, or a moiety having a formula selected from the group consisting of

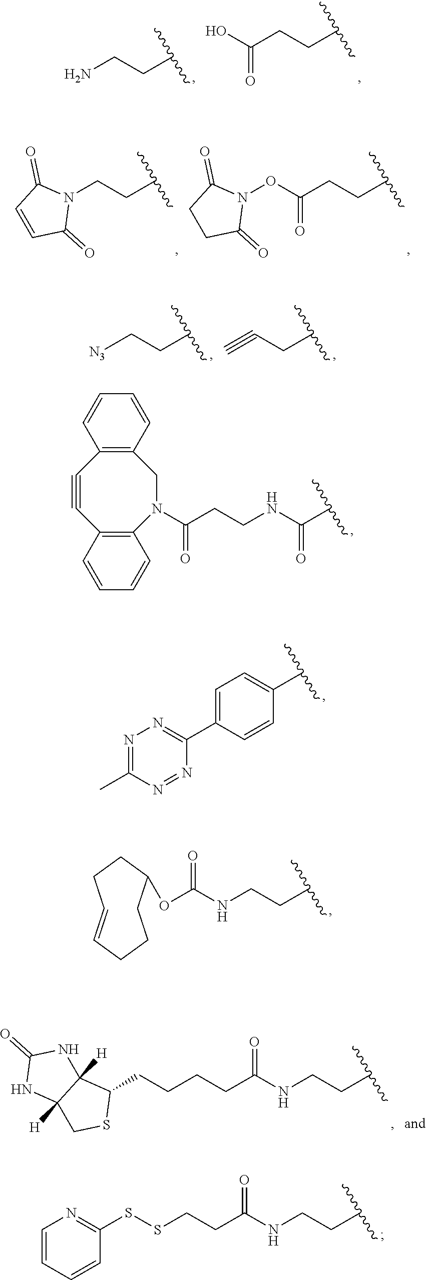

and
further wherein, for R¹ other than H, R is or is optionally substituted with a fluorophore, a metal chelating group, a targeting ligand or a cell-penetrating peptide.

In any embodiments, R¹ may be H, an unsubstituted C₁₋₄ alkyl group or a cell-penetrating peptide. In any embodiments, for R¹ other than H, R¹ is or is optionally substituted with a fluorophore, a metal chelating group, a targeting ligand, or a cell-penetrating peptide. In any embodiments, n may be 2-500, e.g., 2, 5, 10, 15, 20, 25, 50, 75, 100, 150, 200, 250, 300, 350, 400, 450, or 500 or a range between and including any two of the foregoing values such as 5-500, 10-400, 20-300, or 50-200.

The amphiphilic polymer includes an acrylic block of poly(acrylate), poly(methacrylate) or both (i.e., the poly(acrylate/methacrylate) chain may be a random copolymer or include one or more blocks of each). The acrylic block is hydrophobic relative to the poly(C₂₋₃ alkylene glycol) block and is pH-responsive. In any embodiments the acrylic block consists of poly(methacrylate). In any embodiments, the acrylic block consists of poly(acrylate). In any embodiments, the acrylic block consists of poly(acrylate/methacrylate). The acrylic block may have a number average molecular weight of about 500 Da to about 20 kDa. For example, the acrylic block may have a number average molecular weight of about 90 Da, about 220 Da, about 500 Da, about 1 kDa, about 2 kDa, about 3 kDa, about 4 kDa, about 5 kDa, about 6 kDa, about 7 kDa, about 8 kDa, about 10 kDa, about 12 kDa, about 15 kDa, about 18 kDa, about 20k Da, or a range between and including any two of the foregoing values, e.g., about 1 kDa to about 15 kDa, or about 3 kDa to about 7 kDa, or about 5 kDa.

The acrylic block (i.e., the poly(acrylate), poly(methacrylate) and/or poly(acrylate/methacrylate) includes ester side chains bearing substituted or unsubstituted alkylamine groups. In any embodiments, the ester side chains may include a C₁₋₆ alkylene group connecting the ester oxygen to the amine group, e.g., (CH₂)₁₋₆NR⁵R⁶, wherein R⁵ and R⁶ independently selected from H, substituted or unsubstituted alkyl, or substituted or unsubstituted cycloalkyl; or R⁵ and R⁶, together with the N atom to which they are attached, form a substituted or unsubstituted heterocyclyl. While not wishing to be bound by theory, it is believed that the pH-responsive nature of the amphiphilic copolymer depends on the pKa of the alkylamine side chains, which may be adjusted to a desired pKa by varying the size of the groups bound to the N atom of the alkylamine. For example, the pKa of the alkylamine side chains are higher with smaller groups such as ethyl or propyl than with longer groups such as butyl or pentyl. In any embodiments the, the amine is a cyclic amine such as a C₄₋₈ cyclic amine, optionally substituted with 1-3 additional alkyl groups, such as C₁₋₃ alkyl. In any embodiments, the amine is a C₄₋₅ cyclic amine, optionally substituted with 1, 2, or 3 methyl groups.

In any embodiments, the acrylic block comprises a methacrylate repeating subunit with the structure of Formula II:

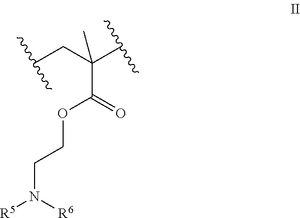

wherein:
R⁵ and R⁶ independently selected from H, substituted or unsubstituted alkyl, or substituted or unsubstituted cycloalkyl; or R⁵ and R⁶, together with the N atom to which they are attached, form a substituted or unsubstituted heterocyclyl.

In any embodiments, R⁵ and R⁶, together with the N atom to which they are attached, form a C₄₋₅ cyclic amine, optionally substituted with 1, 2, or 3 methyl groups.

In any embodiments, the acrylic block may include 2-200 of the repeating subunit of Formula II. Thus, the acrylic block may include 2, 5, 10, 15, 20, 30, 4, 50, 75, 100, 125, 150, or 200 repeating subunits of Formula II, or a range between and including any two of the foregoing values, e.g., 5-200 or 10-150.

The acrylic block and the poly(ethylene glycol) block may be joined by any suitable linkage known in the art. In any embodiments, the acrylic block and the poly(ethylene glycol) block are joined by an ester linkage.

In any embodiments, the amphiphilic copolymer may have the structure of Formula III:

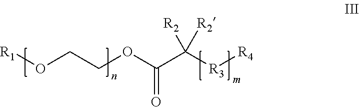

wherein
n is an integer from 2 to 500;
m is an integer from 5-200;
R¹ is H, substituted or unsubstituted alkyl, substituted or unsubstituted cycloalkyl, substituted or unsubstituted aryl, substituted or unsubstituted aralkyl, substituted or unsubstituted heteroalkyl, substituted or unsubstituted heterocyclyl, substituted or unsubstituted heterocyclylalkyl, or a moiety having a formula selected from the group consisting of

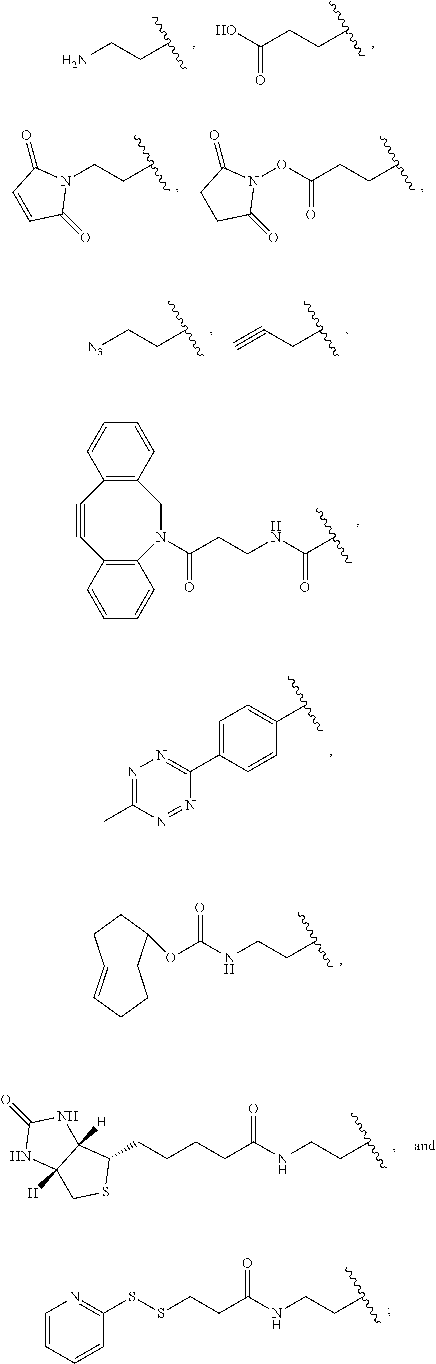

and
further wherein, for R¹ other than H, R is or is optionally substituted with a fluorophore, a metal chelating group, a targeting ligand or a cell-penetrating peptide;
R² and R^{2′} are independently selected from the group consisting of H, substituted or unsubstituted alkyl, and substituted or unsubstituted cycloalkyl;
R³ has the structure of formula II.

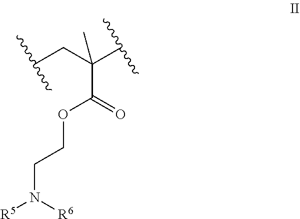

wherein:
R⁵ and R⁶ are independently selected from H, substituted or unsubstituted alkyl, or substituted or unsubstituted cycloalkyl, or R⁵ and R⁶, together with the N atom to which they are attached, form a substituted or unsubstituted heterocyclyl; and
R⁴ is H, OH, halo, or substituted or unsubstituted alkyl.

In any embodiments including the amphiphilic copolymer of Formula III, n may be 2-500, e.g., 2, 5, 10, 15, 20, 25, 50, 75, 100, 150, 200, 250, 300, 350, 400, 450, or 500 or a range between and including any two of the foregoing values such as 5-500, 10-400, 20-300, or 50-200. In any embodiments, m may be 5-200, e.g., 5, 10, 15, 20, 30, 4, 50, 75, 100, 125, 150, or 200 or a range between and including any two of the foregoing values, e.g., 5-200 or 10-150.

In any embodiments including the amphiphilic copolymer of Formula III, R¹ may be H, or unsubstituted C₁₋₆ alkyl. In any embodiments, R¹ may be or a moiety having a formula selected from the group consisting of

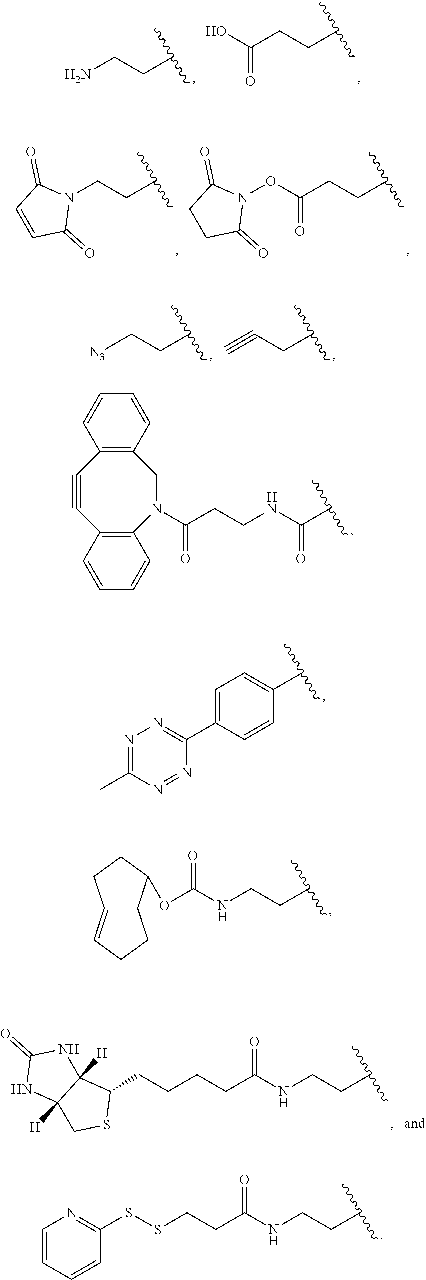

In any embodiments, R¹ is not H, but R¹ is or is optionally substituted with a fluorophore, a metal chelating group, a targeting ligand or a cell-penetrating peptide. The fluorophore, a metal chelating group, a targeting ligand or a cell-penetrating peptide may but need not be selected from any of those disclosed herein.

In any embodiments including the amphiphilic copolymer of Formula III, R⁵ and R⁶ are independently selected from unsubstituted alkyl or R⁵ and R⁶, together with the N atom to which they are attached, form a C₄₋₈ heterocyclyl, optionally substituted with 1, 2, or 3 C₁₋₃ alkyl groups. In any embodiments, R⁵ and R⁶ are independently ethyl, n-propyl, n-butyl, n-pentyl, or R⁵ and R⁶, together with the N atom to which they are attached, form a C₅₋₆ heterocyclyl, optionally substituted with 1 or two methyl groups. In any embodiments, R⁵ and R⁶, together with the N atom to which they are attached, form a C₆ heterocyclyl, e.g., azepane.

The present NPs optionally include a ssODN. In any embodiments, the NP includes an ssODN. For example, the RNP may be selected from the group consisting of Cas9, Cas12, and Cas13. In any embodiments, the RNP is Cas9 and Cas9 comprises Cas9 nuclease and RNA selected from sgRNA or the combination of crRNA and tracrRNA. In any embodiments, the RNP is Cas12 and Cas12 comprises Cas12 nuclease and crRNA. In any embodiments, the RNP is Cas13 and Cas13 comprises Cas13 nuclease and RNA crRNA. In any embodiments, the RNP is Cas9 and the RNA is sgRNA having the sequence:

5′-NNNNNNNNNNNNNNNNNNNNGUUUUAGAGCUAGAAAUAGCAAGUUAA
AAUAAGGCUAGUCCGUUAUCAACUUGAAAAAGUGGCACCGAGUCGGUGCU
UUU-3′;
5′-NNNNNNNNNNNNNNNNNNNNGUUUAAGAGCUAUGCUGCGAAUACGAG
AUGCGGCCGCCGACCAGAAUCAUGCAAGUGCGUAAGAUAGUCGCGGGUCG
GCGGCUCGUAUUCGCAGCAUAGCAAGUUUAAAUAAGGCUAGUCCGUUAUC
AACUUGAAAAAGUGGCACCGAGUCGGUGCUUUU-3′;
5′-NNNNNNNNNNNNNNNNNNNNGUUUAAGAGCUAUGCUGGAAACAGCAU
AGCAAGUUUAAAAAGGCUAGUCCGUUAUCAACUUCGAAUACGAGAUGCGG
CCGCCGACCAGAAUCAUGCAAGUGCGUAAGAUAGUCGCGGGUCGGCGGCU
CGUAUUCGGAAAAAGUGGCACCGAGUCGGUGCUUUU-3′;
5′-NNNNNNNNNNNNNNNNNNNNGUUUAAGAGCUAUGCUGGAAACAGCAU
AGCAAGUUUAAAUAAGGCUAGUCCGUUAUCAACUUGAAAAAGUGGCACCG
AGUCGGUGCCGAAUACGAGAUGCGGCCGCCGACCAGAAUCAUGCAAGUGC
GUAAGAUAGUCGCGGGUCGGCGGCUCGUAUUCGUUUU-3′ .

In any embodiments, the RNP is Cas9 and the RNA is the combination of crRNA and tracrRNA having the sequence:

5′-NNNNNNNNNNNNNNNNNNNNGUUUUAGAGCUAUGCUGUUUUG-3′;
5′-UAGCAAGUUAAAAUAAGGCUAGUCCGUUAUCAACUUGAAAAAGUGGC
ACCGAGUCGGUGCUUUU-3′;
5′-GGAACCAUUCAAAACAGCAUAGCAAGUUAAAAUAAGGCUAGUCCGUU
AUCAACUUGAAAAAGUGCACCGAGUCGGUGCUUUU-3′ .

In any embodiments, the RNP is Cas12, or Cas13, and the RNA is crRNA and/or tracrRN A having the sequence:

5′-UAAUUUCUACUCUUGUAGAUNNNNNNNNNNNNNNNNNNNNN-3′;
5′-GGUAAUUUCUACUAAGUGUAGAUNNNNNNNNNNNNNNNNNNNNNNN-
3′;
5′-GTCGGATCACTGAGCGAGCGATCTGAGAAGTGGCACNNNNNNNNNNN
NNNNNNNNN-3′;
5′-GTCTAAAGGACAGAATTTTCAACGGGTGTGCCAATGGCCACTTTCCA
GGTGGCAAAGCCCGTTGAACTTCTCAAAAAGAACGCTCAGTGTTCTGAC-
3′;
5′-GAUUUAGACUACCCCAAAAACGAAGGGGACUAAAACNNNNNNNNNNN
NNNNNNNNNNNNNNNNN-3′;
5′-NNNNNNNNNNNNNNNNNNNNNNNNNNNNNNGUUGUGGAAGGUCCAGU
UUUGGGGGCUAUUACAACA-3′;
5′-AACCCCUACCAACUGGUCGGGGUUUGAAACNNNNNNNNNNNNNNNNN
NNNNNN-3′.

In any embodiments, the SSODN may be 20-200 nucleotides in length. For example, the SSODN may be 20, 40, 60 80, 100, 120, 140, 160, 180, 200, or a range between and including any two of the foregoing values, such as 40-200 or 40-140 or 60-120.

In any embodiments, the weight ratio of protein/nucleic acid complex to amphiphilic copolymer is 1:1 to 1:100. For example, the weight ratio may be 1:1, 1:2, 1:5, 1:10, 1:20, 1:30, 1:40, 1:50, 1:60, 1:70, 1:80, 1:90, 1:100 or a range between and including any two of the foregoing values.

In another aspect, the present technology provides a pharmaceutical composition including any of the self-assembled nanoparticles described herein and a pharmaceutically acceptable aqueous carrier. The pharmaceutically acceptable aqueous carrier may have a pH of 5-9. Thus, the pH may be 5, 5.5, 6, 6.5, 7, 7.5, 8, 8.5, 9 or a range between and including any two of the foregoing values such as 6.5-8 or 7-8. Many pharmaceutically acceptable aqueous carriers may be used, e.g., the carrier may be saline and or may include one or more buffers selected the group consisting of tris(hydroxymethyl)aminomethane (Tris), phosphate-buffered saline (PBS), bicarbonate, 2-(N-morpholino)ethanesulfonic acid (MES), 3-morpholinopropane-1-sulfonic acid (MOPS), and 4-(2-hydroxyethyl)-1-piperazineethanesulfonic acid (HEPES).

In another aspect, the present technology provides methods of editing a nucleic acid, e.g., a targeted gene in a subject, the method comprising administering an effective amount of any nanoparticle as described herein or a pharmaceutical composition thereof to the subject, whereby the RNP is targeted to the gene to be edited and edits the targeted gene. In any embodiments, the targeted gene is a reporter gene. In any embodiments, the targeted gene is a therapeutic target gene that is desirable to inactivate or edit to provide a therapeutic benefit to the subject. Thus, non-limiting examples of the targeted gene include BFP, GFP, mCherry, SV40 PolyA, DMD, CFTR, PCSK9, PTEN, SIRPA, KRAS, APP, NLRP3, or BRAF.

The compositions described herein can be formulated for various routes of administration, for example, by parenteral, intravitreal, intrathecal, intracerebroventricular, rectal, nasal, vaginal administration, direct injection into the target organ, or via implanted reservoir. Parenteral or systemic administration includes, but is not limited to, subcutaneous, intravenous, intraperitoneal, and intramuscular injections. The following dosage forms are given by way of example and should not be construed as limiting the instant present technology.

Injectable dosage forms generally include solutions or aqueous suspensions which may be prepared using a suitable dispersant or wetting agent and a suspending agent so long as such agents do not interfere with formation of the self-assembled NPs described herein. Injectable forms may be prepared with acceptable solvents or vehicles including, but not limited to sterilized water, phosphate buffer solution, Ringer's solution, 5% dextrose, or an isotonic aqueous saline solution.

Besides those representative dosage forms described above, pharmaceutically acceptable excipients and carriers are generally known to those skilled in the art and are thus included in the instant present technology. Such excipients and carriers are described, for example, in “Remingtons Pharmaceutical Sciences” Mack Pub. Co., New Jersey (1991), which is incorporated herein by reference. Exemplary carriers and excipients may include but are not limited to USP sterile water, saline, buffers (e.g., phosphate, bicarbonate, etc.), tonicity agents (e.g., glycerol), and the like.

Specific dosages may be adjusted depending on conditions of disease, the age, body weight, general health conditions, sex, and diet of the subject, dose intervals, administration routes, excretion rate, and combinations of drug conjugates. Any of the above dosage forms containing effective amounts are well within the bounds of routine experimentation and therefore, well within the scope of the instant present technology. By way of example only, such dosages may be used to administer effective amounts of the present NPs to the patient and may include 0.1, 0.2, 0.3, 0.4, 0.5, 0.75, 1.0, 1.5, 2.0, 2.5, 3.0, 4.0, 5.0, 6.0, 7.0, 8.0, 9.0, 10, 11, 12, 13, 14, 15 mg/kg or a range between and including any two of the foregoing values such as 0.1 to 15 mg/kg. Such amounts may be administered parenterally as described herein and may take place over a period of time including but not limited to 5 minutes, 10 minutes, 20 minutes, 30 minutes, 45 minutes, 1 hour, 2 hours, 3 hours, 5 hours, 10 hours, 12, hours, 15 hours, 20 hours, 24 hours or a range between and including any of the foregoing values. The frequency of administration may vary, for example, once per day, per 2 days, per 3 days, per week, per 10 days, per 2 weeks, or a range between and including any of the foregoing frequencies. Alternatively, the compositions may be administered once per day on 2, 3, 4, 5, 6 or 7 consecutive days. A complete regimen may thus be completed in only a few days or over the course of 1, 2, 3, 4 or more weeks.

In another aspect, the present technology provides kits including the components needed to prepare any of the compositions described herein. For example, a kit may include a package containing an amphiphilic copolymer as described herein, an RNP as described herein, and optionally an ssODN. The kit may also include other reagents needed for preparing the NPs of the present technology as well as directions for preparing the NPs. Alternatively, the user may provide the RNP, ssODN or both. The present kits allow the user to prepare the delivery composition described herein by simply mixing the amphiphilic polymer and RNP (with or without an ssODN) in aqueous solution (e.g., any suitable buffer), waiting for a short period of time (e.g., 15 minutes to an hour, preferably about a half hour, and then using them (e.g., adding to a cell culture or injecting into a subject). The kit may supply a mixture of copolymers that differ in the their structure, such as by bearing acrylic or poly(alkylene glycol) blocks of different number average molecular weight and/or by having differing terminal groups on the poly(alkylene glycol) blocks (e.g., CPP, alkyl group, metal chelating group, etc.), or the user may attach targeting ligands, imaging ligands, and/or cell-penetrating peptides to a suitable copolymer (e.g., one with a terminal maleimide group) to further customize the NP.

The examples herein are provided to illustrate advantages of the present technology and to further assist a person of ordinary skill in the art with preparing or using the NPs compositions of the present technology. To the extent that the compositions include ionizable components, salts such as pharmaceutically acceptable salts of such components may also be used. The examples herein are also presented in order to more fully illustrate the preferred aspects of the present technology. The examples should in no way be construed as limiting the scope of the present technology, as defined by the appended claims. The examples can include or incorporate any of the variations or aspects of the present technology described above. The variations or aspects described above may also further each include or incorporate the variations of any or all other variations or aspects of the present technology.

### EXAMPLES

### General

Materials. Tetrahydrofuran (THF), hexane, ethyl acetate, chloroform, anhydrous dimethylformamide (DMF), tris(2-carboxyethyl)phosphine hydrochloride (TCEP), tris(hydroxymethyl)aminomethane hydrochloride solution (Tris HCl, 1M), Y-27632 dihydrochloride and bovine serum albumin (BSA) were purchased from Fisher Scientific. Triethylamine, NaOH, NaCl, hydrochloric acid (HCl), methacryloyl chloride, diethyl ether, CuBr, and anhydrous MgSO₄ were obtained from Sigma-Aldrich. N,N,N′,N″,N″-pentamethyldiethylenetriamine (PMDETA) and 2-(azepan-1-yl)ethanol was purchased from Tokyo Chemical Industry Co., Ltd. and ChemScene, LLC, respectively. Methoxy-poly(ethylene glycol)-hydroxy (mPEG-OH, Mw-5 kDa) and maleimide-poly(ethylene glycol)-hydroxy (Mal-PEG-OH, Mw-5 kDa) were provided by JenKem Technology USA Inc. Cell-penetrating peptide Cys-TAT (47-57) (CPP, sequence: CYGRKKRRQRRR-NH₂) was obtained from GenScript, Inc. Ultrapure water was freshly produced using Milli-Q system (MilliporeSigma).

Nanoparticle characterization. The hydrodynamic diameters and zeta potentials of NHEJ-NPs and HDR-NPs were measured by dynamic light scattering (DLS, ZetaSizer Nano ZS90, Malvern Panalytical Ltd) and analyzed using Zetasizer software v7.01 (Malvern Panalytical Ltd). The morphology of NHEJ-NPs and HDR-NPs was studied by transmission electron microscopy (FEI Tecnai 12, 120 kV, Fischione Instruments, Inc.) and analyzed by ImageJ 1.51j8 (NIH).

Gel retardation assay. Gels were cast using 2% (w/v) agarose solution in tris-acetate-ethylenediaminetetraacetic acid (TAE) buffer solution containing SYBR Safe DNA Gel Stain (Thermo Fisher Scientific). Free Cas9 RNP, sgRNA, ssODN, Cas9 RNP+ssODN, NHEJ-NP, and HDR-NP with an equivalent dosage of 0.5 μg nucleic acids per well were loaded in the gel, respectively. Electrophoresis was conducted with a voltage at 110 V for 30 min using Mini-Sub Cell GT Horizontal Electrophoresis System (Bio-Rad Laboratories, Inc.). Retardation of all tested samples was visualized by a UV Illuminator (Bio-Rad Laboratories, Inc.) and analyzed by Image Lab software (Bio-Rad).

Cell culture. Human embryonic kidney (HEK293) cells, GFP-expressing HEK293 cells, and BFP-expressing HEK293 cells were cultured at 37° C. in a 5% CO₂ atmosphere with the Dulbecco's Modified Eagle Medium (Thermo Fisher Scientific) supplemented with 10% (v/v) fetal bovine serum (Thermo Fisher Scientific) and 1% (v/v) Penicillin-Streptomycin (Thermo Fisher Scientific). BFP-expressing WA09 human embryonic stem cells (hESCs) were maintained at 37° C. in a 5% CO₂ atmosphere in the StemFlex™ Medium (Thermo Fisher Scientific) on Matrigel (Corning) coated tissue culture polystyrene plates (BD Falcon). Cells were passaged every 3-4 days at a 1:10 ratio using Versene solution (Thermo Fisher Scientific). ROCK inhibitor Y-27632 (10 μM) was periodically supplemented to the culture media to enhance the viability of stem cells.

Assaying gene editing efficiency of NHEJ-NPs and HDR-NPs. GFP-expressing or BFP-expressing HEK293 cells were seeded in 96-well plates (5,000 cells per well). BFP-expressing hESCs were seeded in 96-well plates coated with Matrigel (5,000 cells per well). After 24 h, various NHEJ-NP or HDR-NP formulations were prepared freshly and added to the cells with dosages of RNP and ssODN (for HDR-NP only) both at 3.13 pmol per well. Lipofectamine 2000 (Thermo Fisher Scientific) or Lipofectamine CRISPRMAX (Thermo Fisher Scientific) formulations complexed with the same dosage of RNP or RNP+ssODN were prepared according to the manufacturer's manual. After 96 h, cells were detached by Trypsin-EDTA (0.25%, Thermo Fisher Scientific, for HEK293 cells) or Versene solution (for hESCs) and collected for flow cytometry analysis (Attune N×T, Thermo Fisher Scientific). The percentage of GFP-negative cells was used for NHEJ efficiency assay in GFP-expressing HEK293 cells. The percentage of GFP-positive cells was used for HDR efficiency assay in BFP-expressing HEK293 cells and BFP-expressing hESCs. Data were analyzed by FlowJo.

Cell viability. HEK293 cells were seeded in 96-well plates (10,000 cells per well). After 24 h, cells were treated with Lipofectamine 2000, Lipofectamine CRISPRMAX, NHEJ-NP, and HDR-NP all at 6.25 pmol, respectively. After 48 h of incubation, a standard cell counting kit-8 assay (CCK-8, Dojindo Molecular Technologies, Inc.) was performed according to the manufacturer's instructions. Each well was added with 10 μl CCK-8 solution and incubated at 37° C. for 4 h. Thereafter, the absorbance at 450 nm and 650 nm (as a reference) of each well was measured using a microplate reader (Promega Corporation). The cell viability relative to the untreated cells was finally calculated.

Cellular uptake study. To prepare fluorophore-labeled NHEJ-NP and CPP-NHEJ-NP, negative control crRNA and Atto 550-labeled tracrRNA (Integrated DNA Technologies, Inc.) were first complexed at a 1/1 molar ratio for 10 min at 20° C. to form gRNA and then complexed with Cas9 nuclease at 1/1 molar ratio to form Atto 550-labeled Cas9 RNP. Thereafter, Atto 550-labeled NHEJ-NP and CPP-NHEJ-NP were prepared following the same protocol mentioned above. HEK293 cells were seeded in 96-well plates (10,000 cells per well) 24 h before treatments. For endocytosis pathway study, a variety of inhibitors including wortmannin (10 μg/mL), chlorpromazine (10 μg/mL), genistein (100 μg/mL), and methyl-β-cyclodextrin (5 mM) dissolved in serum-containing cell culture media were added to HEK293 cells, respectively. The cells were then incubated at 37° C. for 30 min and washed thoroughly with 1×DPBS three times. Atto 550-labeled NHEJ-NP or CPP-NHEJ-NP was then added to the cells with a dosage of RNP at 6.25 pmol per well. After 4 h incubation at 37° C., the media were aspirated, and the cells were washed with 1×DPBS three times and collected after trypsinization. For incubation at 4° C., cells were pretreated at 4° C. for 30 min. Atto 550-labeled NHEJ-NP or CPP-NHEJ-NP was then added to the cells (6.25 pmol RNP per well), which were incubated at 4° C. for 4 h before media removal. The cells were washed with 1×DPBS three times and collected after trypsinization. The cellular uptake of NHEJ-NP and CPP-NHEJ-NP in HEK293 cells was assayed by flow cytometry to quantify Atto 550-positive cells.

Intracellular trafficking. HEK293 cells were seeded in an 8-well chamber slide (20,000 cells per well) and cultured for 24 h before use. Atto 550-labeled NHEJ-NP was formulated as mentioned above and added to the cells at 6.25 pmol RNP per well. At certain time points (i.e., 0.5 h, 2 h, and 4 h), the cells were washed with 1×DPBS three times and treated with LysoTracker Green DND-26 (75 nM, Thermo Fisher Scientific) and Hoechst 33342 (1 g/mL, Thermo Fisher Scientific) to stain endosomes/lysosomes and nuclei, respectively. Cells were washed with 1×DPBS three times again and then imaged using an AR1 confocal microscope (Nikon). Images were exported using NIS Viewer v5.21 (Nikon).

Animals. All animal studies strictly conformed to the Guide for the Care and Use of Laboratory Animals (National Institutes of Health) and protocols approved by the Institutional Animal Care and Use Committee (IACUC) at the University of Wisconsin-Madison. B6.Cg-Gt(ROSA)26Sor^{tm14(cAG-tdTomato)Hze}/J mice (Ai14 mice, 4-8 weeks old, male and female, JAX stock #007914) were randomly divided into groups and used to assay the in vivo gene editing efficiency of NHEJ-NP. C57BL/10ScSn-Dmd^{mdx}/J (mdx mice, 4 weeks old, male, JAX stock #001801) and C57BL/6J (wild-type mice, 4 weeks old, male, JAX stock #000664) were randomly divided into groups and used to assay the in vivo gene editing efficiency of HDR-NP for DMD treatments. All mice were maintained under a tightly controlled temperature (22° C.), humidity (40-50%), light/dark (12/12 h) cycle conditions, with water and food ad libitum. The mdx mice were specially fed with a diet containing 6% fat (LabDiet® 5K52). Before injections, the mice were anesthetized with 3% isoflurane for 8-10 min followed by 1% isoflurane during the injection.

In vivo gene editing in Ai14 mice. Cas9 nuclease was complexed with synthetic sgRNA (Integrated DNA Technologies, Inc.) designed for the excision of SV40 polyA blocks to form Cas9 RNP. NHEJ-NP was then prepared as described above and used for intravenous and intramuscular injections. For intravenous injections, two groups of Ai14 mice (n=3 per group, ˜20 μg in body weight) were injected with 100 μl of PBS (negative control) and NHEJ-NP solutions (containing 1.2 μg/l Cas9 RNP, equivalent to 6 mg/kg) via retro-orbital injections. For intramuscular injections, mice were administered with 1×DPBS (negative control) or NHEJ-NP solutions (containing 1.2 μg/l Cas9 RNP) in the tibialis anterior muscle (40 μl, n=3 muscles per group) using 32-gauge syringes. After 7 days, the whole blood of mice was collected from the sublingual vessel into heparin-coated collection tubes and the serum was separated by centrifugation at 1,500×g for 10 min for hematological analysis. Thereafter, mice were euthanized and their major organs and tissues including heart, liver, spleen, lung, kidney, and muscles were stored immediately in 4% neutral-buffered formalin at 4° C. (for fixation and cryosection), or flash-frozen by liquid nitrogen and stored at −80° C. (for other studies). For intratracheal injections, three groups of Ai14 mice (n=3 per group, ˜20 μg in body weight) were injected with 50 μl of PBS (negative control), isotonic NHEJ-NP, or hypotonic NHEJ-NP solutions (containing 1.2 μg/l Cas9 RNP, equivalent to 3 mg/kg), respectively. The anesthetized mouse was fixed on an intubating platform with its mouth open and illuminated by an LED light. Meanwhile, the mouse tongue was controlled using curved blunt-ended forceps. A PE-10 tubing attached to the 1-mL syringe containing the PBS or NHEJ-NP solution was inserted 0.5-1.0 cm into the trachea and then the solution was instilled. After instillation, the PE-10 tubing was kept in place for 5 seconds. The mouse was maintained in the same position on the intubating platform for another 30 seconds and finally placed on a heating pad for recovery. After 14 days, mice were euthanized, and their lungs and other organs including heart, liver, spleen, kidney were collected. The organs were stored immediately in 4% neutral-buffered formalin at 4° C. or flash-frozen by liquid nitrogen and stored at −80° C.

In vivo imaging assay. TdTomato expression in the organs and tissues of the mice was first assayed by in vivo imaging system (IVIS) equipped with an excitation wavelength at 554 nm and an emission wavelength at 581 nm. Thereafter, small portions of the livers were collected, treated with lysis buffer, and sonicated for tissue homogenization. The fluorescence intensity of homogenized samples was then quantified again by IVIS.

In vivo gene editing in mdx mice. Cas9 nuclease was complexed with synthetic sgRNA (Integrated DNA Technologies, Inc.) targeting the exon 23 in the Dmd gene and ssODN for gene correction. HDR-NP was then prepared as described above. For intramuscular injections, mice were administered with 1×DPBS (negative control, n=7 animals) or HDR-NP solutions (containing 1.2 μg/l Cas9 RNP and 0.36 μg/l ssODN (Cas9 RNP/ssODN=1/1, mol/mol), n=11 animals) in both tibialis anterior muscles (20 μl), both gastroenemius muscles (20 μl) and both triceps brachii muscles (40 μl) using 32-gauge syringes (total 6 muscles per mouse). Wild-type mice (n=6 animals) were used as the positive control without injections. The hanging times for these mice were tested weekly. After 28 days, mice were euthanized and their major organs including heart, liver, spleen, lung, and kidney, and muscles (including tibialis anterior, gastrocnemius, and triceps brachii muscles) were collected and stored immediately in 4% neutral-buffered formalin at 4° C. (for fixation and cryosection), or flash-frozen by liquid nitrogen and stored at −80° C. (for other studies). For the gene sequencing study, mdx mice were intramuscularly injected with 40 μl of 1×DPBS (negative control) or HDR-NP solutions (containing 1.2 μg/l Cas9 RNP and 0.36 μg/l ssODN) in tibialis anterior muscle. After 7 days, mice were euthanized, and the tibialis anterior muscles were collected for genomic DNA extraction.

Hanging time assay. Four-limb hanging time assays were performed on untreated wild-type mice and mdx mice with or without HDR-NP treatments weekly after the treatments, following the previously reported method^{8,36}. In brief, the apparatus was set up by positioning a metal grid 25 cm above a big cage for rats. Soft bedding was placed at the cage bottom to prevent mice from harming themselves when falling. Thereafter, a mouse was placed on the grid and the grid was inverted above the cage, so the mouse started to hang. The test session ended if the mouse was able to hang for 600 seconds. Each mouse was tested three times to determine the representative hanging time.

Sanger Sequencing. The genomic DNA was isolated from muscles using Monarch® Genomic DNA Purification Kit (New England Biolabs, #T3010) following the manufacturer's protocol. Genomic PCR was performed using Q5@High-Fidelity 2× Master Mix (New England Biolabs, #M0492) with genomic DNA templates (50 ng) and customized primers (0.5 μM, Integrated DNA Technologies, Inc.), following the manufacturer's protocol and thermocycling conditions: 30 cycles of 98° C. for 10 s, 67° C. for 30 s, and 72° C. for 30 s with a final extension time at 72° C. for 2 min. PCR products were purified using Monarch® PCR & DNA Cleanup Kit (New England Biolabs, #T1030) and quantified by Nanodrop One Microvolume UV-Vis Spectrophotometer (Thermo Fisher Scientific). Sanger sequencing was finally performed with a sample containing 20 ng purified PCR product and 25 pmol primers by GENEWIZ. The sequencing results were analyzed using ApE software v3.0.5 (https://jorgensen.biology.utah.edu/wayned/ape/), SnapGene Viewer v5.3, and TIDE web tool (http://shinyapps.datacurators.nl/tide/)⁸.

Immunofluorescence staining. TdTomato expression was further investigated via immunofluorescence staining. The collected tissues and organs were embedded in optimal cutting temperature (OCT) compound blocks (Thermo Fisher Scientific), and the blocks were sectioned into 8 μm slices. For samples from Ai14 mice, sections were stained with anti-RFP primary antibody (Abcam, ab152123, 1:1,000) for TdTomato. Hepatocytes were stained by HepPar1 (NBP2-45272, diluted at 1:100, Novus Biologicals). Endothelial cells were stained by anti-CD31 antibody (Abcam, ab56299, 1:400). Macrophages/Kupffer cells were stained by anti-F4/80 antibody [CI:A3-1] (Abcam, ab6640, 1:100). Epithelial cells were stained by anti-Ep-CAM antibody (Santa Cruz Biotechnology, sc-53532, 1:200). Pulmonary alveolar type I (AT1) cells were stained by Anti-Hop Antibody (E-1) (Santa Cruz Biotechnology, sc-398703, 1:200). For samples from the DMD mice, the sections were stained with dystrophin antibody (Santa Cruz Biotechnology, sc-47760, 1:100). All sections were further stained by secondary antibodies (Abcam, ab150113, ab150080, ab150155, all 1:1,000) and DAPI (Abcam, ab228549, 1:1,000) for visualization. After mounting on glass slides, the sections were imaged using a Nikon AR1 confocal microscope (for Ai14 mouse samples) or Zeiss LSM 710 confocal microscope (for mdx mouse and wild-type mouse samples). Images were exported using NIS Viewer v5.21 (Nikon) or ZEN blue v3.3 (Zeiss).

Trichrome staining. Trichrome staining of muscle sections was performed using Masson's Trichrome Stain Kit (G-Biosciences, BAQ085) following the manufacturer's protocol. The stained sections were observed under an optical microscope.

In vivo biocompatibility assay. Hematological analysis was performed using the collected mouse serum to evaluate the key elements of the blood biochemical profile using VetScan Preventive Care Profile Plus rotors (Abaxis) in a VetScan VS2 blood chemistry analyzer (Abaxis), following the manufacturer's protocol. Moreover, to evaluate the systemic or local toxicity, dehydrated and fixed organs (i.e., heart, liver, spleen, lung, kidney) and tibialis anterior muscles were embedded in OCT and the blocks were sectioned. The sections were stained with hematoxylin and eosin (H&E) and observed under an optical microscope.

In vivo immunogenicity assay. The immunogenicity of HDR-NPs in treated muscles was analyzed by RT-PCR. In brief, the muscle samples collected from mdx mice were stored in RNAlater solution (Thermo Fisher Scientific) at −20° C. until RNA extraction. RNA in these samples was extracted using TRIzol reagent (Thermo Fisher Scientific) following manufacturer's protocol and quantified by Nanodrop One Microvolume UV-Vis Spectrophotometer. cDNA was then synthesized using iScript™ Reverse Transcription Supermix for RT-PCR (Bio-Rad Laboratories, Inc.) following the manufacturer's protocol. Quantitative RT-PCR was finally performed using iTaq Universal SYBR Green Supermix (Bio-Rad Laboratories, Inc.) with cDNA templates (10 ng) and customized primers (0.5 μM, Integrated DNA Technologies, Inc.) for genes of interest on a CFX96 Touch Real-Time PCR Detection System (Bio-Rad Laboratories, Inc.). The thermocycling conditions used for PCR were 40 cycles of 95° C. for 5 s and 60° C. for 30 s. Melt curve analysis was performed at the end of PCR experiments. Glyceraldehyde 3-phosphate dehydrogenase (GAPDH) was selected as the reference gene for data analysis. Data were analyzed using CFX Maestro 2.0 (Bio-Rad). Sequences of primers used in this work are summarized in Table 1.

TABLE 1
Sequences of primers for PCR and RT-PCR.

| Gene | Sequence (5′ to 3′) |
|---|---|
| Dmd | GCGTGTTAGTGTAAATGAACTTCTA |
| CCACCAACTGGGAGGAAAG | |
| GAPDH | TGAGGCCGGTGCTGAGTATGTCG |
| CCACAGTCTTCTGGGTGGCAGTG | |
| Eotaxin | GCTACAGGAGAATCACCAGTGG |
| GGAATCCTGCACCCACTTCTTC | |
| IFN-γ | CAGCAACAGCAAGGCGAAAAAGG |
| TTTCCGCTTCCTGAGGCTGGAT | |
| IL-1α | CGAAGACTACAGTTCTGCCATT |
| GACGTTTCAGAGGTTCTCAGAG | |
| IL-1β | TGGACCTTCCAGGATGAGGACA |
| GTTCATCTCGGAGCCTGTAGTG | |
| IL-6 | CTGCAAGAGACTTCCATCCAG |
| AGTGGTATAGACAGGTCTGTTGG | |
| IL-9 | TCCACCGTCAAAATGCAGCTGC |
| CCGATGGAAAACAGGCAAGAGTC | |
| IL-10 | CGGGAAGACAATAACTGCACCC |
| CGGTTAGCAGTATGTTGTCCAGC | |
| IL12-p35 | ACGAGAGTTGCCTGGCTACTAG |
| CCTCATAGATGCTACCAAGGCAC | |
| IL12-p40 | TTGAACTGGCGTTGGAAGCACG |
| CCACCTGTGAGTTCTTCAAAGGC | |
| IL-13 | AACGGCAGCATGGTATGGAGTG |
| TGGGTCCTGTAGATGGCATTGC | |
| IL-17A | GAAGCTCAGTGCCGCCA |
| TTCATGTGGTGGTCCAGCTTT | |
| CXCL1 | TCCAGAGCTTGAAGGTGTTGCC |
| AACCAAGGGAGCTTCAGGGTCA | |
| CCL2 | GCTACAAGAGGATCACCAGCAG |
| GTCTGGACCCATTCCTTCTTGG | |
| CCL3 | ACTGCCTGCTGCTTCTCCTACA |
| ATGACACCTGGCTGGGAGCAAA | |
| CCL4 | ACCCTCCCACTTCCTGCTGTTT |
| CTGTCTGCCTCTTTTGGTCAGG | |
| CCL5 | CCTGCTGCTTTGCCTACCTCTC |
| ACACACTTGGCGGTTCCTTCGA | |
| TNF-α | GGTGCCTATGTCTCAGCCTCTT |
| GCCATAGAACTGATGAGAGGGAG | |
| NF-κB | CTGGCAGCTCTTCTCAAAGC |
| TCCAGGTCATAGAGAGGCTCA | |

Intranasal administration in Ai14 mice. Two groups of Ai14 mice (˜20 μg in body weight, n=3 per group) were injected with 100 μl of PBS (negative control) or solution of NHEJ-NP encapsulating Cas9 RNP (containing 1.2 μg/l Cas9 RNP, equivalent to 6 mg/kg). In brief, PBS or NHEJ-NP solution was first loaded in an insulin syringe with a 31-gauge needle. While the anesthetized mouse was fixed in an upright position, the needle tip was placed near the mouse's left nostril. The solution was then slowly injected as small droplets (˜3 μl in volume). After all the solution was injected, the mouse was held in the upright position for 15 seconds, and then put back to the cage. After 14 days, mice were euthanized, and their lungs were collected.

Statistical analysis. Data for each experiment were analyzed without further pre-processing. Results are presented as mean±standard deviation (s.d.). Assignments of the mice to treatments and selections of fields of microscopic inspection were made at random. Sample size (n) was constrained in most cases to 3 per treatment, as described in the figure legends. The small sample sizes, combined with no apparent data evidence of deviations from ANOVA assumptions (not shown), supported analysis using one-way ANOVA or two-way ANOVA, followed by Tukey's post hoc comparison test. In all cases, significant differences between groups were indicated by *P<0.05, **P<0.01, ***P<0.001 and ****P<0.0001, respectively. P≥0.05 was considered not statistically significant in all analyses (95% confidence level). Statistical analyses were performed using GraphPad Prism 9.0 software.

### Example 1: Preparation of Amphiphilic Copolymers for NP

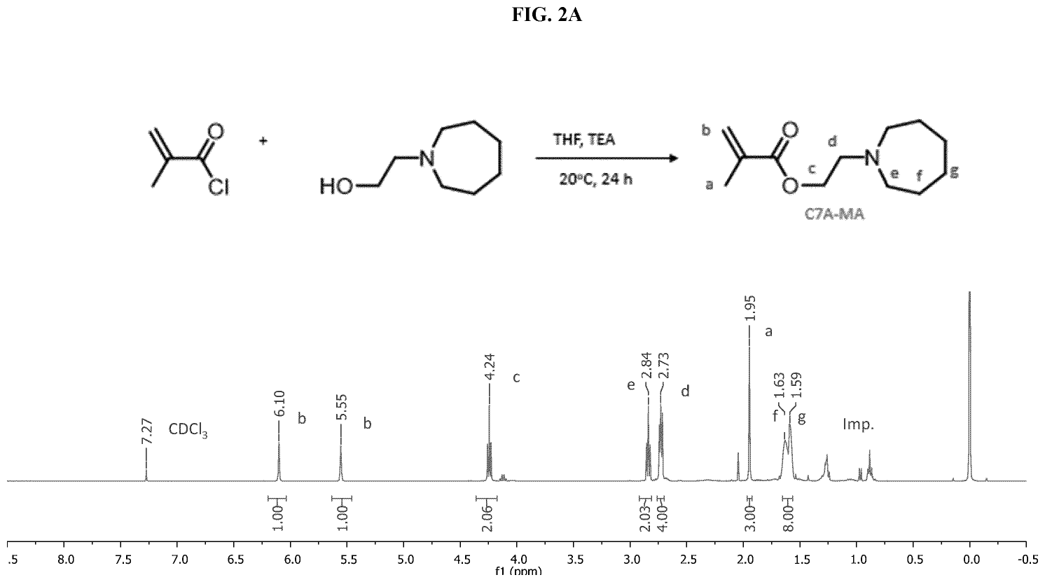

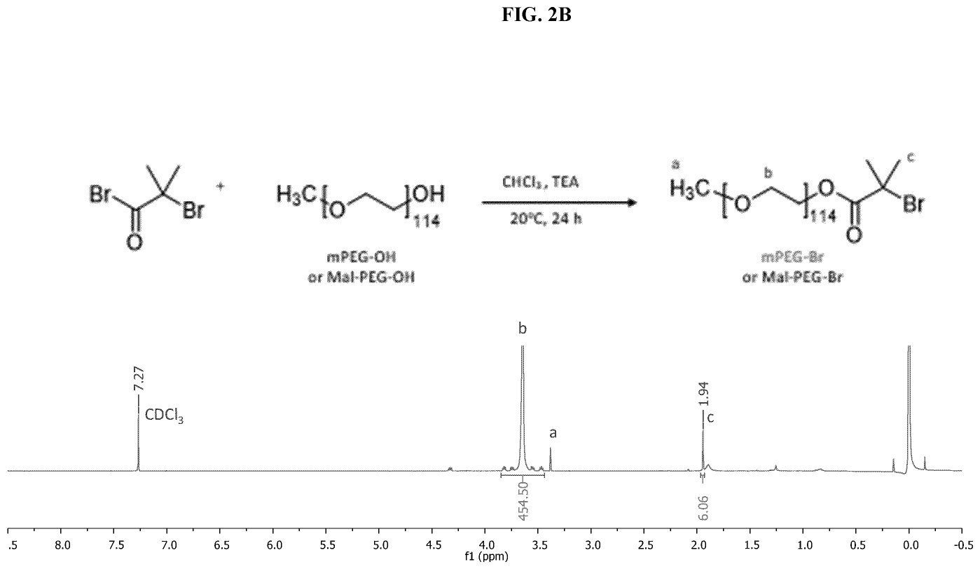

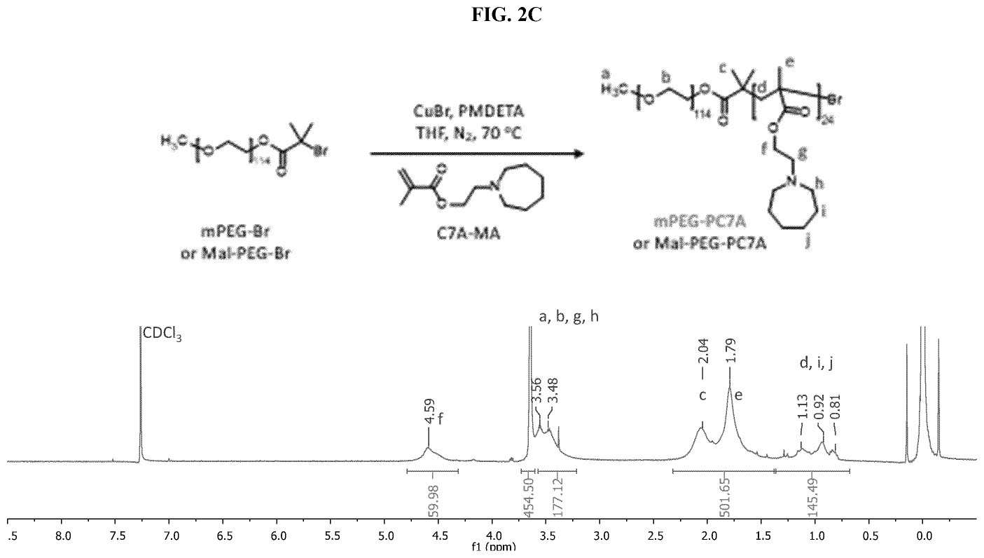

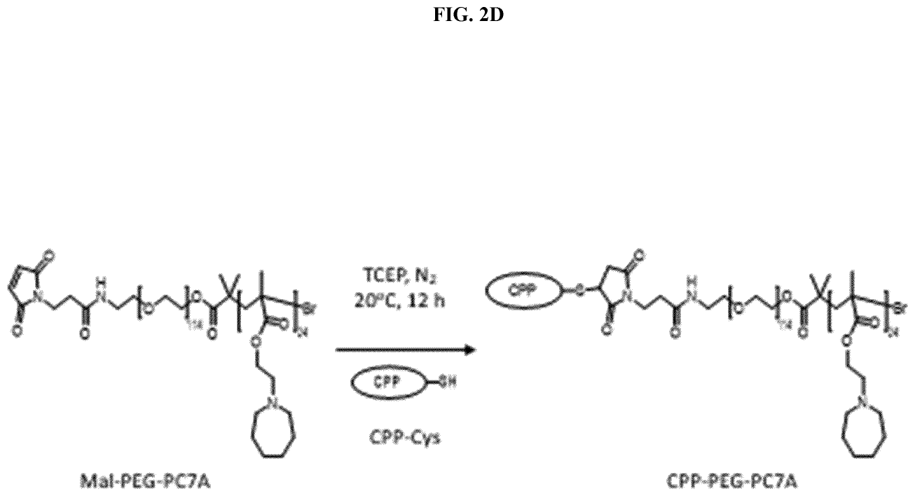

Synthesis of 2-(azepan-1-yl)ethyl methacrylate (C7A-MA). Triethylamine (7.0 g, 69 mmol) and 2-(azepan-1-yl)ethanol (5.0 g, 34.5 mmol) were dissolved in anhydrous THE (100 mL) and cooled on ice. Methacryloyl chloride (4.0 g, 38.5 mmol) in anhydrous THE (15 mL) was added dropwise into the previous solution. The reaction was stirred at 20° C. for 24 h and then the reaction mixture was filtered to remove triethylamine hydrochloride salt. The resulting filtrate was condensed by rotary evaporation and further purified by column chromatography using hexane/ethyl acetate (3/1, v/v) as the eluent. The product was obtained by removing solvents in vacuo and characterized by ¹H NMR (CDCl₃, 400 MHz). (FIG. 2A).

Synthesis of methoxy-/maleimide-poly(ethylene glycol)-2-bromoisobutyrate (mPEG-Br and Mal-PEG-Br). mPEG-OH (1.0 g, 0.2 mmol) was dissolved in chloroform (30 mL) followed by the addition of triethylamine (1 mL). The solution was cooled on ice, and 2-bromoisobutyryl bromide (0.3 mL, 2.4 mmol) in chloroform (10 mL) was added dropwise. The mixture was stirred at 20° C. for 24 h. The solution was washed with saturated brine (40 mL) three times and dried with anhydrous MgSO₄ for 12 h. The supernatant was then concentrated to 5 mL by rotary evaporation and the resulting solution was precipitated in 45 mL of ice-cold diethyl ether to yield the final product (i.e., mPEG-Br). Mal-PEG-Br was synthesized following the same protocol except Mal-PEG-OH was used instead of mPEG-OH. The products were obtained by removing solvents in vacuo and characterized by ¹H NMR (CDCl₃, 400 MHz). (FIG. 2B-).

Synthesis of methoxy-/maleimide-poly(ethylene glycol)-b-poly(2-(azepan-1-yl)ethyl methacrylate) (mPEG-PC7A). mPEG-PC7A was synthesized by atom transfer radical polymerization (ATRP) using mPEG-Br as a macroinitiator. C7A-MA (0.41 g, 2 mmol), CuBr (2.8 mg, 20 μmol), and mPEG-Br (0.2 g, 40 μmol) were dissolved in 1.0 mL of anhydrous THF. After three cycles of freeze-pump-thaw, PMDETA (3.4 mg, 20 μmol) was added. The polymerization was carried out at 70° C. for 20 h. The reaction mixture was dissolved by adjusting pH to ˜4 with 1 M HCl aqueous solution and dialyzed with a dialysis membrane tubing (molecular weight cut-off, MWCO˜3500 Da) against ultrapure water for 72 h to yield purified mPEG-PC7A. Mal-PEG-PC7A was synthesized following the same protocol using Mal-PEG-Br instead. The products were obtained by lyophilization and characterized by ¹H NMR (CDCl₃, 400 MHz). (FIG. 2C).

Synthesis of cell-penetrating peptide-conjugated poly(ethylene glycol)-b-poly(2-(azepan-1-yl)ethyl methacrylate) (CPP-PEG-PC7A). CPP was conjugated to Mal-PEG-PC7A through a maleimide-thiol Michael reaction. Cys-TAT(47-57) (1.82 mg, 1.1 μmol) and TCEP (1.58 mg, 5.5 μmol) were dissolved in anhydrous DMF (1 mL). Subsequently, the Mal-PEG-PC7A (10.0 mg, 1.0 μmol) solution (anhydrous DMF (1 mL)) was mixed with this solution. The reaction was conducted under nitrogen at 20° C. for 12 h. Thereafter, the solution was dialyzed against ultrapure water (MWCO˜3500 Da) for 72 h, and the final product was collected by lyophilization. (FIG. 2D).

### Example 2: Preparation of NHEJ-NPs and HDR-NPs

Cas9 RNP was formed by complexing sNLS-SpCas9-sNLS nuclease (Aldevron, LLC) with sgRNA (Integrated DNA Technologies, Inc.) at a molar ratio of 1/1 at 20° C. for 5 min. For HDR-NP synthesis, ssODN (Integrated DNA Technologies, Inc.) was mixed with Cas9 RNP at a molar ratio of 1/1. The payload solution (5 mg/mL) was then diluted with 100 mM Tris HCl buffer solution to 2.5 mg/mL. Thereafter, the mPEG-PC7A aqueous solution (20 mg/mL) was mixed with the payload solution at 0.625/1 (vol/vol). The resulting mixture solution was vortexed for 1 min, incubated at 20° C. for 15 min, diluted with Tris HCl buffer solution, and then vortexed again for 1 min. After another incubation at 20° C. for 15 min, solutions containing NHEJ-NPs or HDR-NPs were directly used for cell treatments or animal injections without further purification. CPP-NHEJ-NP was prepared by using 50% CPP-PEG-PC7A with 50% mPEG-PC7A in molar ratio following the same process. The final NHEJ-NP or HDR-NP solution was isotonic (in 150 mM Tris HCl) and contained 1.0 mg/mL Cas9 nuclease. For the preparation of hypotonic NHEJ-NP, the final NHEJ-NP solution was in 15 mM Tris HCl. Sequences of sgRNAs and ssODNs used in this work were summarized in Tables 2 and 3.

TABLE 2
Protospacers of sgRNAs.

| Gene | Protospacer (5′ to 3′) |
|---|---|
| GFP | GCACGGGCAGCTTGCCGG |
| BFP | GCTGAAGCACTGCACGCCAT |
| SV40 PolyA | AAGTAAAACCTCTACAAATG |
| (Ai14) | |
| Dmd | TCTTTGAAAGAGCAATAAAA |

TABLE 3
Sequences of ssODNs.

| Gene | Sequence (5′ to 3′) |
|---|---|
| BFP | AAGTTCATCTGCACCACCGGCAAGCTGCCGGTGCCCTGGCCCACC |
| | CTCGTGACCACCCTGACGTACGGCGTGCAGTGCTTCAGCCGCTAC |
| | CCCGACC |
| Dmd | TGATATGAATGAAACTCATCAAATATGCGTGTTAGTGTAAATGAA |
| | CTTCTATTTAATTTTGAGGCTCTGCAAAGTTCTTTAAAGGAGCAG |
| | CAGAATGGCTTCAACTATCTGAGTGACACTGTGAAGGAGATGGCC |
| | AAGAAAGCACCTTCAGAAATATGCCAGAAATATCTGTCAGAATTT |

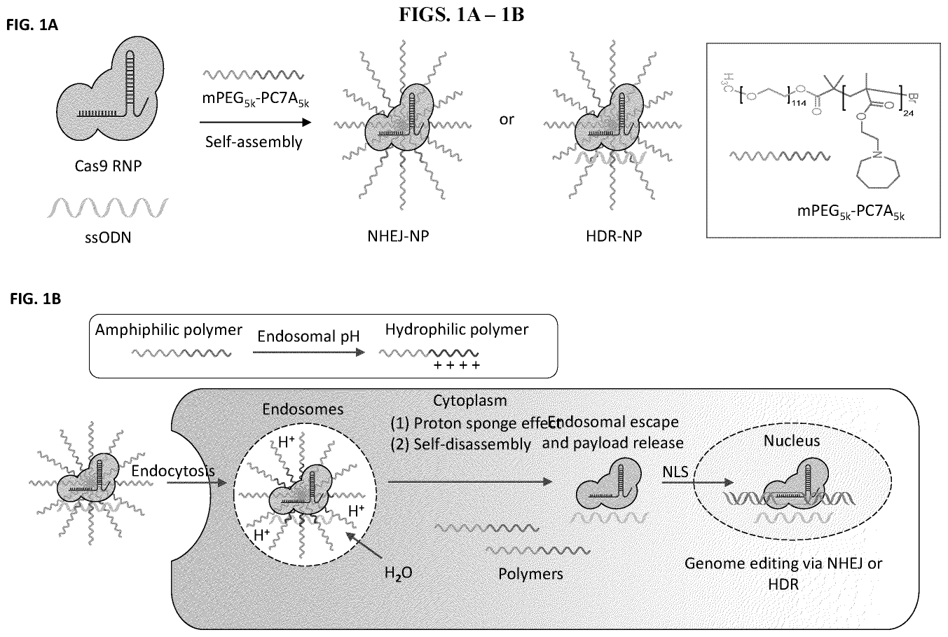

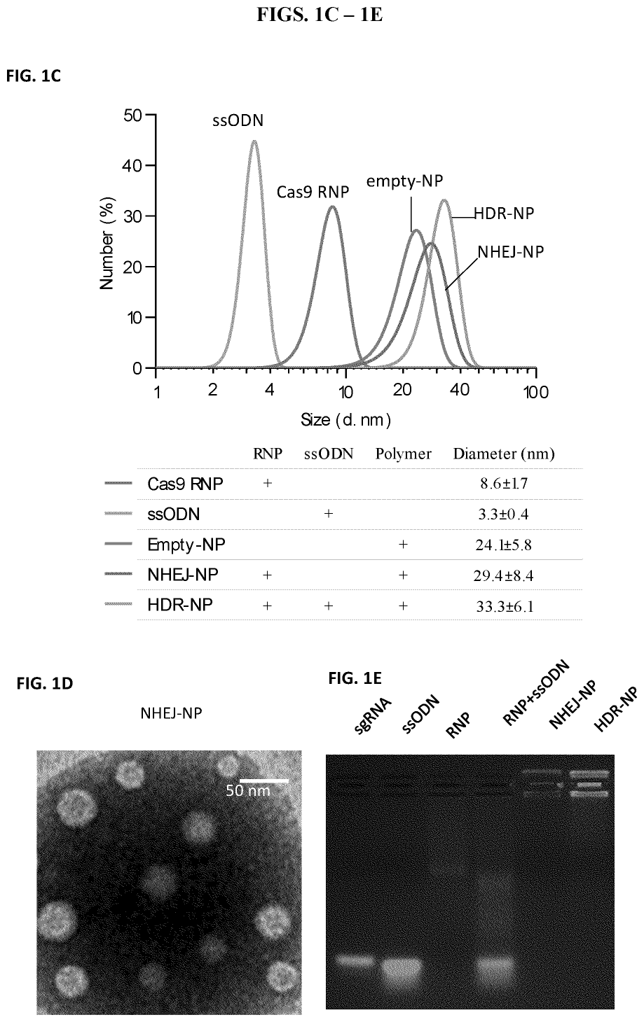

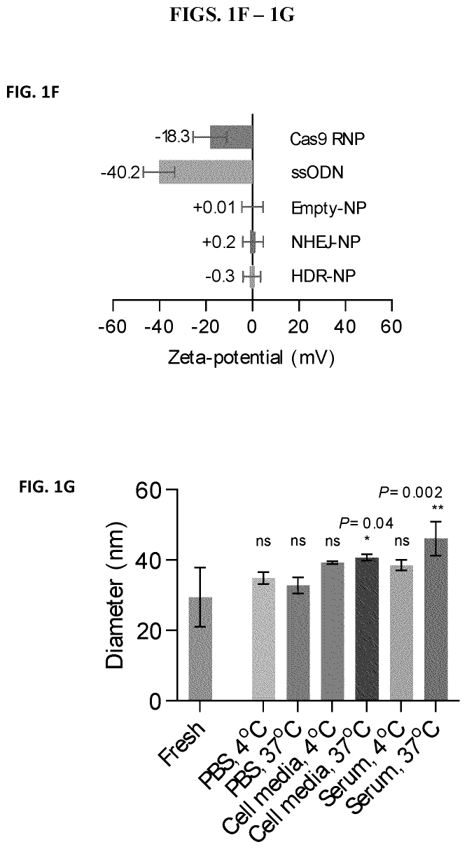

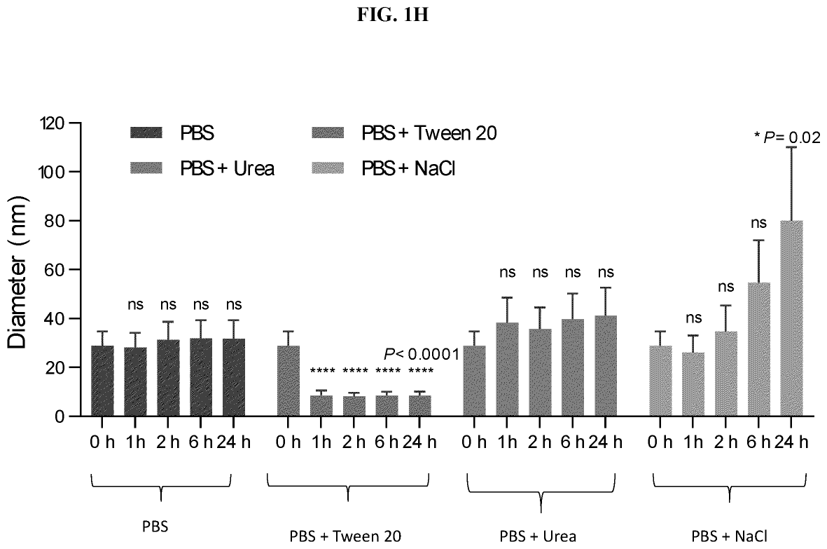

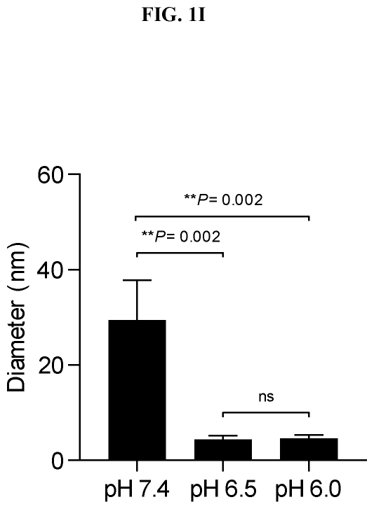

mPEG-PC7A self-assembled with the Cas9 RNP and the ssODN for delivery. mPEG-PC7A efficiently complexes with either Cas9 RNPs (termed as “NHEJ-NP”) or Cas9 RNPs with ssODNs (termed as “HDR-NP”) to form nanoparticles via a straightforward self-assembly process (FIG. 1A). Upon endocytosis, mPEG-PC7A becomes highly protonated in the acidic endosomal/lysosomal environment at pH 5.5-6.5, converting the hydrophobic PC7A polymer segment to a highly cationic and hydrophilic PC7A segment. This dramatic change in charges and hydrophilicity facilitates the endosomal escape of the payloads via the “proton sponge” effect, the NP disassembly, and the release of the payloads into the cytoplasm. Thereafter, facilitated by the nuclear localization signals (NLS), the NLS-conjugated Cas9 RNP and the ssODN can enter the cell nucleus for genome editing via NHEJ or HDR (FIG. 1b).

Transmission electron microscopy (TEM) and dynamic light scattering (DLS) revealed well-defined spherical NP shape and average hydrodynamic diameters of 29.4 nm (PDI of 0.18) and 33.3 nm (PDI of 0.21) for NHEJ-NP and HDR-NP, respectively (FIG. 1C, 1D). NHEJ-NP and HDR-NP with zeta-potentials at +0.2 and −0.3 mV suggested the surface charge of payloads (i.e., Cas9 RNPs and ssODNs) were completely shielded, demonstrating efficient payload encapsulation and the formation of NPs (FIG. 1F). Gel electrophoresis further verified payload encapsulation (FIG. 1E), where no observable band migration was found from both types of NPs, in strong contrast to the naked payloads. To determine the interactions between the polymer and Cas9 RNP, Tween 20, NaCl, and urea were mixed with NHEJ-NP, and the size variations of NHEJ-NP were monitored by DLS²⁵. As shown in FIG. 1H, the size of NHEJ-NP in the presence of Tween 20 was reduced from 28.8 nm to 8.5 nm after one hour, suggesting rapid and complete NP dissociation attributed to competitive hydrophobic interactions. NHEJ-NP swelled in the NaCl solution over time and increased to 80 nm after 24 h, indicating that electrostatic interaction plays a role in the formation of NHEJ-NP. In contrast, urea, which disturbs hydrogen bonding, did not cause any significant size change. These results support our hypothesis that the interaction between mPEG-PC7A and Cas9 RNPs is a combination of hydrophobic and electrostatic interactions. The stability of NHEJ-NP was also examined. As shown in FIG. 1g, NHEJ-NP was dispersed in PBS, serum-containing cell culture medium, or serum, and stored at 4° C. or 37° C. for 24 h. NHEJ-NP remained stable in most conditions but slightly swelled in the presence of serum at 37° C. In contrast, acidic environments (e.g., pH 6.0 and 6.5) quickly triggered the disassembly of NHEJ-NP and its hydrodynamic diameter was reduced to ˜4 nm (FIG. 1i). This finding supports our hypothesis that the nanosystem can disassemble in the acidic endosomal compartment to release the payloads.

### Example 3: NHEJ-NP and HDR-NP Delivered Genome Editors and Edited Human Cells In Vitro Via NHEJ and HDR

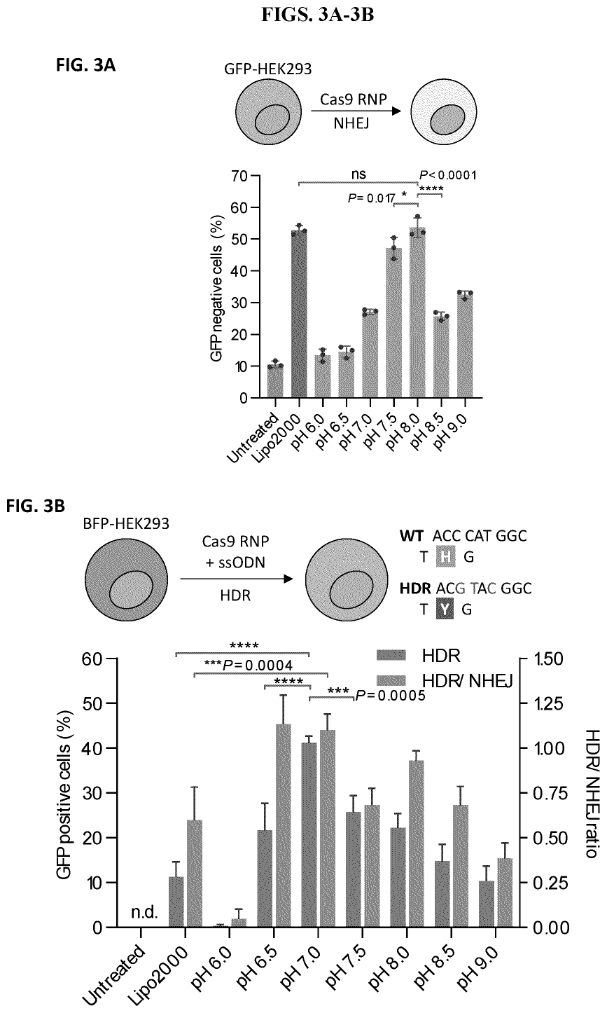

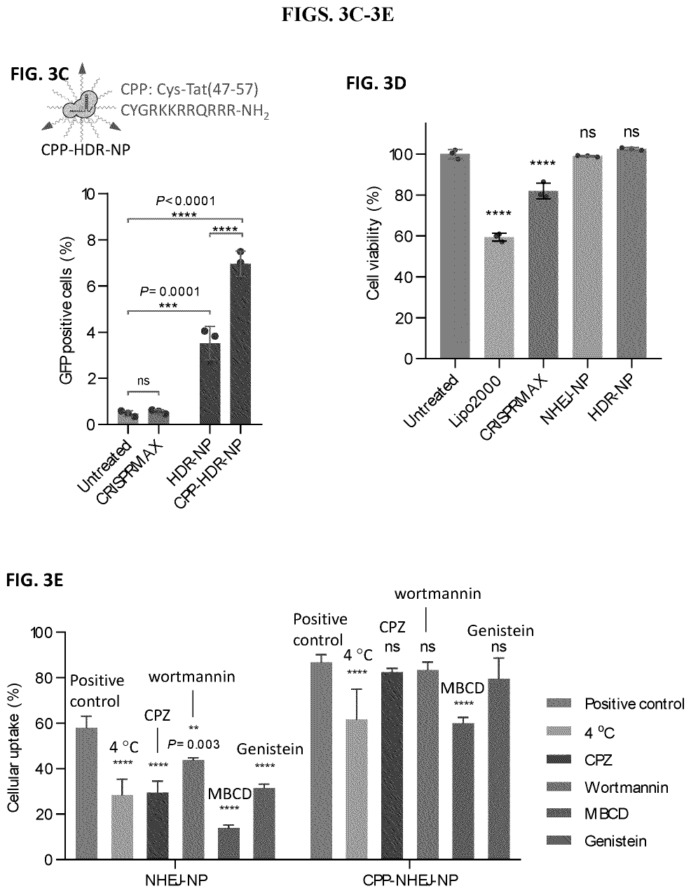

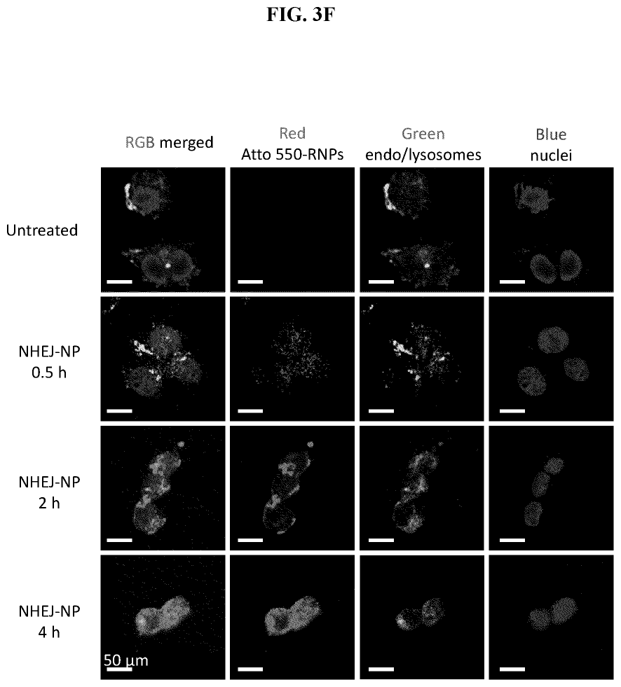

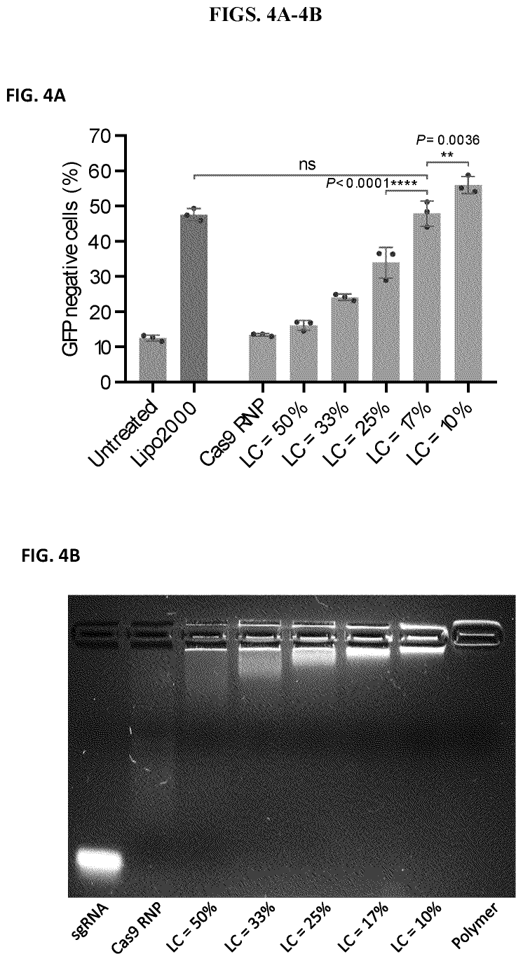

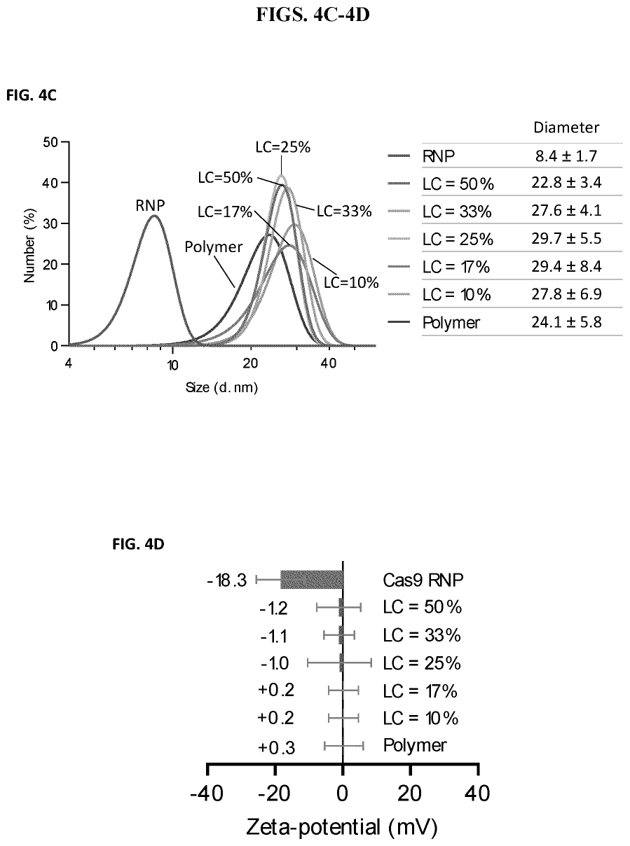

The formulations of NHEJ-NP were first optimized using green fluorescent protein (GFP) expressing human embryonic kidney cells (GFP-HEK293), where successful gene disruption leads to silence of GFP expression and can be detected by flow cytometry. We hypothesized that the degree of protonation for the tertiary amine groups in the polymer is crucial to encapsulate the Cas9 RNP as they are cationic and hydrophilic when protonated and hydrophobic when deprotonated. The pH value for nanoparticle formation was observed to play a key role in delivery efficiency. A series of NHEJ-NPs were prepared at different pH values and used to treat the cells (FIG. 3A). NHEJ-NP formed at pH 8.0 exhibited comparable gene editing efficiency with Lipofectamine 2000. NHEJ-NPs formed at pH<8.0 showed lower editing efficiencies, possibly due to poor nanoparticle stability resulting from insufficient hydrophobic interactions between the polymer and Cas9 RNP. Similarly, NHEJ-NPs prepared at pH>8.0 had lower gene editing efficiencies, possibly due to lower RNP encapsulation efficiency resulting from insufficient cationic charges of the PC7A polymer at a higher pH (hence a higher degree of deprotonation). Additionally, a series of NHEJ-NPs with different polymer/Cas9 RNP weight ratios were prepared in order to optimize the loading content (LC) of NHEJ-NP. Higher polymer/Cas9 RNP ratio led to less band migration in gel electrophoresis, suggesting more efficient encapsulation (FIG. 4B). Based on this study, considering both genome editing efficiency and payload loading content, polymer/Cas9 RNP ratio at 5/1 (LC %=17%) was selected for further studies.

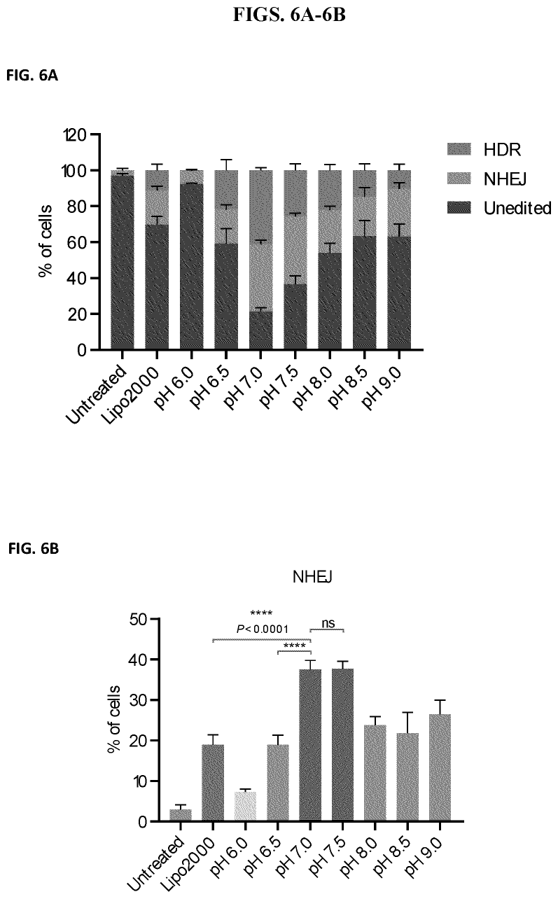

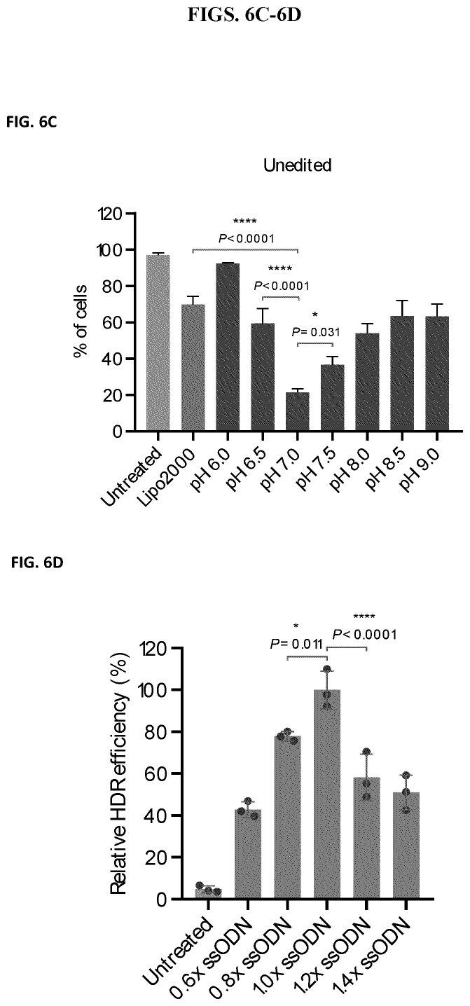

The formulation and editing efficiency of HDR-NPs were evaluated using blue fluorescent protein (BFP) expressing HEK293 (BFP-HEK293) cells. BFP-HEK293 cells contain a missense Y66H mutation of GFP. Therefore, gene correction via HDR (correcting CAT to TAC) leads to GFP expression and gene disruption via NHEJ leads to silence of BFP expression. The HDR and NHEJ efficiency can be thus quantified by flow cytometry. Similar to NHEJ-NP, an optimal pH value was determined for the HDR-NP preparation (FIG. 3B). HDR-NP formed at pH 7.0 exhibited a significantly higher HDR efficiency than Lipofectamine 2000, as measured by flow cytometry. As expected, the optimal pH for preparing HDR-NP was lower than the one for NHEJ-NP because ssODNs, 100-nt oligonucleotides bearing anionic charges, require more cationic (protonated) groups in the polymer to form stable HDR-NP. Notably, the HDR/NHEJ ratio induced by HDR-NP at pH of 7.0 (i.e., HDR/NHEJ=1.1) was about 1.8-fold higher than that of Lipofectamine 2000 (i.e., HDR/NHEJ=0.6), likely owing to more efficient codelivery of Cas9 RNP and ssODN at the same time (FIG. 6A). The optimal molar ratio of ssODN to Cas9 RNP was confirmed at 1/1, demonstrating the unbiased encapsulation of both payloads by HDR-NP under the optimal preparation condition (FIG. 6f). In terms of cell viability, neither NHEJ-NP nor HDR-NP exhibited cytotoxicity at their working concentration (12.5 μg/mL) in HEK293 cells, whereas Lipofectamine 2000 and Lipofectamine CRISPRMAX both showed significant cytotoxicity at the dosage used to deliver the same amount of the payload (FIG. 3D).

To demonstrate the capability of HDR-NPs in editing hard-to-transfect cells and the versatility of ligand conjugation to HDR-NP, HDR-NP was functionalized with cell-penetrating peptides (CPPs) (i.e., TAT (47-57)). CPPs were conjugated to the distal end of the PEG through maleimide-thiol coupling reactions (FIG. 2D). CPP-conjugation significantly enhanced (˜2-fold) the HDR efficiency of HDR-NP to 7.0% in BFP-expressing human embryonic stem cells (hESCs), as quantified by flow cytometry (FIG. 3C), while Lipofectamine CRISPRMAX can hardly edit hESCs.

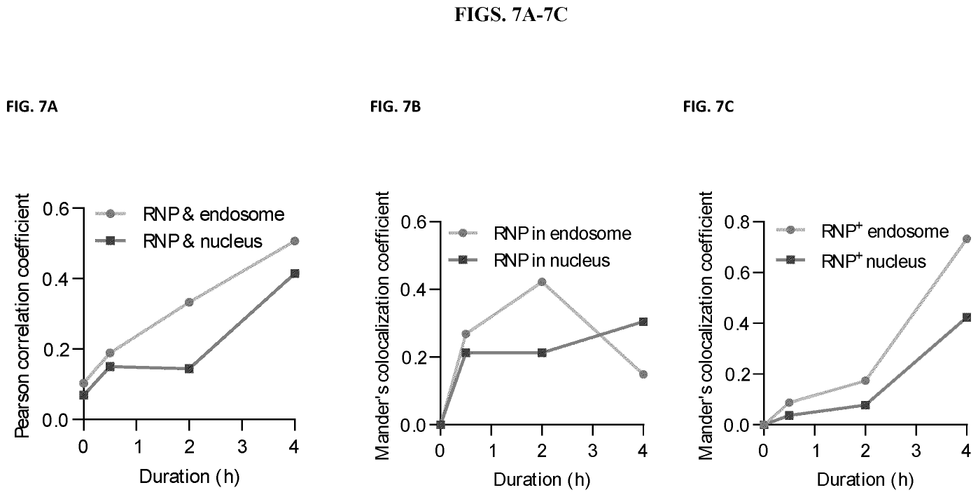

The cellular uptake behaviors of NHEJ-NP and CPP-NHEJ-NP in HEK293 cells at various conditions were studied. The gRNA used to form Cas9 RNP was fluorescently labeled by Atto 550, so the cellular uptake of NHEJ-NP and CPP-NHEJ-NP can be quantified by flow cytometry. To investigate the cellular uptake mechanism, HEK293 cells were treated with NHEJ-NP and CPP-NHEJ-NP respectively, with or without the presence of a series of endocytosis inhibitors at 37° C., or incubation at 4° C. (FIG. 3E). Chlorpromazine and wortmannin, both of which inhibit clathrin-mediated endocytosis, significantly reduced the cellular uptake of NHEJ-NP. Genistein, an inhibitor for caveolae-mediated endocytosis, and methyl-β-cyclodextrin, a molecule that depletes cholesterol and thus limits cell membrane fluidity, or incubation at 4° C., a temperature at which energy-dependent endocytosis is inhibited, also significantly reduced the cellular uptake of NHEJ-NP. This indicates that NHEJ-NP were taken up by cells via both clathrin- and caveolae-mediated endocytosis. However, the cellular uptake of CPP-NHEJ-NP was reduced ˜30% when cells were incubated at 4° C. alone or treated with methyl-β-cyclodextrin at 37° C., indicating that CPP conjugation altered the cellular uptake from endocytosis pathways to primary endocytosis-independent pathways^{11,26}. Furthermore, the subcellular localization of the Cas9 RNP delivered by NHEJ-NP in HEK293 cells was studied by confocal laser scanning microscopy (CLSM, FIG. 3F). It was found that Atto 550-labeled Cas9 RNPs (red fluorescence) could escape from endosomes/lysosomes (green fluorescence) barely 0.5 h after the treatment. More RNPs were found in the cytosol, not overlapping with endosomes/lysosomes, 2 h, and 4 h post-treatment, suggesting efficient payload escape from endosomes/lysosomes facilitated by the pH-responsive polymer. Nuclear transport of Cas9 RNPs was also visualized by considerable overlapping of Cas9 RNPs (red fluorescence) with cell nuclei (blue fluorescence) at 4 h post-treatment. The CLSM images were then quantitatively analyzed to calculate the Pearson correlation coefficient and Mander's overlap coefficient as proof for efficient endosome/lysosome escape and nuclear transport of Cas9 RNPs (FIGS. 7A-7C)^{27,28}. Collectively, our cellular uptake and subcellular trafficking studies support the mechanism outlined in FIG. 1B.

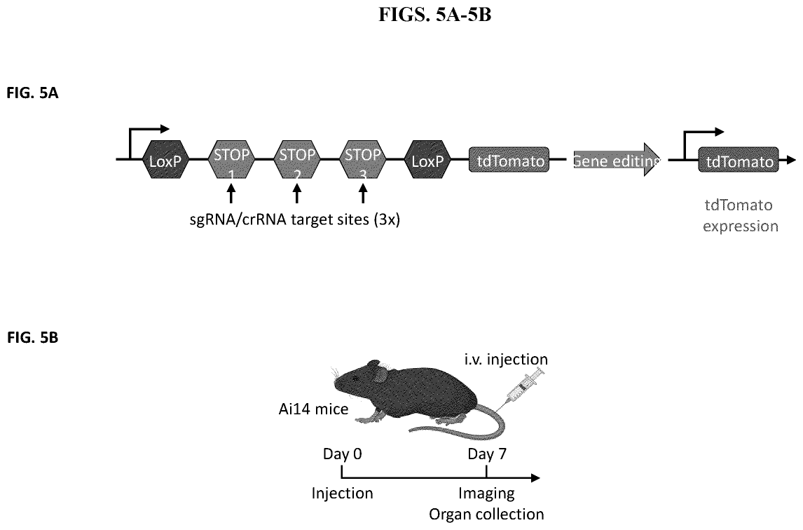

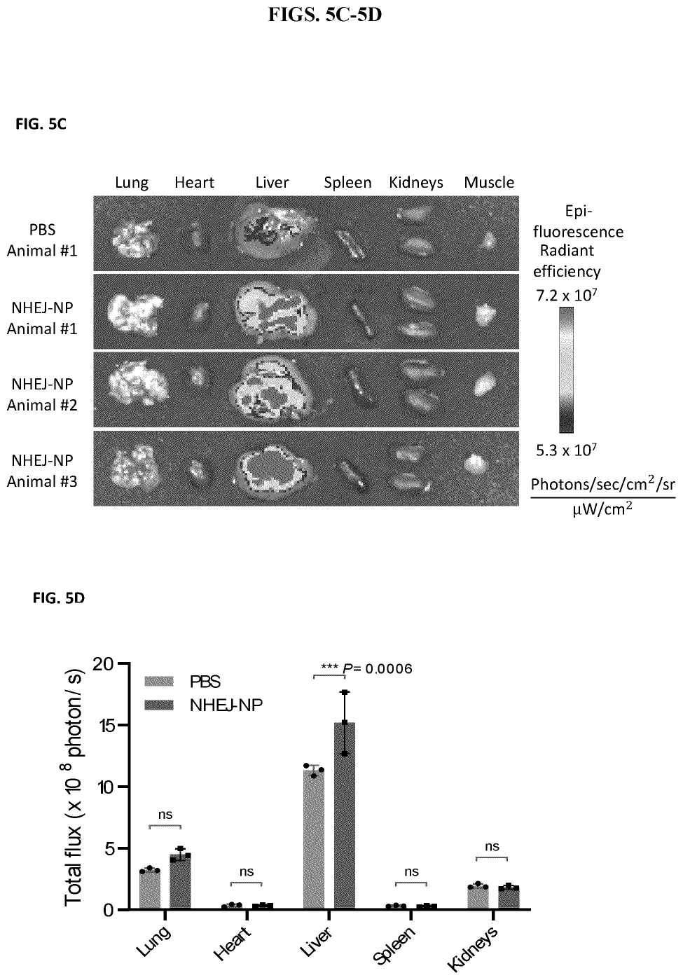

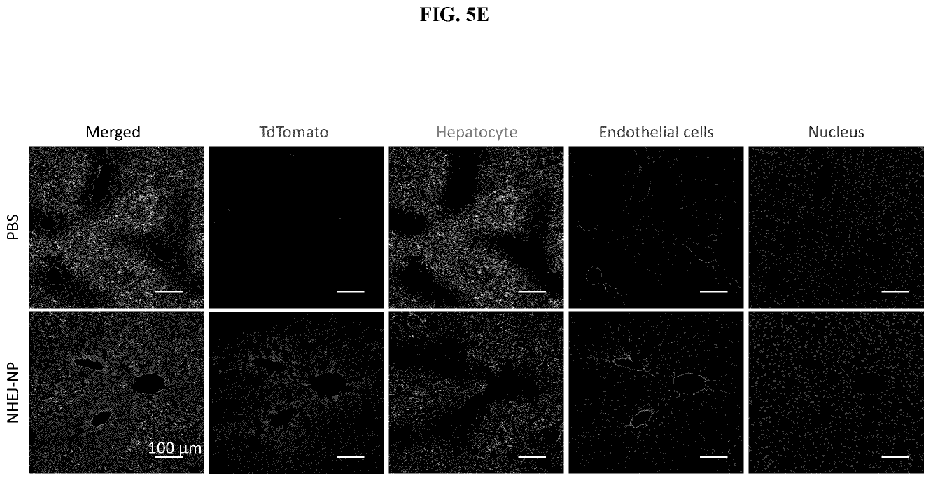

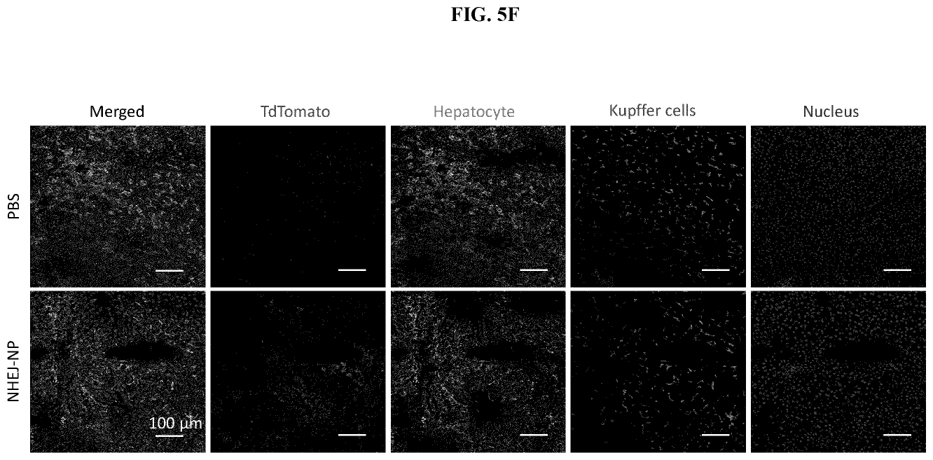

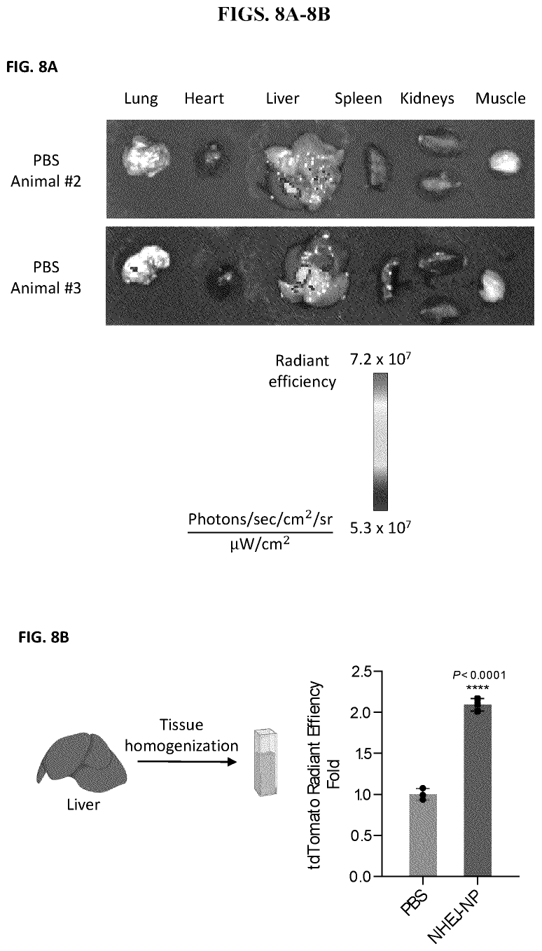

NHEJ-NP edited mouse livers after intravenous injection. The in vivo gene editing efficiency of intravenously injected NHEJ-NP was investigated using transgenic Ai14 mice. The genome of the Ai14 mouse contains a LoxP-flanked stop cassette that prevents downstream tdTomato expression (FIG. 5A). The stop cassette is composed of three SV40 polyA transcription terminators that can be targeted by Cas9 RNP. Successful gene editing leads to the excision of the SV40 polyA genetic elements and the expression of tdTomato. Therefore, the gene editing efficiency can be evaluated through tdTomato expression, although tdTomato activation requires removal of at least two of the three SV40 polyA repeat sequences and thus underreports the gene editing efficiency.^{[13]} The Ai14 mice were intravenously injected with NHEJ-NP on Day 0, and their major organs and blood were collected on Day 7 (FIG. 5B). NHEJ-NP targeting SV40 polyA sequences induced significant elevation of tdTomato fluorescence in the livers where gene editing happened, as observed by in vivo imaging system (IVIS) before and after tissue homogenization (FIGS. 5C, 8A, 8B). Immunofluorescence staining demonstrated abundant tdTomato expression in hepatocytes, endothelial cells, and Kupffer cells in the liver (FIGS. 5E, 5F).

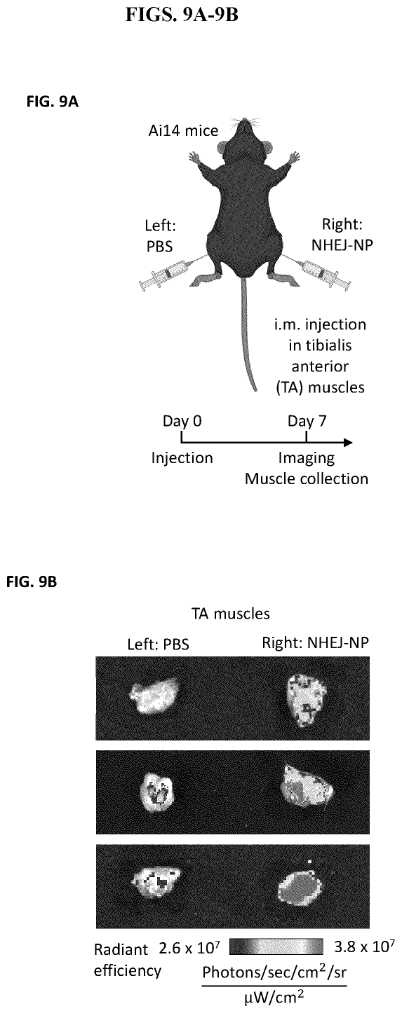

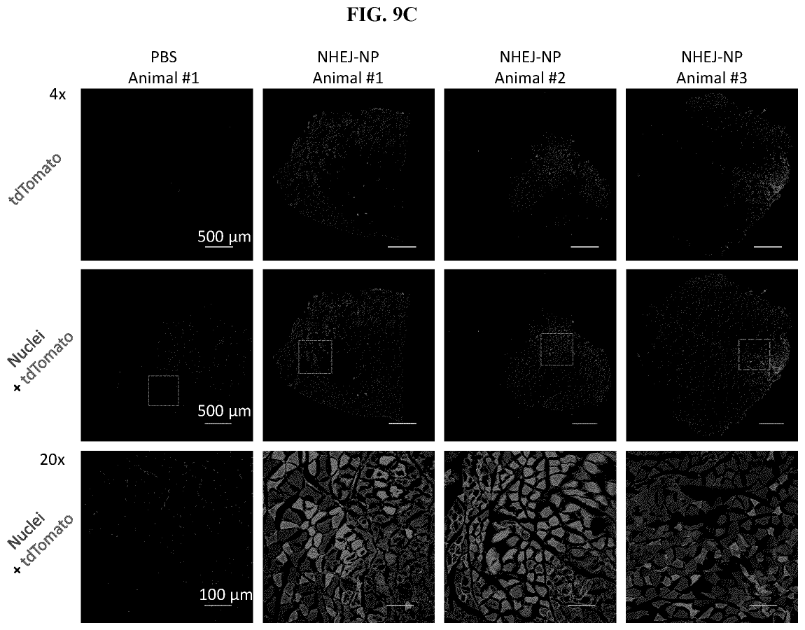

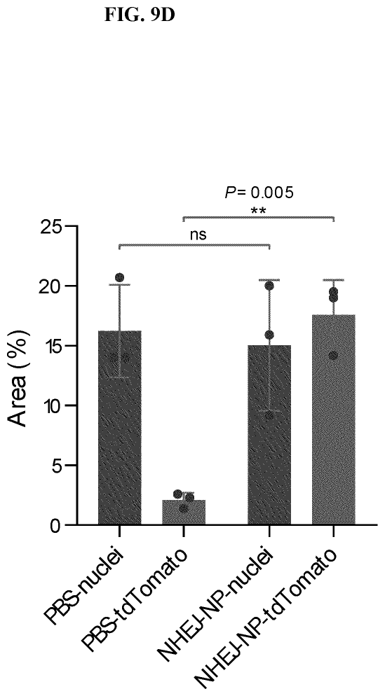

NHEJ-NP edited mouse muscles via intramuscular injection. The in vivo editing efficiency of NHEJ-NP in skeletal muscles was also investigated using Ai14 mice. For each Ai14 mouse, PBS and NHEJ-NP were intramuscularly injected in its left and right tibialis anterior muscle, respectively. Tibialis anterior muscles were collected and observed by IVIS to evaluate the fluorescence from tdTomato expression (FIGS. 9A, 9B). In contrast with the PBS-injected muscle from the same mouse, the NHEJ-NP-injected muscle exhibited a considerable amount of tdTomato fluorescence. The editing efficiency was further studied by immunofluorescence staining (FIGS. 9C, 9D). NHEJ-NP led to efficacious tdTomato expression in ˜16% of the muscle area, with substantial gene editing induced in myofibers.

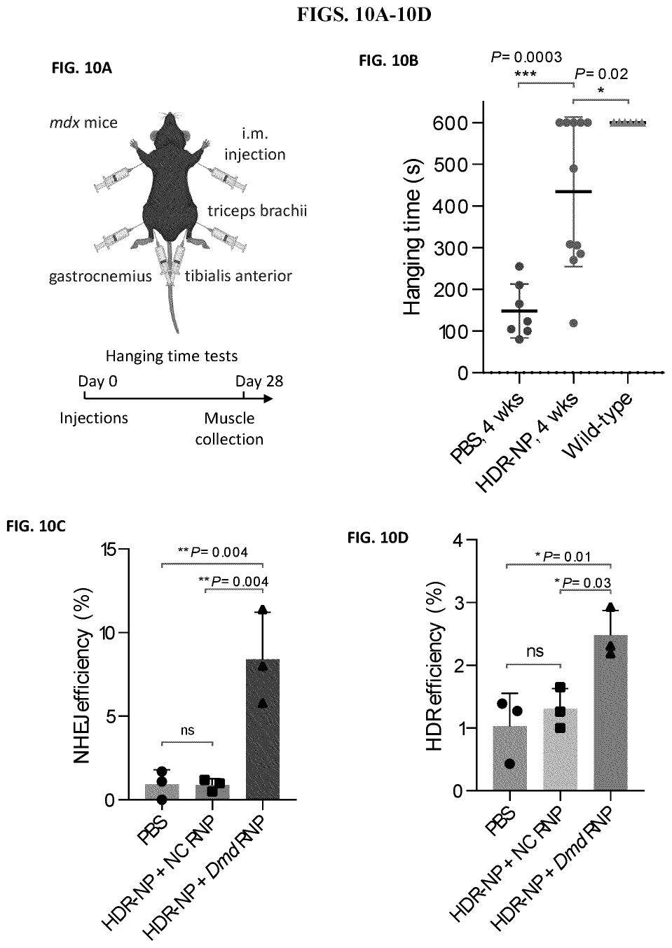

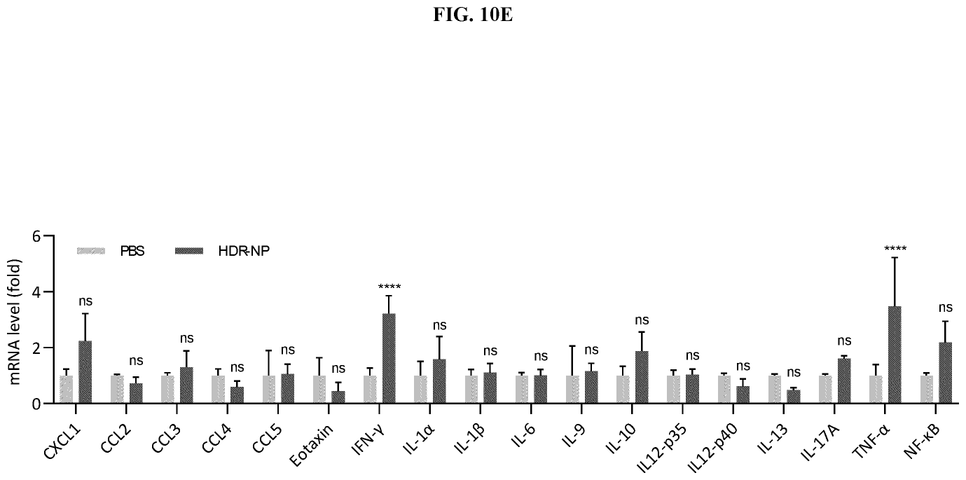

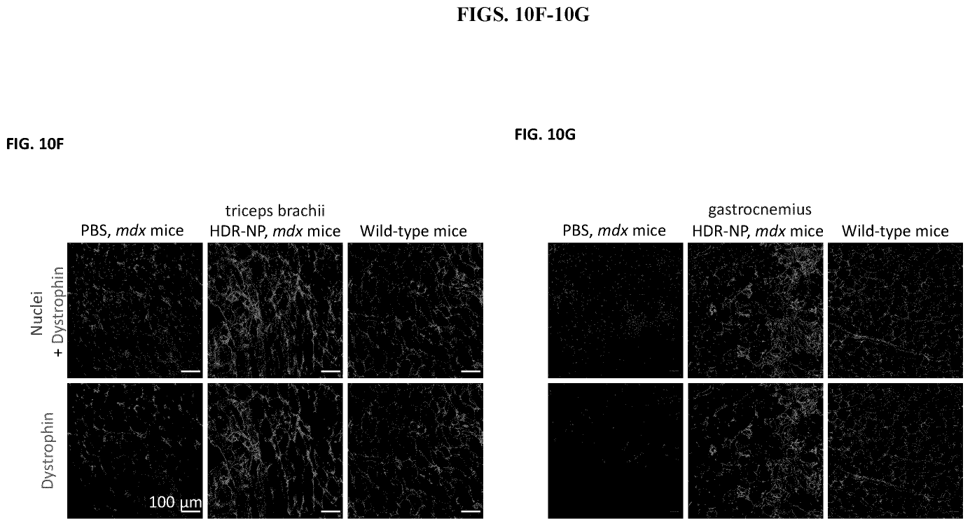

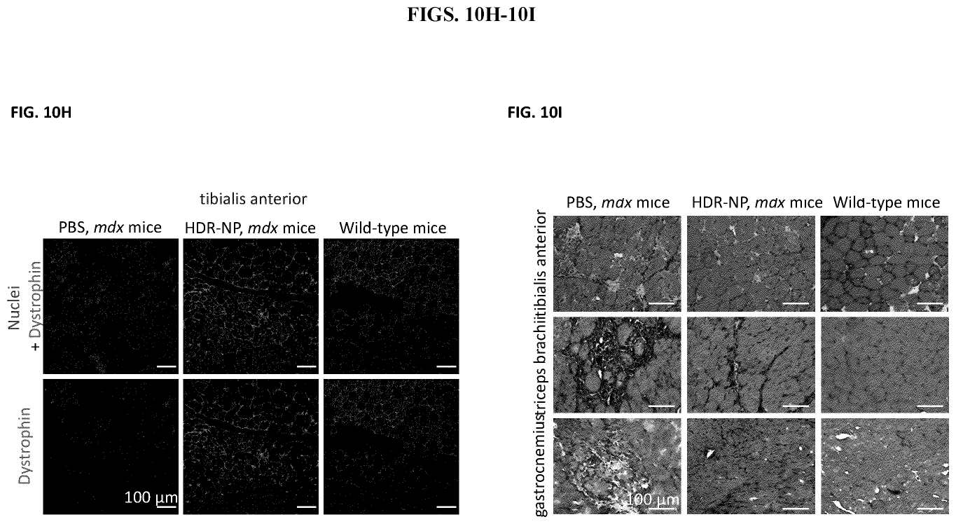

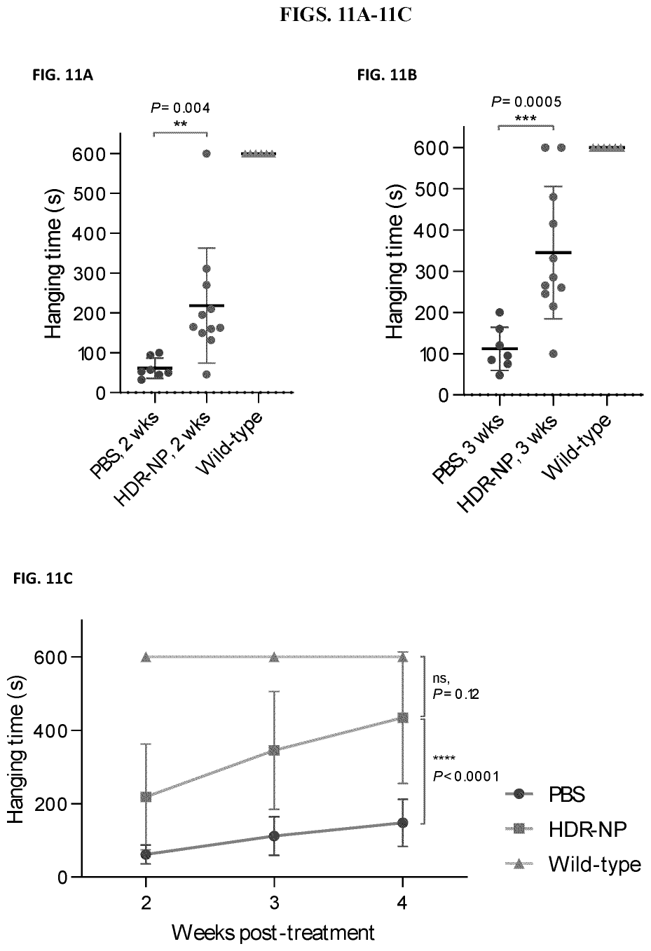

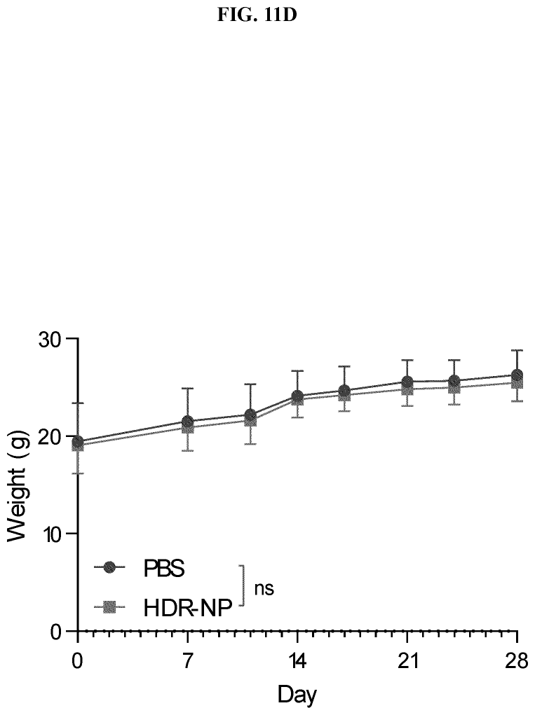

HDR-NPs induced in vivo editing for DMD treatment in mdx mice. Experiments were conducted to determine the potential of HDR-NPs as a therapeutic strategy for Duchenne muscular dystrophy (DMD), a notorious genetic disease affecting approximately 1 in 3,500 male births³⁰. The mdx mouse (C57BL/10ScSn-Dmd^{mdx}/J) possesses a nonsense mutation in exon 23 of the Dmd gene. Effective gene editing through NHEJ or HDR can disrupt or correct such a point mutation, restore dystrophin expression, and finally improve the muscle strength of the mdx mice. HDR-NPs were intramuscularly injected into both sides of the tibialis anterior, gastrocnemius, and triceps brachii muscles (total 6 injection sites per mouse) of 4-week-old male mdx mice, respectively (FIG. 10A). PBS-injected mdx mice and untreated wild-type mice were used as the negative and positive control group, respectively. Four-limb hanging tests of the mice were performed to evaluate the therapeutic benefits of HDR-NP. Notably, continuous improvement in muscle strength was observed for the HDR-NP-treated mdx mice during the 4-week experiment duration. Such an improvement suggests progressive rescue over time in skeletal muscles after gene editing (FIGS. 10B, 11A-11C). In particular, the HDR-NP-treated mdx mice showed 3-fold enhancement of the hanging time in contrast to the PBS-injected mdx mice 4 weeks after treatments, with 90% HDR-NP-treated mdx mice significantly improving the muscle strength compared with the PBS-injected group, and 45% HDR-NP-treated mdx mice performing similarly to wild-type mice (i.e., the hanging time ≥10 min). The muscles were harvested for analysis 4 weeks post-treatment. With immunofluorescence staining, robust dystrophin expression was observed in the subsarcolemmal region of myofibers in tibialis anterior, gastrocnemius, and triceps brachii muscles (FIGS. 10F-10H and data not shown). The distribution of dystrophin expression within the tibialis anterior muscle was also investigated by sectioning the muscle at different distances from the tendon. Notably, HDR-NP-mediated gene editing led to dystrophin restoration almost throughout the entire tibialis anterior muscle. Considerable dystrophin-positive signals were found in all tibialis anterior muscle sections collected at different distances away from the tendon (data not shown). Dystrophin expression was also observed abundantly in most area of the whole section of the HDR-NP-treated tibialis anterior muscle (data not shown). This can be attributed to the densely PEGylated NP surface and small NP size (33 nm diameter) that enable efficient diffusion of HDR-NP in the muscle. The pathology of muscle was further studied by trichrome staining (FIG. 10I). HDR-NP-treated mdx mouse muscles exhibited less muscle fibrosis and fewer pathological characteristics of muscular dystrophy, as well as interstitial fibrosis. The restoration of dystrophin expression is attributed to the gene editing on Dmd exon 23 via either HDR or NHEJ that corrects or disrupts the nonsense mutation³⁰. Sanger sequencing results revealed that 2.5% of HDR and 8.6% of NHEJ were induced by HDR-NP in the tibialis anterior muscles (FIGS. 10C, 10D). Although HDR-mediated gene correction cannot happen in postmitotic cells (e.g., myofibers)³¹, the gene editing via HDR in the muscle can be attributed to HDR that happened in myoblasts which can undergo mitosis. Myoblasts can proliferate and differentiate to myofibers during the muscle growth of mice and the recovery of muscle injuries⁸. Consequently, the corrected gene in myoblasts can finally function in newly generated myofibers to recover dystrophin expression. Collectively, these studies suggest that HDR-NP may offer a promising therapeutic approach to effectively treat DMD.

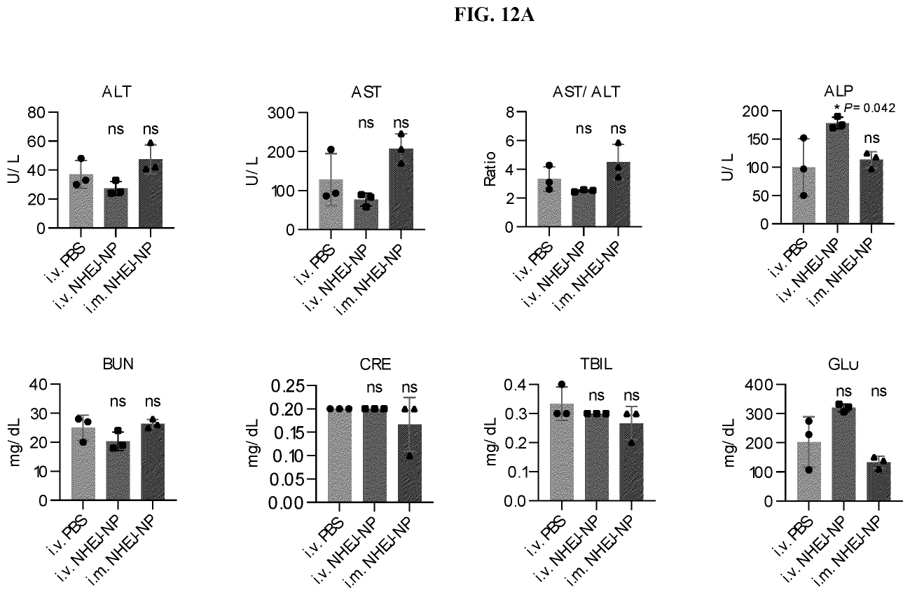

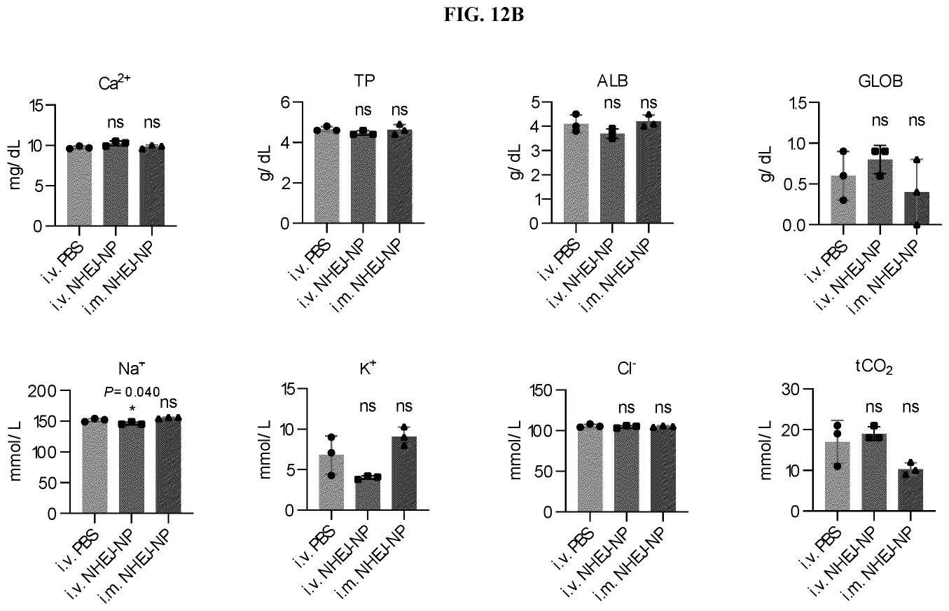

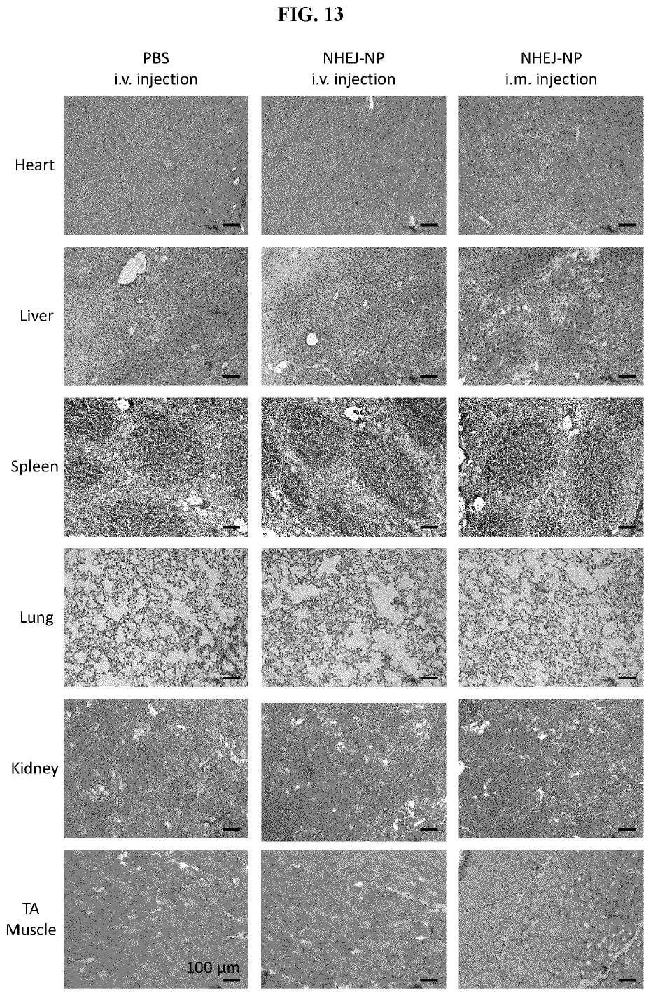

NHEJ-NP and HDR-NP exhibited good biocompatibility and negligible immunogenicity. The in vivo biocompatibility of NHEJ-NP was also evaluated after intravenous or intramuscular injections. A panel of blood biochemical parameters were measured using serum collected from Ai14 mice that received intravenous injection of PBS, intravenous injection of NHEJ-NP, and intramuscular injection of NHEJ-NP in tibialis anterior muscles, respectively. No significant variations were found in these biochemical parameters (FIG. 12). Major organs and muscles collected from these mice were observed via H&E staining (FIG. 13). Again, no sign of any systemic or local toxicity was found associated with NHEJ-NP treatments. Immunity against CRISPR-Cas9 which is a nuclease from Streptococcus pyogenes has been a concern for clinical translation^{8,32}.

To analyze the immune response generated by intramuscular administration of HDR-NP, a panel of inflammatory cytokine levels in the PBS- or HDR-NP-treated mdx mouse muscles were investigated by quantitative RT-PCR (FIG. 10E). HDR-NP did not induce significant changes in expression levels of most cytokines analyzed, although elevations of IFN-γ and TNF-α expression were detected. This can be attributed to the enhanced macrophage recruitment and/or activation for clearance of nanoparticles in the muscle, as previously reported^{8,33}.

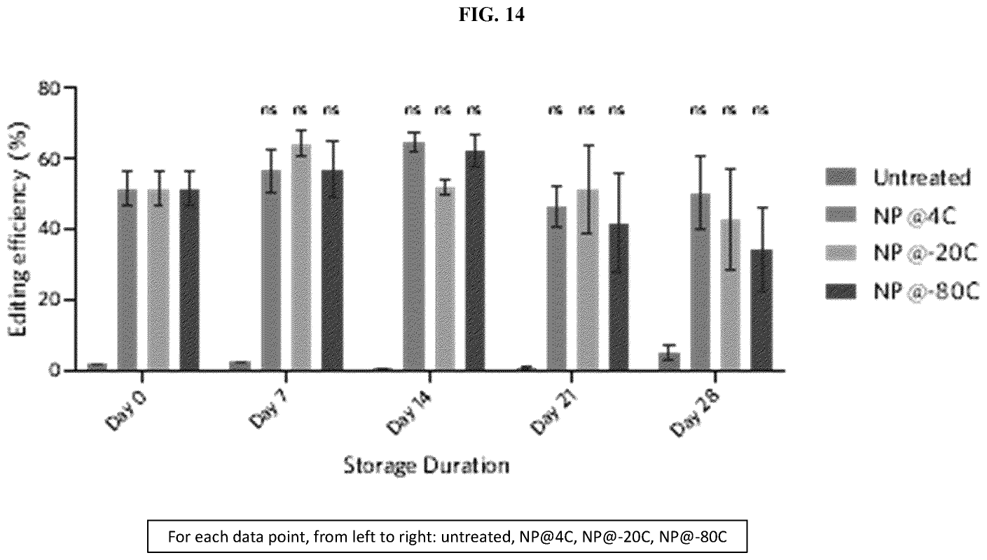

NHEJ-NP and HDR-NP Stability in Storage. Notwithstanding the ease of fabrication, the stability of pre-made NHEJ-NP stored at different temperatures was also tested. The editing efficiency of NHEJ-NP stored at 4° C., −20° C., or −80° C. was quantified in HEK293 cells weekly. It was found that NHEJ-NP could be stored at 4° C., −20° C., or −80° C. for 4 weeks without affecting its gene editing efficiency (FIG. 14). The stability of NHEJ-NP in serum-containing solutions was also examined. Thus, this unique RNP or RNP+ssODN delivery nanosystem is easy to synthesize, fabricate, and scale-up. Moreover, it can be easily stored and transported, making it particularly suitable for clinical translation.

### Example 4 In Vivo Gene Editing

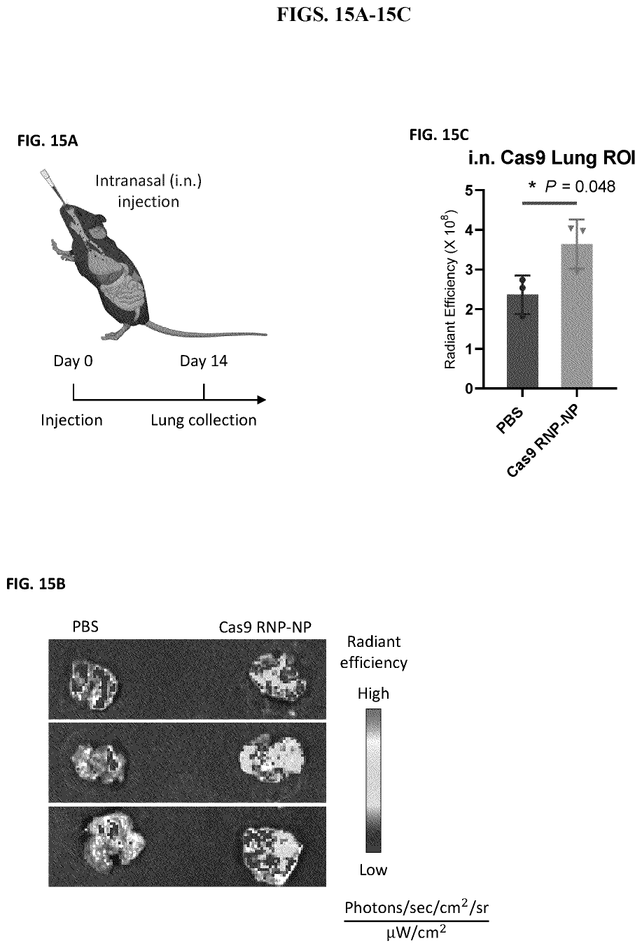

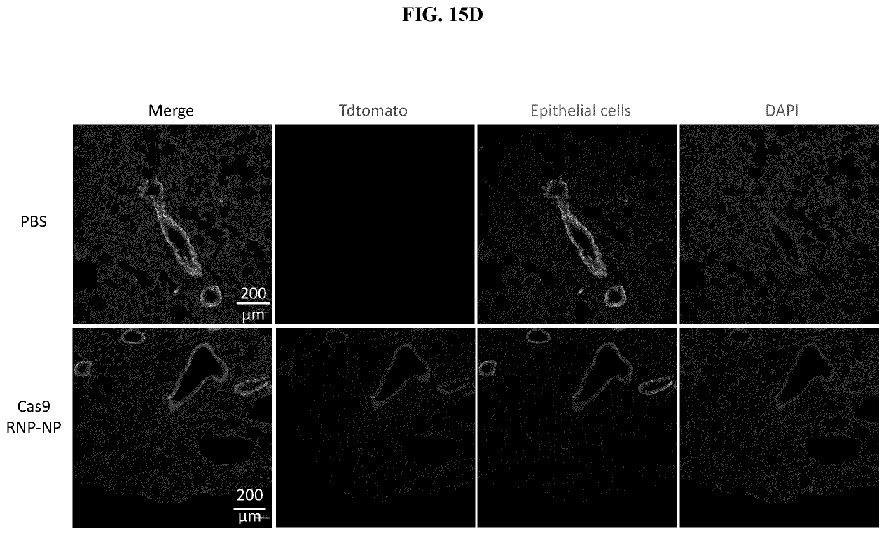

NHEJ-NP edited mouse lungs via intranasal injection. The in vivo gene editing efficacy in lung by intranasal injection of NHEJ-NP was studied using Ai14 mice. PBS and NHEJ-NP encapsulating Cas9 RNP was administrated into Ai14 mice by intranasal injection, respectively. Fourteen days after injection, the lungs were isolated and imaged. IVIS imaging showed obvious tdTomato fluorescence in the lungs of the NHEJ-NP-treated mice, which demonstrated effective gene editing in lungs (FIGS. 15A, 15B). Furthermore, the fluorescence intensity of NHEJ-NP-treated lungs was significantly higher than that of PBS-treated ones (FIG. 15C). The immunostaining of the cryosection of the lung tissue exhibited that the airway epithelial cells were edited effectively (FIG. 15D).

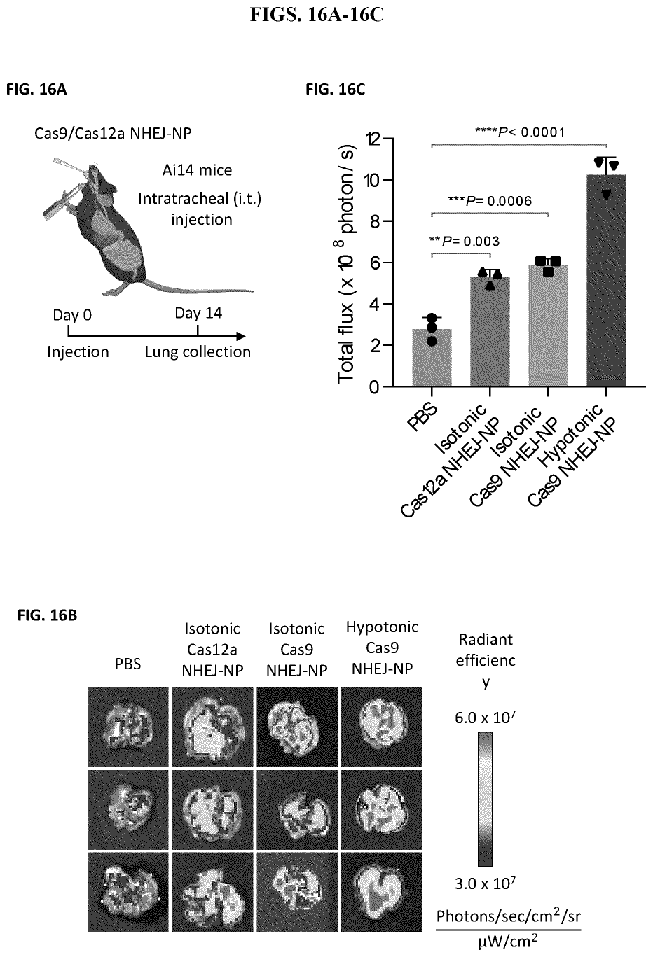

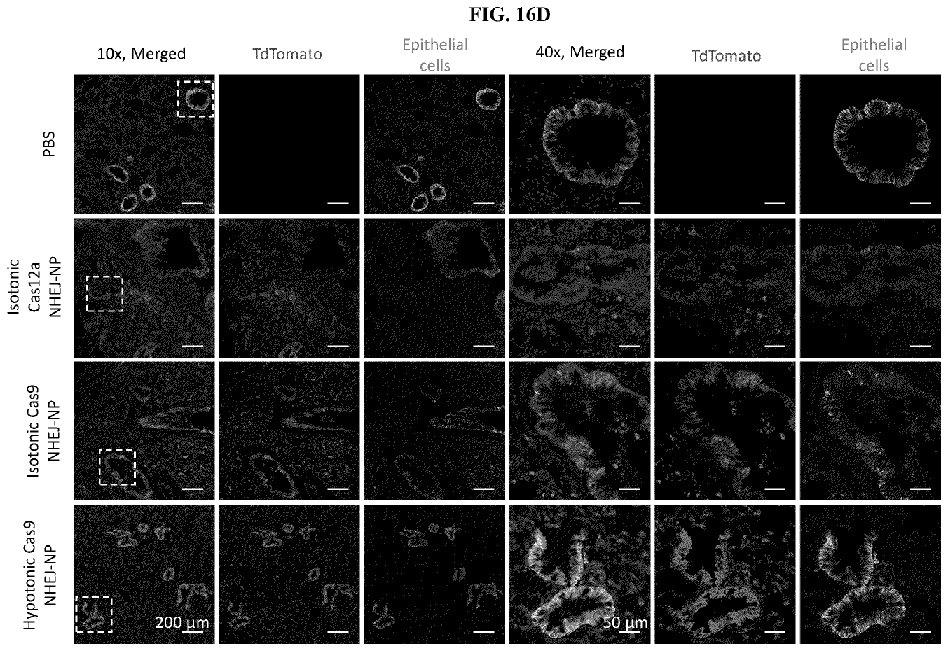

NHEJ-NP edited mouse lungs via intratracheal injection. Gene editing can be potentially used to treat genetic lung diseases such as cystic fibrosis, but it is challenging to deliver genome editor to lung epithelium due to the presence of the mucus layer that excludes delivery vectors.^{[14]} Due to their small sizes (i.e., 29.4 nm in diameter) and highly PEGylated and charge-neutralized surfaces, NHEJ-NP is desirable for mucus-penetration, thereby enabling gene editing in lung epithelium.^{[14c,15]} The in vivo editing efficiency of NHEJ-NP in lungs was investigated using Ai14 mice through intratracheal injection (FIG. 16A). Fourteen days after the injection, the mouse lungs were collected for analysis. NHEJ-NP targeting SV40 polyA sequences induced considerable expression of tdTomato in the lungs as observed by IVIS (FIGS. 16B, 16C). The tdTomato expression was also observed by immunofluorescence staining, where the gene editing-induced tdTomato expression was mostly found in airway epithelial cells (FIG. 16D), as well as alveolar type I cells and macrophages. To further enhance the gene editing efficiency in lung epithelium, NHEJ-NP was prepared in a hypotonic aqueous solution, because previous studies demonstrated that a hypotonic shock could force cells to undergo intensive endocytosis and thus enhance the cellular uptake of nanoparticles via the osmotically-driven regulatory volume decrease mechanism.^{[16]} It was found that hypotonic NHEJ-NP led to significant enhancement of the tdTomato fluorescence intensity in comparison with the PBS-treated group (FIG. 16C). Consistent with the IVIS results, more tdTomato expression was found in the hypotonic NHEJ-NP-treated lung epithelium than the isotonic NHEJ-NP-treated one (FIG. 16D).

### EQUIVALENTS

While certain embodiments have been illustrated and described, a person with ordinary skill in the art, after reading the foregoing specification, can effect changes, substitutions of equivalents and other types of alterations to the methods and oligosaccharides of the present technology or derivatives, nutraceutical compositions, or pharmaceutical compositions thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, conditions, starting materials, reagents, compounds, or compositions, which can, of course, vary. All methods described herein can be performed in any suitable order unless otherwise indicated herein or otherwise clearly contradicted by context. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof. No language in the specification should be construed as indicating any non-claimed element as essential.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the phrase “consisting essentially of” will be understood to include those elements specifically recited and those additional elements that do not materially affect the basic and novel characteristics of the claimed technology. The phrase “consisting of” excludes any element not specified. The terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. More specifically, it will be understood that each use of terms such as “comprising,” “consisting essentially of,” or “consisting of”, discloses and provides written description and support for the use any of the other terms with the same or any other embodiment described herein.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the technology. This includes the generic description of the technology with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member, and each separate value is incorporated into the specification as if it were individually recited herein. Thus, for example, a group having 1-3 atoms refers to groups having 1, 2, or 3 atoms. Similarly, a group having 1-5 atoms refers to groups having 1, 2, 3, 4, or 5 atoms, and so forth.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims, along with the full scope of equivalents to which such claims are entitled.

1. A self-assembled nanoparticle comprising an amphiphilic copolymer, a ribonucleoprotein (RNP), and optionally ssODN, wherein:
the amphiphilic copolymer is a water-soluble block copolymer comprising a poly(C₂₋₃ alkylene glycol) block and an acrylic block comprising a poly(acrylate), poly(methacrylate) or poly(acrylate/methacrylate) block;
the acrylic block comprises ester side chains bearing substituted or unsubstituted alkylamine groups; and
the RNP, ssODN, and the acrylic block of the amphiphilic copolymer form a core of the self-assembled nanoparticle, and the poly(ethylene glycol) block of the amphiphilic copolymer forms the exterior of the self-assembled nanoparticle.

2. The self-assembled nanoparticle of claim 1 wherein the poly(C₂₋₃ alkylene glycol) block is a poly(ethylene glycol) block.

3. The self-assembled nanoparticle of claim 1 wherein the poly(C₂₋₃ alkylene glycol) block has a number average molecular weight of about 90 Da to about 20 kDa.

4. (canceled)

5. (canceled)

6. The self-assembled nanoparticle of claim 1 wherein the poly(C₂₋₃ alkylene glycol) block terminates with a moiety selected from hydroxy, an amino acid, a peptide, or substituted or unsubstituted alkyl, alkenyl, alkynyl, cycloalkyl, aryl, heteroalkyl, heteroaryl, or heterocyclyl group.

7. The self-assembled nanoparticle of claim 1 wherein the poly(C₂₋₃ alkylene glycol) block has the following structure of Formula I: 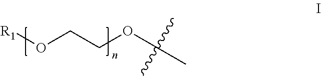
wherein
n is an integer from 2 to 500;
R¹ is H, substituted or unsubstituted alkyl, substituted or unsubstituted cycloalkyl, substituted or unsubstituted aryl, substituted or unsubstituted aralkyl, substituted or unsubstituted heteroalkyl, substituted or unsubstituted heterocyclyl, substituted or unsubstituted heterocyclylalkyl, or a moiety having a formula selected from the group consisting of 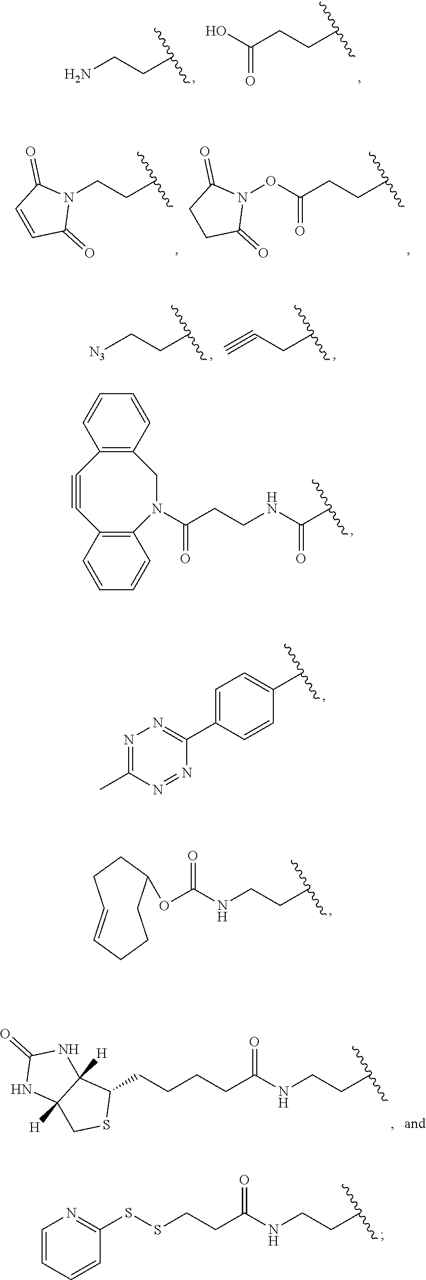
and
further wherein, for R¹ other than H, R¹ is or is optionally substituted with a fluorophore, a metal chelating group, a targeting ligand or a cell-penetrating peptide.

8. The self-assembled nanoparticle of claim 7, wherein R¹ is H, an unsubstituted C₁₋₄ alkyl group or a cell-penetrating peptide.

9. The self-assembled nanoparticle of claim 1, wherein the acrylic block consists of poly(methacrylate).

10. The self-assembled nanoparticle of claim 1, wherein the acrylic block has a number average molecular weight of about 500 Da to about 20 kDa.

11. (canceled)

12. The self-assembled nanoparticle of claim 1, wherein the acrylic block comprises a repeating subunit with the structure of Formula II: 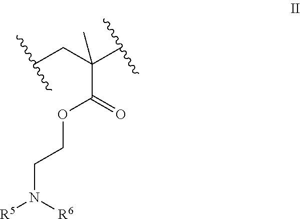
wherein:
R⁵ and R⁶ independently selected from H, substituted or unsubstituted alkyl, or substituted or unsubstituted cycloalkyl; or R⁵ and R⁶, together with the N atom to which they are attached, form a substituted or unsubstituted heterocyclyl.

13. The self-assembled nanoparticle of claim 1, wherein the acrylic block comprises two different repeating subunits having a structure of Formula II.

14. The self-assembled nanoparticle of claim 12, wherein the acrylic block comprises 5-150 of the repeating subunit.

15. The self-assembled nanoparticle of claim 1, wherein the acrylic block and the poly(ethylene glycol) block are joined by an ester, amide, ether, amine, thioether, or disulfide group.

16. The self-assembled nanoparticle of claim 1, wherein the amphiphilic polymer has the structure of Formula III: 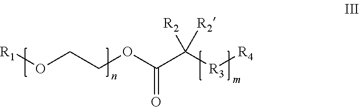
wherein
n is an integer from 2 to 500;
m is an integer from 2-150;
R¹ is H, substituted or unsubstituted alkyl, substituted or unsubstituted cycloalkyl, substituted or unsubstituted aryl, substituted or unsubstituted aralkyl, substituted or unsubstituted heteroalkyl, substituted or unsubstituted heterocyclyl, substituted or unsubstituted heterocyclylalkyl, or a moiety having a formula selected from the group consisting of 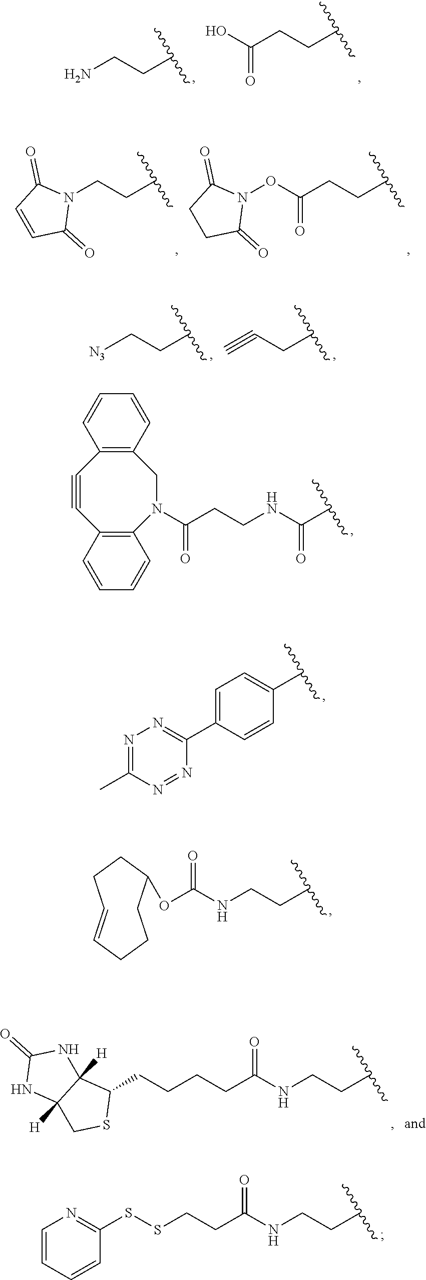
and
further wherein, for R¹ other than H, R¹ is or is optionally substituted with a fluorophore, a chemiluminescent entity or a phosphorescent entity, a metal chelating group, a targeting ligand, or a cell-penetrating peptide;
R² and R^{2′} are independently selected from the group consisting of H, substituted or unsubstituted alkyl, and substituted or unsubstituted cycloalkyl;
R³ has the structure: 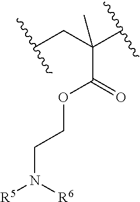
wherein:
R⁵ and R⁶ are independently selected from H, substituted or unsubstituted alkyl, or substituted or unsubstituted cycloalkyl, or R⁵ and R⁶, together with the N atom to which they are attached, form a substituted or unsubstituted heterocyclyl; and
R⁴ is H, OH, halo, or substituted or unsubstituted alkyl.

17. The self-assembled nanoparticle of claim 16, wherein R⁵ and R⁶ are independently selected from unsubstituted alkyl or R⁵ and R⁶, together with the N atom to which they are attached, form a C₄₋₈ heterocyclyl, optionally substituted with 1, 2, or 3 C₁₋₃ alkyl groups.

18. (canceled)

19. (canceled)

20. The self-assembled nanoparticle of claim 1, comprising ssODN.

21. The self-assembled nanoparticle of claim 20, wherein the RNP is selected from the group consisting of Cas9, Cas12, Cas 13, Cas14, and CasΦ.

22. The self-assembled nanoparticle of claim 20, wherein the ssODN is 20-200 nucleotides in length.

23. The self-assembled nanoparticle of claim 20, wherein the RNP is Cas9 and Cas9 comprises Cas9 nuclease and RNA selected from sgRNA or the combination of crRNA and tracrRNA.

24. The self-assembled nanoparticle of claim 23, wherein the RNA is sgRNA having a sequence selected from:
(SEQ ID NO: 31)
5′-NNNNNNNNNNNNNNNNNNNNGUUUUAGAGCUAGAAAUAGCAAGUUAA
AAUAAGGCUAGUCCGUUAUCAACUUGAAAAAGUGGCACCGAGUCGGUGCU
UUU-3′;
(SEQ ID NO: 32)
5′-NNNNNNNNNNNNNNNNNNNNGUUUAAGAGCUAUGCUGCGAAUACGAG
AUGCGGCCGCCGACCAGAAUCAUGCAAGUGCGUAAGAUAGUCGCGGGUCG
GCGGCUCGUAUUCGCAGCAUAGCAAGUUUAAAUAAGGCUAGUCCGUUAUC
AACUUGAAAAAGUGGCACCGAGUCGGUGCUUUU-3′;
(SEQ ID NO: 33)
5′-NNNNNNNNNNNNNNNNNNNNGUUUAAGAGCUAUGCUGGAAACAGCAU
AGCAAGUUUAAAAAGGCUAGUCCGUUAUCAACUUCGAAUACGAGAUGCGG
CCGCCGACCAGAAUCAUGCAAGUGCGUAAGAUAGUCGCGGGUCGGCGGCU
CGUAUUCGGAAAAAGUGGCACCGAGUCGGUGCUUUU-3′;
or
(SEQ ID NO: 34)
5′-NNNNNNNNNNNNNNNNNNNNGUUUAAGAGCUAUGCUGGAAACAGCAU
AGCAAGUUUAAAUAAGGCUAGUCCGUUAUCAACUUGAAAAAGUGGCACCG
AGUCGGUGCCGAAUACGAGAUGCGGCCGCCGACCAGAAUCAUGCAAGUGC
GUAAGAUAGUCGCGGGUCGGCGGCUCGUAUUCGUUUU-3′

25. The self-assembled nanoparticle of claim 23, wherein the RNA is the combination of crRNA and tracrRNA having sequences selected from:
SEQ ID NO: 35)
5′-NNNNNNNNNNNNNNNNNNNNGUUUUAGAGCUAUGCUGUUUUG-3′;
(SEQ ID NO: 36)
5′-UAGCAAGUUAAAAUAAGGCUAGUCCGUUAUCAACUUGAAAAAGUGGC
ACCGAGUCGGUGCUUUU-3′;
or
(SEQ ID NO: 37)
5′-GGAACCAUUCAAAACAGCAUAGCAAGUUAAAAUAAGGCUAGUCCGUU
AUCAACUUGAAAAAGUGCACCGAGUCGGUGCUUUU-3′.

26. The self-assembled nanoparticle of claim 20, wherein the RNP is Cas12 and the Cas12 comprises Cas12 nuclease and crRNA.

27. The self-assembled nanoparticle of claim 26, wherein the RNA is crRNA and/or tracrRNA having sequences selected from:
(SEQ ID NO: 38)
5′-UAAUUUCUACUCUUGUAGAUNNNNNNNNNNNNNNNNNNNNN-3′;
(SEQ ID NO: 39)
5′-GGUAAUUUCUACUAAGUGUAGAUNNNNNNNNNNNNNNNNNNNNNNN-
3′;
(SEQ ID NO: 40)
5′-GTCGGATCACTGAGCGAGCGATCTGAGAAGTGGCACNNNNNNNNNNN
NNNNNNNNN-3′;
or
(SEQ ID NO: 41)
5′-GTCTAAAGGACAGAATTTTCAACGGGTGTGCCAATGGCCACTTTCCA
GGTGGCAAAGCCCGTTGAACTTCTCAAAAAGAACGCTCAGTGTTCTGAC-
3′.

28. The self-assembled nanoparticle of claim 20, wherein the RNP is Cas13 and the Cas13 comprises Cas13 nuclease and crRNA.

29. The self-assembled nanoparticle of claim 28, wherein the RNA is crRNA having a sequence selected from:
(SEQ ID NO: 42)
5′-GAUUUAGACUACCCCAAAAACGAAGGGGACUAAAACNNNNNNNNNNN
NNNNNNNNNNNNNNNNN-3′;
(SEQ ID NO: 43)
5′-NNNNNNNNNNNNNNNNNNNNNNNNNNNNNNGUUGUGGAAGGUCCAGU
UUUGGGGGCUAUUACAACA-3′;
or
(SEQ ID NO: 44)
5′-AACCCCUACCAACUGGUCGGGGUUUGAAACNNNNNNNNNNNNNNNNN
NNNNNN-3′.

30. The self-assembled nanoparticle of claim 1, wherein the weight ratio of RNP to amphiphilic copolymer is 1:1 to 1:100.

31. A pharmaceutical composition comprising a self-assembled nanoparticle of claim 1 in a pharmaceutically acceptable aqueous carrier.

32.-37. (canceled)
